(19) United States
(12) Patent Application Publication
(10) Pub. No.: US 2011/0124063 A1
(43) Pub. Date: May 26, 2011

(54) Methods, Systems, and Compositions for Microbial Bio-Production of Biomolecules Using Syngas Components, or Sugars, as Feedstocks

(75) Inventor: Michael D. Lynch, Boulder, CO (US)

(73) Assignee: OPX Biotechnologies, Inc., Boulder, CO (US)

(21) Appl. No.: 12/950,863

(22) Filed: Nov. 19, 2010

Related U.S. Application Data
(60) Provisional application No. 61/263,249, filed on Nov. 20, 2009.

(51) Int. Cl. C12P 7/64; C12P 7/24; C12P 7/22; C12N 15/74; C12N 1/00; C12N 1/21

(52) U.S. Cl. 435/134; 435/147; 435/156; 435/471; 435/243; 435/252.3

(57) ABSTRACT

This invention relates to microorganism cells that are modified to increase conversion of carbon dioxide and/or carbon monoxide to a product, such as a fatty acid methyl ester, and to related methods and systems. A pathway from the Calvin Benson Cycle to the product is provided, which in various embodiments involves use of heterologous proteins that exhibit desired enzymatic conversions.

### RELATED APPLICATIONS

This application claims priority to U.S. Provisional Patent Application No. 61/263,249, filed Nov. 20, 2009, which is incorporated in its entirety herein.

### STATEMENT REGARDING FEDERALLY SPONSORED DEVELOPMENT

This invention was made with partial United States Government support under DE-AR0000088 awarded by the United States Department of Energy. The United States Government may have certain rights in this invention.

### REFERENCE TO A SEQUENCE LISTING

The instant application contains a Sequence Listing which has been submitted in ASCII format via EFS-Web and is hereby incorporated by reference in its entirety. Said ASCII copy, created on Nov. 19, 2010, is named 111910SequencesST25.txt and is 94.3 kB in size.

### FIELD OF THE INVENTION

The present invention relates to methods, systems and compositions, including genetically modified microorganisms, e.g., recombinant microorganisms, adapted to utilize one or more synthesis gas components in a microbial bio-production of one or more desired biomolecules of commercial interest.

### BACKGROUND

Economic, environmental and political impacts of and longer-term concerns with the current petroleum-based economy have driven the development and commercialization of processes that convert renewable feed stocks to both fuels and chemicals that can replace those derived from petroleum feed stocks. Two important goals of these developing processes include cost competitiveness with petroleum processes and reduced or net zero carbon dioxide or green house gas emissions. One approach to achieving these goals is the development of biorefining processes that utilize microorganisms to convert renewable feedstock sources such as cellulosic biomass or waste mass into products that are traditionally derived from petroleum or that can replace petroleum derived products. The list of petroleum-derived products of commercial value is exhaustive but includes molecules that fit into both the fuels and the chemicals markets, the latter including various industrial chemicals.

Due to recent competition between biorefining and food consumption for grains such as corn, and for sugar, it is clear that the path to sustainable non-petroleum-based fuel and chemical bio-production will require use of a broad range of alternative renewable feedstocks. One approach that may employ a wide range of alternative renewable feedstocks involves the thermo-conversion under oxygen-limited conditions of various carbonaceous feedstocks into synthesis gas.

Synthesis gas, which is also known as “syngas,” as used herein is a mixture of gases comprising carbon monoxide (CO), carbon dioxide (CO₂), and hydrogen (H₂) (collectively or individually, “syngas components”). Generally, syngas may be produced from any biomass material by gasification, steam reforming, partial oxidation, and similar processes that introduce oxygen at less than the stoichiometric ratio for combustion of the biomass. In some processes, part of the biomass is combusted, releasing CO₂ and heat which drives syngas formation from the biomass. Biomass such as lignocellulosic feedstocks, agricultural wastes, forest products, and grasses may be converted to syngas. In general, any carbonaceous feedstock can be utilized, including coal, petroleum, and natural gas, but renewable carbonaceous feedstocks such as biomass are considered particularly suitable. Gas mixtures derived from hydrogen and carbon dioxide produced from routes other than gasification could also be considered equivalents to syngas. For example, carbon dioxide waste streams may be mixed with hydrogen produced via any source for example electrolysis, steam methane reforming or any other.

Syngas is a platform intermediate in the chemical and biorefining industries and has a vast number of uses. Syngas can be converted into alkanes, olefins, oxygenates, and alcohols. These chemicals can be blended into, or used directly as, diesel fuel, gasoline, and other liquid fuels. Processes have been developed to convert syngas into chemicals such as methanol and acetic acid, and into liquid fuels using Fischer-Tropsch chemistry.

Components of syngas may be utilized in various ways, including as feedstock for biorefining processes. Production of syngas can be desirable within the context of bioconversion using microorganisms, because renewable biomass or waste feedstocks—which can be difficult to directly convert using microorganism—can first be converted into basic electron-rich reductant molecules H₂ and CO which can be consumed by suitable microorganisms.

A review of biological conversions of syngas is provided by Robert C. Brown in Chapter 11, pp. 227-252, of “Biorefinery Systems—An Overview,” in Biorefineries—Industrial Processes and Products, B. Kamm et al., Wiley-VCH (2006). This chapter is incorporated by reference herein for this background and descriptions of basic gasification reactions and certain metabolic pathways. According to this reference, anaerobic microorganisms have been favored for utilization of syngas conversions; this is stated to be because anaerobic microorganisms employ very energy-efficient metabolic pathways.

For example, U.S. Pat. No. 6,340,581, issued Jan. 22, 2002 to James L. Gaddy, discloses a method and apparatus for converting waste gases in a bioreactor to various products including organic acids and alcohols. Anaerobic bacteria are utilized in the bioreactor. Numerous specific microorganism isolates are disclosed, such as in the background section of US Patent Publication No. 2008/0057554, published Mar. 6, 2008 to R. L. Huhnke et al., and are stated to be used for production of biofuels and/or chemicals from syngas components (collectively, biomolecules of interest). An emphasis is placed on anaerobic microorganisms, particularly acetogens.

As to composition of the syngas components supplied to the microorganisms, the well-known water-gas shift reaction can be used to enrich for either the CO or the H₂ component of syngas. The water-gas shift reaction converts CO and H₂O into H₂ and CO₂. The reverse reaction also occurs, and the equilibrium of the water-gas shift reaction will generally govern the species distribution unless kinetic limitations are present. The water-gas shift can be performed on clean (i.e., purified) syngas, raw syngas directly from a gasification or partial-oxidation process, or any other source of syngas.

There is a clear need for alternative routes to create both fuels and products currently derived from petroleum. Fossil fuels account for 95% of the world energy usage and consumption of these fossil fuels has increased significantly over the last several decades. Consistent with this increase, carbon dioxide emissions have also been on a steady rise. These emissions are the primary reason for global climate change (//cnpublications.net/2009/04/24/biofuels-instead-of-gasoline/, Daniel Gorelick and guest blogger Chaitan Khosla and Harmit Vora, //www.springerlink.com/content/t78151r4811p6n74/, Jaime Klapp, Jorge L Cervantes-Cota, Luis C Longoria-Gandara and Ruslan Gabbasov). In addition to the environmental dilemma surrounding fossil fuels, there is also a federal interest in localizing energy production within the United States to reduce dependence on oil-producing foreign nations. Equally important, the localization of national energy production will lead to a growing American economy, thus creating more jobs. Microbial systems offer the potential for the biological production of numerous types of biofuels (//www.pubmedcentral.nih.gov/articlerender.fcgi?artid=2633538, Mattheos A. G. Koffas).

Biofuels, or synfuels, can be produced from a wide range of products, from coal, natural gas, or biomass feedstocks. Synfuels are created through chemical conversion into syncrude and/or synthetic liquid products (//www.eia.doe.gov/oiaf/aeo/otheranalysis/aeo_{—}2006analysispapers/figure_{—}19.html, //www.biodiesel.org/resources/faqs/, //repositories.cdlib.org/cgi/viewcontent.cgi?article=6799&context=lbnl/by Athanasios Lykidis). For example, biodiesel is a clean-burning alternative synfuel that can be produced from domestic renewable resources, such as switchgrass, rapeseed, or waste oils. “Biodiesel” is defined as mono-alkyl esters of long-chain fatty acids (//www.biodiesel.org/resources/faqs/). Common biodiesel constituents used today are fatty acid methyl esters (“FAMEs”). These fuels are derived from fatty acids obtained from triacylglycerols (TAGs), which are recovered from vegetable oils and animal fats (//repositories.cdlib.org/cgi/viewcontent.cgi?article=6799&context=lbnl/by Athanasios Lykidis). The advantage to biodiesel is that it is non-toxic, biodegradable, and has reduced sulfur emissions when compared to petroleum-based diesel fuel, thus having a lower output of greenhouse gasses when burned (//www.biodiesel.org/resources/faqs/).

Biodiesel constituents can in principle be derived from genetically engineered organisms, such as the bacteria E. coli (//cnpublications.net/2009/04/24/biofuels-instead-of-gasoline/, Daniel Gorelick and guest blogger Chaitan Khosla and Harmit Vora). Naturally occurring biosynthetic pathways of certain bacteria can be genetically altered to create new pathways which lead to an output of an energy-dense fuel product (//cnpublications.net/2009/04/24/biofuels-instead-of-gasoline/, Daniel Gorelick and guest blogger Chaitan Khosla and Harmit Vora). In addition, microbes can be tailored, or metabolically engineered, to utilize various carbon sources as feedstock for the production of oils, such as waste or agricultural byproducts (//repositories.cdlib.org/cgi/viewcontent.cgi?article=6799&context=lbnl/ by Athanasios Lykidis). Several forms of biodiesel produced by these organisms, including fatty acid methyl esters, are suitable for combustion directly in appropriate engines. These biofuels alleviate concerns revolving around food-crop usage for cellulosic ethanol, and concerns about global diversity (//www.thebioenergysite.com/articles/52/biofuel-and-global-biodiversity, Dennis Keeney and Claudia Nanninga).

It would be particularly beneficial for microorganisms to consume syngas components to produce biodiesel constituents to capture and contain the chemical energy released in the process (//www.biomassmagazine.com/articlejsp?article_id=1399). By employing syngas as the feedstock, typical byproducts such as glycol or glycerin can be avoided. Also, lower-cost feedstocks can ultimately be utilized, thereby enhancing overall economics and flexibility.

Notwithstanding the above-noted and other advances in the field, there remains a need to provide specific and, in some cases, coordinated improvements in microorganisms and biorefinery systems in which they would be utilized in order to achieve robust and cost-effective bio-production of biomolecules of interest from syngas components.

### SUMMARY

Some aspects of the invention relate to integrated thermochemical-biological processing facilities, in particular those that utilize genetically modified microorganisms. Other aspects relate to the methods utilized to construct such genetically modified microorganisms and their methods of use in the systems and facilities, including those focused on the use of syngas components to provide carbon and energy to genetically modified microorganisms. Other aspects teach the use of metabolic pathways described herein with one or more sugars as a carbon and energy source.

In some embodiments, the invention relates to a method of making a genetically modified microorganism comprising providing to a selected microorganism at least one genetic modification to introduce or increase one or more enzymatic activities selected from the group consisting of phosphoglucose isomerase, inositol-1-phosphate synthase, inositol monophosphatase, myo-inositol dehydrogenase, myo-inosose-2-dehydratase, inositol 2-dehydrogenase, deoxy-D-gluconate isomerase, 5-dehydro-2-deoxygluconokinase, deoxyphophogluconate aldolase, aldehyde dehydrogenase, malonyl-CoA synthetase, fatty acid synthase, and fatty acyl-CoA/ACP thioesterase. In various embodiments there may be two or more, three or more, four or more, five or more, and the like, up to all of the noted enzymatic activities, that are provided by the noted at least one genetic modification.

Further, in specific embodiments the genetic modifications, such as those used in the methods of the invention and in microorganism compositions of the invention, comprise adding one or more of the particular nucleic acid sequences provided in Table 1, incorporated herein, conservatively modified variants thereof, and/or functional variants thereof, so as to provide one or more desired enzymatic activity described in Table 1 and depicted as the numbered reactions in FIG. 1, also incorporated into this section. For example, a microorganism comprising the malonyl-CoA synthetase also may comprise an enzyme complex that is encoded by fabD, fabH, fabG, fabZ, fabI or fabK, fabF and fabB.

Also, the invention comprises a method of making a genetically modified microorganism comprising providing to a selected microorganism at least one genetic modification to introduce or increase one or more enzymatic activities provided by amino acid sequences having at least 50%, 60%, 70%, 80%, 85%, 90%, 92%, 95%, 96%, 97%, 98% or 99% sequence identity to one or more amino acid sequences selected from the group consisting of SEQ ID NO:002, SEQ ID NO:004, SEQ ID NO:006, SEQ ID NO:008, SEQ ID NO:010, SEQ ID NO:012, SEQ ID NO:014, SEQ ID NO:016, SEQ ID NO:018, SEQ ID NO:020, SEQ ID NO:022, SEQ ID NO:024, SEQ ID NO:026, and conservatively modified variants thereof.

Also, the invention comprises a method of making a genetically modified microorganism comprising providing to a selected microorganism at least one genetic modification comprising providing a polynucleotide comprising a nucleic acid sequence having at least 50%, 60%, 70%, 80%, 85%, 90%, 92%, 95%, 96%, 97%, 98% or 99% sequence identity to one or more nucleic acid sequences from the group consisting of SEQ ID NO:001, SEQ ID NO:003, SEQ ID NO:005, SEQ ID NO:007, SEQ ID NO:009, SEQ ID NO:011, SEQ ID NO:013, SEQ ID NO:015, SEQ ID NO:017, SEQ ID NO:019, SEQ ID NO:021, SEQ ID NO:023, SEQ ID NO:025, and conservatively modified variants thereof.

Also, for each of the respective nucleic acid and amino acid sequences provided herein, the invention comprises:

a. Any of the methods and compositions provided herein, having an amino acid sequence having at least 50%, 60%, 70%, 80%, 85%, 90%, 92%, 95%, 96%, 97%, 98% or 99% sequence identity to an amino acid sequence provided herein.

b. Any of the methods and compositions provided herein, having an amino acid sequence that is a functional variant of an amino acid sequence provided herein.

c. Any of the methods and compositions provided herein, having an amino acid sequence variant that stringently hybridizes to an amino acid sequence provided herein.

d. Any of the methods and compositions provided herein, having a polynucleotide (nucleic acid sequence) that encodes an amino acid sequence having at least 50%, 60%, 70%, 80%, 85%, 90%, 92%, 95%, 96%, 97%, 98% or 99% sequence identity to an amino acid sequence provided herein.

e. Any of the methods and compositions provided herein, having a polynucleotide (nucleic acid sequence) has at least 50%, 60%, 70%, 80%, 85%, 90%, 92%, 95%, 96%, 97%, 98% or 99% sequence identity to a polynucleotide sequence provided herein.

By amino acid and polynucleotide sequence (nucleic acid sequence) provided herein is meant one of the sequences of SEQ ID NO:001 to 032 and the sequences of the enzymes shown in FIGS. 2 and 3, discussed further herein.

The invention also comprises a method of making a genetically modified microorganism comprising providing to a selected microorganism at least one genetic modification to introduce or increase one or more enzymatic activities selected from the group consisting of S-adenosyl-homocysteine hydrolase, ribonuclease hydrolase-3, homocycsteine transmethylase, methionine adenosyltransferase, and O-methyltransferase. In various embodiments there may be two or more, three or more, four or more, or all five of the noted enzymatic activities, that are provided by the noted at least one genetic modification.

In particular methods and compositions, the S-adenosyl-homocysteine hydrolase is encoded by the Ahcy gene of R. norvegicus, the ribonuclease hydrolase-3 is encoded by the rihC gene of E. coli, the homocycsteine transmethylase is encoded by the metE gene of E. coli, and/or the methionine adenosyltransferase is encoded by the metK gene of E. coli, and the enzymatic activities are effective for achieving the conversions indicated in FIG. 3. Also, in particular methods and compositions, including genetically modified microorganisms, the O-methyltransferase comprises a Drosophila melanogaster juvenile hormone acid O-methyltransferase that has been modified to obtain a desired activity using a fatty acid as its substrate. Such O-methyltransferase may be a variant obtained by enzyme evolution to achieve the desired activity and specificity.

Additionally, other possible O-methyltransferase proteins may be employed, including an O-methyltransferase protein from the following list of microorganisms, or functional variants thereof and/or sequences in a selected microorganism, such as Oligotropha carboxidovorans or Cupriavidus necator, that are homologous to an O-methyltransferase protein as provided herein. In various embodiments, the method provides a higher yield of fatty acid methyl esters compared to an otherwise identical method with a microorganism lacking a heterologous nucleic acid molecule encoding an O-methyltransferase protein.

The scope of the invention includes microorganisms made by the methods described herein, and culture systems employing these microorganisms to produce FAMEs which may be used, for example, as a biodiesel fuel or in a blended diesel fuel. For example, the invention includes a culture system comprising (i) a population of genetically modified microorganisms as described herein and (ii) a media comprising nutrients for said population.

In various embodiments a microorganism is selected from chemolithotrophic bacteria, and more particularly may be Oligotropha carboxidovorans, Cupriavidus necator, or strain H16 of Cupriavidus necator.

More generally, the invention also includes a genetically modified microorganism comprising at least one genetic modification to introduce or increase one or more enzymatic activities selected from the group consisting of phosphoglucose isomerase, inositol-1-phosphate synthase, inositol monophosphatase, myo-inositol dehydrogenase, myo-inosose-2-dehydratase, inositol 2-dehydrogenase, deoxy-D-gluconate isomerase, 5-dehydro-2-deoxygluconokinase, deoxyphophogluconate aldolase, aldehyde dehydrogenase, malonyl-CoA synthetase, fatty acid synthase, fatty acyl-CoA/ACP thioesterase and carbon monoxide dehydrogenase. In various embodiments there may be two or more, three or more, four or more, five or more, and the like, up to all of the noted enzymatic activities, that are provided by the noted at least one genetic modification.

In particular embodiments the genetically modified microorganism may comprise a phosphoglucose isomerase encoded by the pgi gene of E. coli, a inositol-1-phosphate synthase encoded by the ino-1 gene of S. cerevisiae, an inositol monophosphatase encoded by the subB gene of E. coli, a myo-inositol dehydrogenase encoded by the iolG gene of B. subtilis, a myo-inosose-2-dehydratase encoded by the iolE gene of B. subtilis, an inositol 2-dehydrogenase encoded by the iolD gene of B. subtilis, a deoxy-D-gluconate isomerase encoded by the iolB gene of B. subtilis, a 5-dehydro-2-deoxygluconokinase encoded by the iolC gene of B. subtilis, a deoxyphophogluconate aldolase is encoded by the iolJ gene of B. subtilis, an aldehyde dehydrogenase is encoded by the aldA gene of E. coli, a matB gene of Rhizobium leguminosum, and/or a malonyl-CoA synthetase that comprises an enzyme complex encoded by fabD, fabH, fabG, fabZ, fabI or fabK, fabF and fabB.

In some embodiments, a genetically modified microorganism for the production of fatty acid methyl esters may comprise at least one heterologous nucleic acid molecule selected from the groups of nucleic acid molecules encoding a) O-methyltransferase; b) phosphoglucose isomerase, inositol-1-phosphate synthase, inositol monophosphatase, myo-inositol dehydrogenase, myo-inosose-2-dehydratase, inositol 2-dehydrogenase, deoxy-D-gluconate isomerase, 5-dehydro-2-deoxygluconokinase, deoxyphophogluconate aldolase, aldehyde dehydrogenase, malonyl-CoA synthetase, fatty acid synthase enzymes, and fatty acyl-CoA/ACP thioesterase; and/or c) S-adenosyl-homocysteine hydrolase, ribonuclease hydrolase-3, homocycsteine transmethylase, and methionine adenosyltransferase. In addition, the genetically modified microorganism may include a number of genetic modifications such as at least two, at least three, at least four, at least five, at least six, at least seven, at least eight, at least nine, at least ten, at least eleven, and at least twelve enzymatic activities.

In some embodiments, the genetically modified microorganism is selected from the group consisting of chemolithotrophic bacteria. In some embodiments, the genetically modified microorganism is selected from the group consisting Oligotropha carboxidovorans, Cupriavidus necator, and strain H16 of Cupriavidus necator.

A genetically modified microorganism of the present invention may comprise at least one genetic modification to introduce or increase one or more enzymatic activities provided by amino acid sequences having at least 50%, 60%, 70%, 80%, 85%, 90%, 92%, 95%, 96%, 97%, 98% or 99% sequence identity to one or more amino acid sequences selected from the group consisting of SEQ ID NO:002, SEQ ID NO:004, SEQ ID NO:006, SEQ ID NO:008, SEQ ID NO:010, SEQ ID NO:012, SEQ ID NO:014, SEQ ID NO:016, SEQ ID NO:018, SEQ ID NO:020, SEQ ID NO:022, SEQ ID NO:024, SEQ ID NO:026, and conservatively modified variants thereof. In various embodiments there may be two or more, three or more, four or more, five or more, and the like, up to all of the noted enzymatic activities, that are provided by the noted at least one genetic modification.

Also, a genetically modified microorganism of the invention may comprise at least one genetic modification provided by a polynucleotide comprising a nucleic acid sequence having at least 50%, 60%, 70%, 80%, 85%, 90%, 92%, 95%, 96%, 97%, 98% or 99% sequence identity to one or more nucleic acid sequences from the group consisting of SEQ ID NO:001, SEQ ID NO:003, SEQ ID NO:005, SEQ ID NO:007, SEQ ID NO:009, SEQ ID NO:011, SEQ ID NO:013, SEQ ID NO:015, SEQ ID NO:017, SEQ ID NO:019, SEQ ID NO:021, SEQ ID NO:023, SEQ ID NO:025, and conservatively modified variants thereof. In various embodiments there may be two or more, three or more, four or more, five or more, and the like, up to all of the noted enzymatic activities, that are provided by the noted at least one genetic modification.

The invention also comprises a method of making a genetically modified microorganism comprising providing to a selected microorganism at least one genetic modification to decrease one or more enzymatic activities selected from the group consisting of fatty acyl-coA synthetase, fatty acyl-coA dehydrogenase, polyhydroxybutyrate polymerase, acetoacetyl-coA reductase, acetyl-coA acetyltransferase, serine deaminase or methionine gamma lyase. In various embodiments there may be two or more, three or more, four or more, or all five of the noted enzymatic activities, that are provided by the noted at least one genetic modification. A genetically modified microorganism, including any of the above-described genetically modified microorganisms, also may comprise at least one genetic modification to introduce or increase one or more enzymatic activities selected from the group consisting of S-adenosyl-homocysteine hydrolase, ribonuclease hydrolase-3, homocysteine transmethylase, methionine adenosyltransferase, and O-methyltransferase. In various embodiments there may be two or more, three or more, four or more, or all five of the noted enzymatic activities, that are provided by the noted at least one genetic modification.

Any such genetically modified microorganism may provide for the conversion of S-adenosylmethionine to S-adenosyl-homocysteine that releases a methyl group for combining with the fatty acid to generate said fatty acid methyl ester.

In some embodiments, the method is a method for producing malonate semialdehyde comprising: a) combining hydrogen, a carbon source selected from carbon monoxide and carbon dioxide, and a culture of microorganism cells, wherein said microorganism cells comprise at least one genetic modification to introduce or increase one or more enzymatic activities selected from the group consisting of phosphoglucose isomerase, inositol-1-phosphate synthase, inositol monophosphatase, myo-inositol dehydrogenase, myo-inosose-2-dehydratase, inositol 2-dehydrogenase, deoxy-D-gluconate isomerase, 5-dehydro-2-deoxygluconokinase, and deoxyphophogluconate aldolase; and b) maintaining the combined hydrogen, carbon source, and microorganism cells for a suitable time and under conditions sufficient to convert the carbon source to malonate semialdehyde. The microorganism may be capable of converting the carbon source to fructose-6-phosphate. The malonate semialdehyde so produced may be further processed to yield an organic compound such as fatty acid methyl ester. The microorganisms producing the malonate semialdehyde may be modified to comprise a heterologous nucleic acid molecule encoding an O-methyltransferase protein; aldehyde dehydrogenase, malonyl-CoA synthetase, fatty acid synthetase complex, fatty acyl-CoA/ACP thioesterase proteins, aldehyde dehydrogenase, malonyl-CoA synthetase, fatty acid synthase complex, and/or fatty acyl-CoA/ACP thioesterase proteins.

In some embodiments, the method is a method for producing myo-inositol comprising: a) combining hydrogen, a carbon source selected from carbon monoxide and carbon dioxide, and a culture of microorganism cells, wherein said microorganism cells comprise at least one genetic modification to introduce or increase one or more enzymatic activities selected from the group consisting of phosphoglucose isomerase, inositol-1-phosphate synthase, inositol monophosphatase, myo-inositol dehydrogenase, myo-inosose-2-dehydratase, inositol 2-dehydrogenase, deoxy-D-gluconate isomerase, 5-dehydro-2-deoxygluconokinase, and deoxyphophogluconate aldolase; and b) maintaining the combined hydrogen, carbon source, and microorganism cells for a suitable time and under conditions sufficient to convert the carbon source to myo-inositol. The microorganism may be capable of converting the carbon source to fructose-6-phosphate. The myo-inositol so produced may be further processed to yield an organic compound such as fatty acid methyl ester. The microorganisms producing the myo-inositol may be modified to comprise a heterologous nucleic acid molecule encoding an O-methyltransferase protein; aldehyde dehydrogenase, malonyl-CoA synthetase, fatty acid synthetase complex, fatty acyl-CoA/ACP thioesterase proteins, aldehyde dehydrogenase, malonyl-CoA synthetase, fatty acid synthase complex, and/or fatty acyl-CoA/ACP thioesterase proteins. The invention also includes a method of converting one or more syngas components, such as carbon dioxide or carbon monoxide and hydrogen, into a fatty acid, said method comprising feeding one or more syngas components to a solution comprising a genetically modified microorganism of the invention, as described herein, under suitable fermentation conditions which may be aerobic or anaerobic. In various embodiments of such method the volumetric productivity for fatty acid methyl esters is at least 0.5 g/L-hr, 1 g/L-hr, or at least 2 g/L-hr. In other various embodiments of such method the specific productivity for fatty acid methyl esters is at least 0.005 g/gDCW-hr, 0.05 g/gDCW-hr, 1 g/gDCW-hr, or at least 2 g/gDCW-hr. In some embodiments other feedstocks may be provided, including one or more sugars. For example, the carbon source may have an amount of glucose, sucrose, fructose, dextrose, lactose, glycerol, and/or combinations thereof that is selected from the group consisting of less than about 50%, less than about 40%, less than about 30%, less than about 20%, less than about 10%, less than about 5%, and less than about 1% by weight.

In some embodiments, the invention is directed to a method for producing fatty acid methyl esters comprising: combining hydrogen, a carbon source selected from carbon monoxide and carbon dioxide, and a culture of microorganism cells, wherein said microorganism cells comprise a heterologous nucleic acid molecule encoding an O-methyltransferase protein; and maintaining the combined hydrogen, carbon source, and microorganism cells for a suitable time and under conditions sufficient to convert the carbon source to fatty acid methyl esters. The carbon source may have a ratio of carbon-14 to carbon-12 of about 1.0×10-14 or greater. In various embodiments, the carbon source has a percentage of petroleum origin selected from less than about 50%, less than about 40%, less than about 30%, less than about 20%, less than about 10%, less than about 5%, less than about 1%, or essentially free of petroleum origin.

In various embodiments, the method of producing fatty acid methyl esters does not require the presence of a chemical catalyst for the conversion of the carbon source to fatty acid methyl esters. The fatty acid methyl esters may include a mixture of fatty acid moieties, or may be homogeneous with respect to the fatty acid moieties.

Further, the invention also includes a method of converting one or more sugars to FAMEs using one or more pathways provided herein, such as provided in FIGS. 1, 2 and 3. In various embodiments of such method the volumetric productivity for fatty acid methyl esters is at least 0.5 g/L-hr 1 g/L-hr, or at least 2 g/L-hr.

In various embodiments the efficiency of the conversions of carbon monoxide and/or carbon dioxide to any one of the organic compounds described herein is at least 2 percent, at least 10 percent, at least 50 percent, at least 60 percent, at least 70 percent, at least 80 percent, and at least 90 percent. In various embodiments, the percentage of carbon source converted to fatty acid methyl esters is selected from greater than 25%, greater than 35%, greater than 45%, greater than 55%, greater than 65%, greater than 75%, greater than 85%, and greater than 95%. Fatty acid methyl esters (FAMEs) produced according to the invention may be further processed to conform to one or more ASTM diesel fuel oil blend standards.

The invention also provides a culture system comprising (a) a population of a genetically modified microorganism as described herein and (b) a media comprising nutrients for said population.

The invention also provides a method of making a fatty acid molecule comprising: a) providing one or more genetic modifications to a selected microorganism host cell to obtain all enzymatic conversion steps depicted in FIG. 1 in said host cell; b) providing a supply of carbon dioxide and/or carbon monoxide, and hydrogen to said host cell; and c) culturing the cell under conditions suitable for production of fatty acids from the carbon dioxide and hydrogen. The invention also provides a method of making a fatty acid methyl ester molecule comprising: a) providing one or more genetic modifications to a selected microorganism host cell to obtain all enzymatic conversion steps depicted in FIG. 1 in said host cell; b) providing one or more genetic modifications to a selected microorganism host cell to obtain all enzymatic conversion steps depicted in FIG. 1 in said host cell; c) providing a supply of carbon dioxide, and/or carbon monoxide, and hydrogen to said host cell; and d) culturing the cell under conditions suitable for production of fatty acid methyl esters from the carbon dioxide and hydrogen.

### BRIEF DESCRIPTION OF THE DRAWINGS

FIG. 1 is an exemplary genetically modified pathway for producing fatty acids from syngas components, according to some variations of the invention.

FIG. 2 provides specific candidate reference sequences for Cupriavidus necator and Oligotropha carboxidovorans regarding the enzymes that catalyze the numbered steps in FIG. 1.

FIG. 3 is an exemplary genetically modified pathway for producing fatty acid methyl esters from fatty acids, according to some variations of the invention. The indicated genes are exemplary and not meant to be limiting.

FIG. 4 depicts reactions of a fatty acid synthase complex of E. coli and also indicates the reaction of a thioesterase.

FIGS. 5A and B depicts the reactions of a native versus an evolved form of an O-methyltransferase.

FIG. 6 provides additional specific candidate reference sequences for Oligotropha carboxidovorans regarding the enzymes that catalyze the numbered steps in FIG. 1.

FIG. 7 provides an example of construction of C. necator strains for evaluation.

FIG. 8 is Table 1 summarizing information regarding the enzymes that catalyze the numbered steps in FIG. 1.

FIG. 9 is Table 2 providing a summary of similarities among amino acids, upon which conservative and less conservative substitutions may be based.

Tables provided also comprise part of the invention.

### DETAILED DESCRIPTION OF THE INVENTION AND EMBODIMENTS THEREOF

Unless otherwise indicated, all numbers expressing reaction conditions, stoichiometries, sequence similarities, and so forth used in the specification and claims are to be understood as being modified in all instances by the term “about.” Accordingly, unless indicated to the contrary, the numerical parameters set forth in the following specification and attached claims are approximations that may vary depending at least upon the specific analytical technique. Any numerical value inherently contains certain errors necessarily resulting from the standard deviation found in its respective testing measurements.

As used in this specification and the appended claims, the singular forms “a,” “an,” and “the” include plural referents unless the context clearly indicates otherwise. Thus, for example, reference to an “expression vector” includes a single expression vector as well as a plurality of expression vectors, either the same (e.g., the same operon) or different; reference to “microorganism” includes a single microorganism as well as a plurality of microorganisms; and the like.

Unless defined otherwise, all technical and scientific terms used herein have the same meaning as is commonly understood by one of ordinary skill in the art to which this invention belongs. If a definition set forth in this section is contrary to or otherwise inconsistent with a definition set forth in patents, published patent applications, and other publications that are herein incorporated by reference, the definition set forth in this specification prevails over the definition that is incorporated herein by reference.

Certain particular embodiments of the present invention will be described in more detail, including reference to the accompanying figures and tables. The figures are understood to provide representative illustration of the invention and are not limiting in their content or scale. It will be understood by one of ordinary skill in the art that the scope of the invention extends beyond the specific embodiments depicted. This invention also incorporates routine experimentation and optimization of the methods, apparatus, and systems described herein.

There are several groups of bacteria able to utilize the primary components of synthesis gas, mainly H₂ (hydrogen) and CO (carbon monoxide), as sole sources of carbon and energy. One such group is known as chemolithotrophic bacteria, which are able to aerobically utilize carbon dioxide as a carbon source while oxidizing other inorganic sources of energy. This diverse group of bacteria includes ammonia oxidizers, nitrite oxidizers, sulfur oxidizers, iron oxidizers, hydrogen oxidizers, and carbon monoxide oxidizers. Two important aerobic chemolithotrophs include Cupriavidus necator (formerly known as Ralstonia eutropha) and Oligotropha carboxidovorans (formerly known as Pseudomonas carboxidovorans). Cupriavidus necator is able to oxidize hydrogen, while Oligotropha carboxidovorans is able to oxidize carbon monoxide, both in an aerobic environment. Another group of syngas utilizers is anaerobic bacteria or archea that are able to fix carbon monoxide through the reductive acetyl-coA pathway.

In some variations, this invention describes and provides metabolic pathways for the production of biodiesel or FAMEs and related products in aerobic chemolithotropes, such as Cupriavidus necator. This group of bacteria can fix carbon dioxide through the Calvin Benson Cycle (CBC), which is the same carbon-fixation cycle used by photosynthetic organisms. In Cupriavidus, this central pathway uses electrons and energy obtained from the oxidation of hydrogen which generates the NADPH and ATP needed for biosynthesis. C. necator is able to obtain reductants and energy needs from hydrogen by using two oxygen-tolerant hydrogenases: a soluble hydrogenase and a membrane-bound hydrogenase.

Cupriavidus necator has been characterized to have very high growth rates when grown chemolithotrophically on mixtures of hydrogen and carbon dioxide gases in an aerobic environment (Repaske and Mayer R, “Dense autotrophic cultures of Alcaligenes eutrophus AEM, 32(4), 592-597, 1976). In this species, it is believed (without the present invention being limited to any particular theory) that carbon fixation occurs exclusively through the Calvin Benson Cycle and all cell mass is generated from flux through this pathway. Numerous studies in the literature have shown that productivity through the Calvin Benson Cycle can achieve at least 20 g/L of biomass in 18 hours, or a specific volumetric productivity of approximately 1.34 g/L/hr, under non-optimized conditions and in standard stirred tanks.

The Calvin Benson Cycle is utilized by several chemolithotropic microbes including Oligotropha carboxidovorans and Cupriavidus necator, which can obtain electrons directly from syngas constituents. The megaplasmid pHCG3 of O. carboxidovorans is reported to comprise genes for utilization of CO, CO₂, and/or H₂. Strain H16 of Cupriavidus necator, previously called Ralstonia eutropha, is reported to comprise nucleic acid sequences encoding two hydrogenases and the enzymes of the Calvin Benson Cycle on the megaplasmid pHG1. C. necator has been used commercially to produce polyhydroxyalkanoates (a natural product from this organism) or natural polyester plastics (see, for example, U.S. Pat. Nos. 6,316,262, 6,689,589, 7,081,357, and 7,229,804). The genomic sequence of Cupriavidus necator is known and the genomic DNA sequence of Oligotropha carboxidovorans has recently been published (Genome Announcement Genome Sequence of Chemolithotrophic Bacterium Oligotropha carboxidovorans OM5^{T}”, Debarati Paul et al., J. of Bacteria 2008:190(5):5531-5532).

The reductive acetyl-CoA cycle is used by many anaerobic microorganisms including methanogens and acetogens. In this cycle, electrons and carbon from CO are used to produce larger molecules. Organisms utilizing this pathway tend to be strict anaerobes and many of the enzymes involved in the cycle itself are very sensitive to the presence of oxygen which inactivates them. This cycle produces acetyl-coA that may then be biologically converted to other products of interest.

The reductive tricarboxylic acid cycle (“TCA”) cycle is used primarily by anaerobic photosynthetic microorganisms. In this cycle CO₂ is fixed into acetyl-CoA by a reverse of the tricarboxylic acid cycle. Many organisms using this fixation cycle are strictly anaerobic and the enzymes that are involved in the cycle are not oxygen tolerant. However, several oxygen-tolerant enzymes involved in this cycle have been characterized.

The 3-hydroxypropionic acid cycle is used primarily by photosynthetic microorganisms. In this cycle CO₂ is fixed into glyoxylate through the intermediate 3-hydroxypropionate. Many organisms using this fixation cycle are thermophilic and the enzymes that are involved in the cycle operate optimally at elevated temperatures above 50° C.

Thus, several CO₂ fixation pathways such as the above have been characterized. These metabolic pathways use NADH or NADPH as electron carriers for the reduction and fixation of CO₂. In many aerobic photosynthetic organisms such as plants, these carriers are reduced with electrons from water obtained by light-driven reactions. CO and H₂ can be used to reduce these carriers as well. In particular, hydrogenases and CO dehydrogenases are enzymes that can catalyze the transfer of electrons from H₂ and CO, respectively, to NAD⁺ and NADP⁺. Oxygen-tolerant hydrogenases and CO dehydrogenases have been characterized that can carry out these reactions in the presence of oxygen (Bleijlevens et al., “The Auxiliary Protein HypX Provides Oxygen Tolerance to the Soluble [NiFe]-Hydrogenase of Ralstonia eutropha H16 by Way of a Cyanide Ligand to Nickel,” J. Biol. Chem. (2004)279:45, 46686-46691).

Many known bioprocesses utilizing syngas components require anaerobic environments due to the sensitivity of the microorganisms and their enzymes to oxygen. This requirement presents several hurdles and limitations in the bioconversion process. Fixation of CO₂ in these organisms is intimately tied to oxidation of CO or H₂ or to anaerobic cellular respiration.

In an aerobic environment, the reductants NADH and FADH₂ can be used by microorganisms to reduce oxygen to water via aerobic respiration. This allows for the production of energy and ATP via aerobic respiration, independently from CO₂ fixation. An aerobic bioconversion can allow for the microorganism to generate energy for processes other than cellular respiration, such as growth or tolerance to product or feedstock. In addition, the independent production of ATP from CO₂ fixation can allow for the production of higher-energy products from syngas components. In particular, metabolic pathways that utilize ATP to drive the formation of higher-energy products can be achieved.

The variations provided herein related to aerobic processes are consonant with increased microorganism productivities, flexibility of products, product and feedstock tolerance and aerobic respiration, all of which are important issues to be addressed for the successful commercialization of new biofuels and/or bioprocessed chemicals produced from syngas.

An important step in fatty acid synthesis is the biosynthesis of the intermediate malonyl-coA. The production of malonyl-coA is the committed step of fatty acid biosynthesis and is tightly regulated. Malonyl-coA is almost exclusively produced biologically by the action of acetyl-coA carboxylase enzymes. These enzymes tend to be complex multi-subunit enzymes that are regulated both at the transcriptional and protein or enzyme level. The regulation of prototypical acetyl-coA carboxylase from E. coli has been well-studied and includes regulation of this enzyme by the intermediates and products of fatty acid synthesis, such as fatty acyl-ACPs.

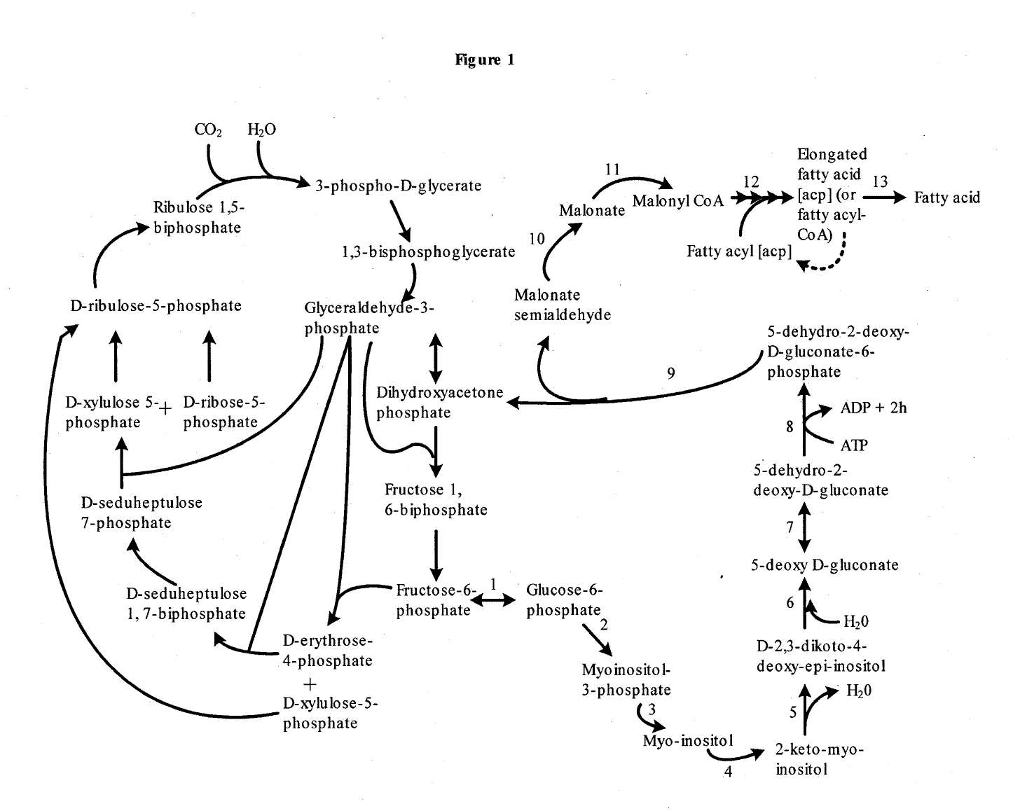

FIG. 1 depicts a metabolic pathway for producing free fatty acids from syngas through malonyl-CoA which may be provided or completed in a microorganism by genetic modification. The malonyl-CoA is generated from intermediates of the Calvin Benson Cycle, which is depicted on the left side of FIG. 1. It is noted that the FIG. 1 is a summary of the biological reactions that occur. That is, single arrows do not necessarily mean a single enzymatic step, and all of the reactants and products of each step are not necessarily shown. The numbers near arrows in FIG. 1 refer to step numbers as further described in Table 1 herein.

In microorganism genetically modified host cells, and methods and systems comprising such cells, the metabolic reactions depicted in FIG. 1 transpire to yield fatty acid molecules via malonyl-CoA, which may be derived from carbon dioxide and hydrogen (which in various embodiments are syngas constituents). The latter two compounds enter the Calvin Benson Cycle as shown in FIG. 1, and a later product of the Calvin Benson Cycle, fructose-6-phosphate, is converted to glucose-6-phosphate by a phosphoglucose isomerase. This reaction step begins a side route from the Calvin Benson Cycle that results in the production of dihydroxyacetone phosphate, which may return to and replenish the Calvin Benson Cycle, and malonate semialdehyde, which is converted sequentially to malonate, malonyl-CoA, a fatty acyl-CoA, and then a fatty acid. Thus, during this series of enzymatic reactions, malonate is produced, via several steps, from degradation of myo-inositol which is generated from the glucose-6-phosphate. The net result of this pathway is the generation of malonyl-coA for fatty acid synthesis and dihydroxyacetone phosphate which can be returned to the Calvin Benson Cycle. Three ATP molecules are consumed, providing a thermodynamic driving force for the pathway. This energetically favorable pathway bypasses the normal regulation of malonyl-coA synthesis. Whether the feedstock is syngas or sugars, there can be several entry points for feed components within the metabolic pathway of FIG. 1.

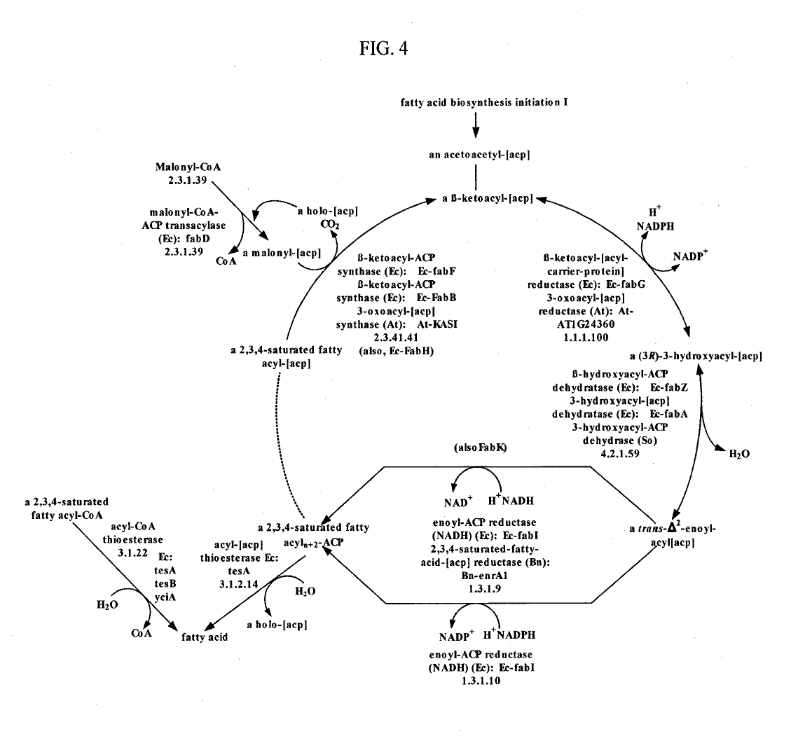

Table 1 summarizes information regarding the enzymes that catalyze the numbered steps in FIG. 1, including enzyme names, representative genes of species that encode for the specific enzymes, and relevant SEQ ID NOs. for the representative genes and their amino acid products. In various embodiments, this invention provides a method of making a genetically modified microorganism comprising providing to a selected microorganism at least one genetic modification to introduce or increase one or more enzymatic activities selected from the group consisting of phosphoglucose isomerase, inositol-1-phosphate synthase, inositol monophosphatase, myo-inositol dehydrogenase, myo-inosose-2-dehydratase, inositol 2-dehydrogenase, deoxy-D-gluconate isomerase, 5-dehydro-2-deoxygluconokinase, deoxyphosphogluconate aldolase, aldehyde dehydrogenase, malonyl-CoA synthetase, fatty acid synthase (which may be any suitable complex), and a thioesterase such as a fatty acyl-CoA/ACP thioesterase. In various distinct embodiments, these may be provided in any combination, including any combination of the proteins of Table 1 and FIG. 1, steps 1-13, and further of FIG. 4.

As used herein, by the terms “fatty acid synthase,” fatty acid synthase system, and the like, are meant the set of proteins in a microorganism cell that perform the following conversion: condensing a malonyl-CoA or a malonyl-[ACP] with a fatty acyl-CoA or a fatty acyl-[ACP]; reducing the elongated B-ketoacyl[ACP] or B-ketoacyl-CoA; dehydrating the so-formed hydroxyacyl molecule to an enoyl-acyl[ACP] or enoyl-acyl-CoA, and then reducing this to a so-elongated fatty acyl-[ACP] or fatty acyl-CoA. This can then go through further elongations until a sufficient length for further reactions described herein. This reaction generally starts with a C4 or greater alkyl molecule.

Thus, by providing polypeptides (particularly proteins) that catalyze enzymatic conversion steps of the myo-inositol pathway and enzymatic conversions of other steps, as provided in FIG. 1 and Table 1, in a microorganism host cell that comprises Calvin Benson Cycle capability, carbon dioxide and hydrogen, such as from a syngas process, are converted into fatty acid molecules. These fatty acid molecules may then be utilized in the production route described below toward production of bio-diesel FAMEs and other products, as described, for example, below.

Viewed another way, malonyl-CoA generated from the Calvin Benson Cycle can serve as an unregulated source of the malonyl-CoA precursor for fatty acid synthesis. Free fatty acids can be produced from fatty acyl-ACPs produced by native fatty acid synthase complexes via the action of numerous thioesterases including that encoded by the E. coli tesA gene. Additionally, it is noted that alternative thioesterases to the specific fatty acyl-CoA/ACP thioesterase recited above may be used and provided into a microorganism cell (as a heterologous nucleic acid/protein) in embodiments of the invention. These may lead to increased production of fatty acids, and/or to various derivatives of fatty acids. In these regards, U.S. Patent Application No. 2010/0154293, published Jun. 24, 2010, and incorporated by reference for its teachings of use of various thioesterases and their uses, including how to make fatty acids and fatty acid derivative products, and those products.

Another alternative pathway to fatty acid molecules is to proceed through all or part of the glycolysis pathway. For example, referring to FIG. 1, a fatty acid molecule may be derived from 1,3-diphosphoglycerate (also known as 1,3-bi-phosphoglycerate) via a portion of a glycolytic pathway. In such example, 1,3-diphosphoglycerate is converted enzymatically to 3-phospho-D-glycerate by a phosphoglycerate kinase (EC 2.7.2.3), which is converted enzymatically to 2-phospho-D-glycerate by a phosphoglycerate mutase (EC 5.4.2.1), which is converted enzymatically to phosphoenolpyruvate (PEP) by an enolase (EC 4.2.1.11). PEP is converted enzymatically to pyruvate such as by a pyruvate kinase (EC 2.7.1.40). Pyruvate is converted enzymatically to acetyl-CoA such as by a pyruvate dehydrogenase, typically in a pyruvate dehydrogenase multienzyme complex (e.g., ECs 1.2.4.1, 2.3.1.12, and 1.8.1.4). In various embodiments any combination of such enzymes are provided to a genetically modified microorganism that may also comprise other modifications as described herein, so as to produce a fatty acid molecule. In other embodiments wherein a carbon source in addition to or other than carbon dioxide or carbon monoxide is provided to a microorganism or culture thereof, the entire glycolysis pathway may be utilized to generate additional acetyl-CoA molecules that are then converted to malonyl-CoA molecules, which are then converted to fatty acid molecules (and other products) as described elsewhere herein.

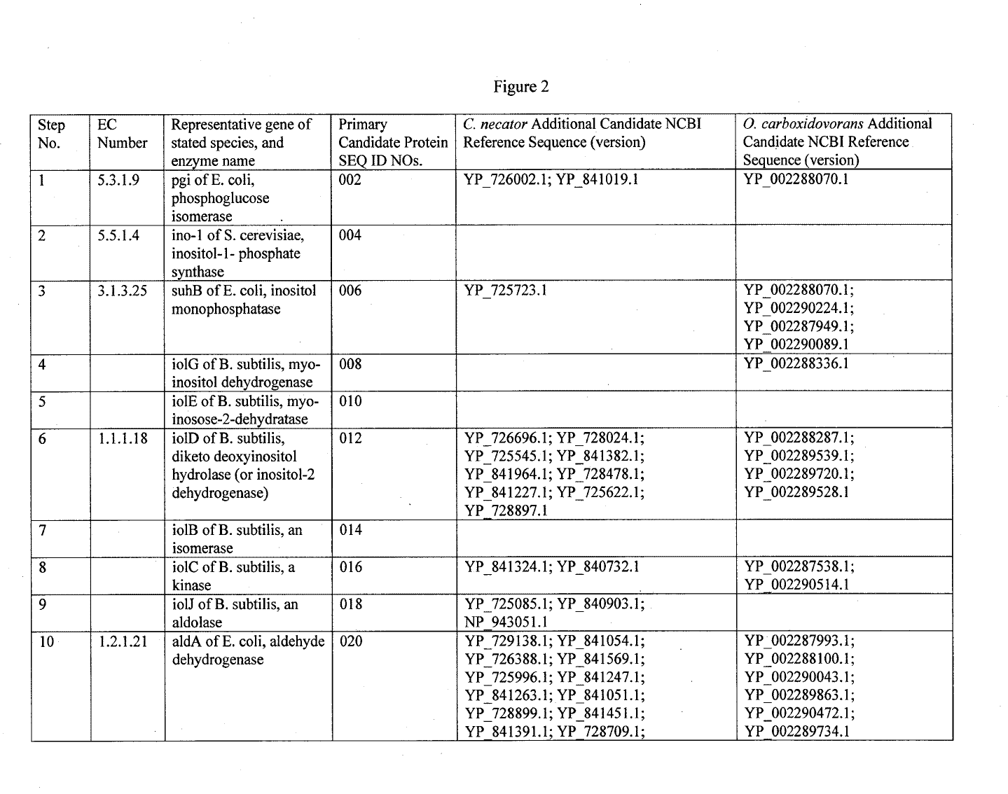

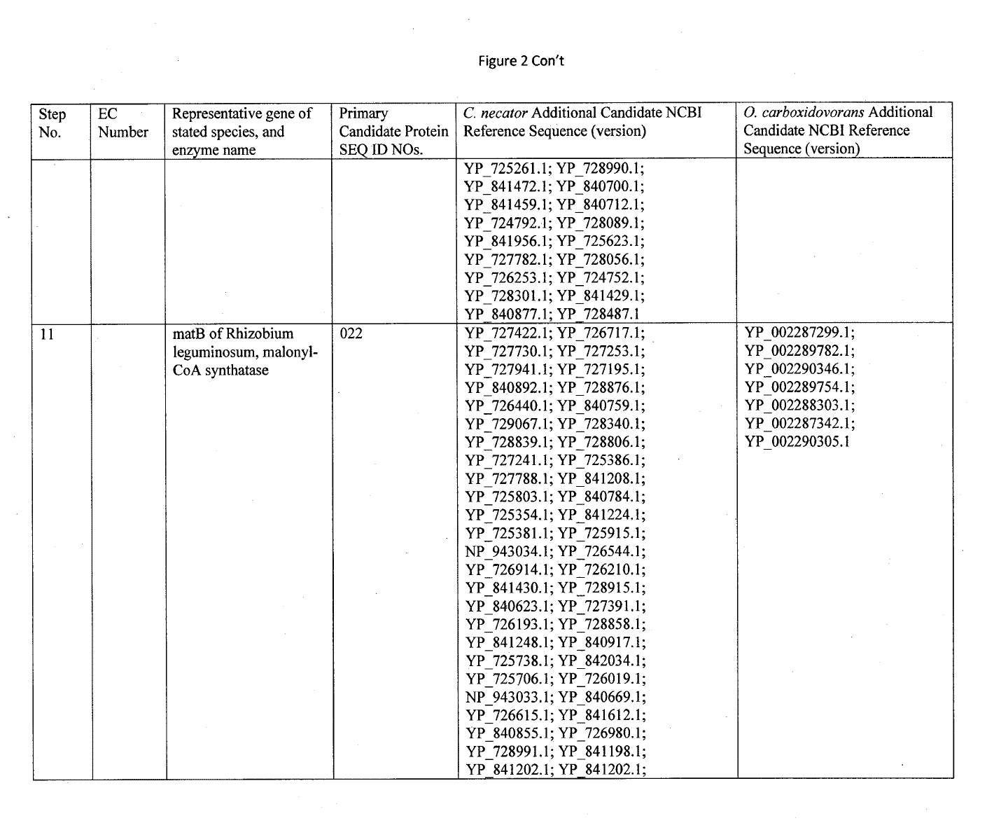

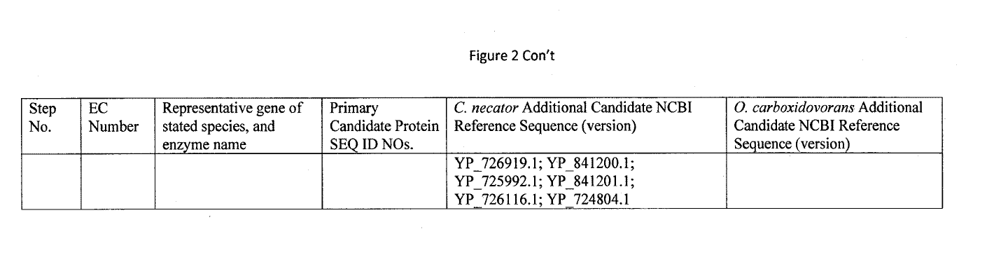

Expression or increased expression of the glycolysis metabolic pathway to increase production of fatty acid molecules in a modified microorganism of the present invention involves introducing one or more, or all, of the proteins, and their corresponding enzymatic activities of Table 1, and/or FIG. 2, which also provides specific candidate reference sequences for Cupriavidus necator and Oligotropha carboxidovorans.

As noted above, the reductive acetyl-coA pathway yields acetyl-coA. Accordingly, in some embodiments this pathway also may be used in a microorganism or culture thereof to increase production of fatty acids and related products. U.S. Pat. No. 7,803,589, granted Sep. 28, 2010, is incorporated by reference herein specifically for its teachings of microorganisms that comprise one or more exogenous (heterologous) proteins that confer to such microorganisms functionality of this pathway. These teachings may be applied and adapted to particular microorganisms which may comprise embodiments of the present invention. In a particular microorganism carbon dioxide (and/or carbon monoxide) and hydrogen may be converted to a fatty acid using one or more of the carbon fixation pathways described herein, and optionally also including the approach described herein to form a fatty acid ester, such as a fatty acid methyl ester.

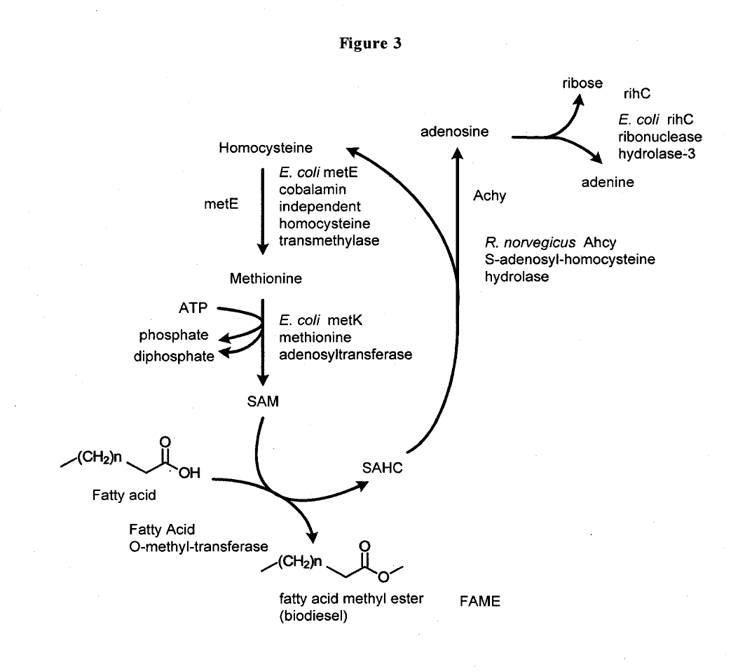

Accordingly, in some variations, whether a fatty acid molecule is provided by any one or more of the above pathways, a metabolic pathway for the production of FAMEs from any such fatty acids utilizes the activity of a fatty acid O-methyltransferase, as shown in FIG. 3 (which includes specific information about exemplary, non-limiting enzymes). The enzymatic conversion of a free fatty acid to a FAME is achieved through the action of a fatty acid O-methyltransferase. This enzyme uses S-adenosylmethionine (SAM) as the methyl donor. The energy to drive the production of SAM is derived from the hydrolysis of ATP by a methionine kinase (such as the noted methionine adenosyl-transferase) and also by the irreversible hydrolysis of S-adenosyl-homocysteine (SAHC) to adenosine and homocysteine. As depicted in FIG. 3, the latter may be converted to methionine, which may be utilized as a substrate in the production of another S-adenosylmethionine (SAM) molecule.

The proposed pathway of some variations employs genetic modifications in addition to the expression of the fatty acid O-methyltransferase. Standard methodologies (known in the art and further described herein) can be used to generate needed gene expression (or gene disruptions, as described elsewhere herein). In some embodiments, the following enzymatic activities are expressed in C. necator, such as along with expression of a suitable fatty acid O-methyltransferase: phosphoglucose isomerase, inositol-1-phosphate synthase, inositol monophosphatase, myo-inositol dehydrogenase, myo-inosose-2-dehydratase, inositol 2-dehydrogenase, deoxy-D-gluconate isomerase, 5-dehydro-2-deoxygluconokinase, deoxyphosphogluconate aldolase, aldehyde dehydrogenase, malonyl-CoA synthetase, fatty acid synthase (which may be any suitable complex), fatty acyl-CoA/ACP thioesteraseadenosine hydrolase (e.g., ribonucleoside hydrolase 3), and S-adenosyl-homocysteine hydrolase. One or more of these expressed enzymatic activities may be expressed from heterologous (including exogenous) nucleic acid sequences. In various embodiments, the following genes can be employed to encode suitable enzymes to achieve desired levels of expression: E. coli pgi, suhB, aldA, tesA, netE, metK and rihC, S. cerevisiae ino-1, B. subtilis iolG, iolE, iolD, iolB, iolC, and iolJ, R. norvegicus Ahcy, R. japonicum matB, and matB of Rhizobium leguminosum. In various other embodiments, any combination of these genes, and those described in the following paragraphs, and/or functional variants of these, may be provided or employed in a microorganism cell or culture, so as to have the enzymatic activities numbered in FIG. 1 and described in Table 1.

For example, FIG. 2 shows homologues of most of the proteins of Table 1, steps 1 to 11 inclusive, in the species C. necator and O. carboxidovorans. These homolog sequences are candidates for use and/or further modification so as to obtain a desired enzymatic conversion indicated in FIG. 1 and Table 1 for the indicated steps. Modifications to achieve a suitable activity and a suitable specificity may be made such as by approaches described herein.

Also, as noted, the fatty acid synthase step indicated in FIG. 1 may be any suitable complex or group of enzymes that function, collectively as a fatty acid synthase (step 12 of FIG. 1). For instance, in E. coli, the fatty acid elongation, step 12, involves the enzymes (encoded by respective genes) FabD, FabH, FabG, FabZ, FabI or FabK, and FabF and FabB. FIG. 4 depicts fatty acid elongation with these enzymes (noting however that the FabH reaction is not depicted, nor is FabK shown). These are exemplary and not meant to be limiting of which fatty acid synthase function may be present, or provided, in a microorganism of the present invention. Also, for these and other sequences provided herein, in some cases further processing occurs before complex formation and/or functionality; nonetheless the sequences provided are indicative of what may be supplied to a particular microorganism.

Also, as far as terminology differences, it is recognized that what is identified herein as “fatty acid synthase,” fatty acid synthase complex,” and the like, for which specific examples and lists are provided, may alternatively be identified as “fatty acid synthase (cyclic elongation, saturated) complex.” It is intended that the latter term does not include the enzyme malonyl-CoA ACP transacylase. It also is intended that the terms “fatty acid synthase” and “fatty acid synthase complex” may include analogous pathways in microorganisms that do not share the particular listed enzymes.

Further, and more generally as may be used herein, fatty acid “enzyme” means any enzyme involved in fatty acid biosynthesis. Fatty acid enzymes can be expressed or overexpressed in host cells to produce fatty acids. Non-limiting examples of fatty acid enzymes include fatty acid synthases and thioesterases. A number of these enzymes, as well as other useful enzymes for making the products described herein, have been disclosed in, for example, International Patent Application Nos. PCT/US2010/030655, PCT/US2007/011923 and PCT/US2008/058788, which are incorporated herein by reference for their teaching of such enzymes.

As may be used herein, the term “fatty acid derivative” means products made in part from the fatty acid biosynthetic pathway of the production host organism. “Fatty acid derivative” also includes products made in part from acyl-ACP or acyl-ACP derivatives. The fatty acid biosynthetic pathway includes fatty acid synthase enzymes which can be engineered to produce fatty acid derivatives, and in some examples can be expressed with additional enzymes to produce fatty acid derivatives having desired carbon chain characteristics. Exemplary fatty acid derivatives include for example, fatty acids, acyl-CoAs, fatty aldehydes, short and long chain alcohols, hydrocarbons, fatty alcohols, ketones, and esters (e.g., waxes, fatty acid esters, or fatty esters). Examples of such fatty acid derivative pathways, enzymes and derivatives may be found in International Patent Application Nos. PCT/US2010/030655, which is incorporated herein by reference for their teaching of such fatty acid derivative pathways, enzymes and derivatives.

Also as may be used herein, the term “fatty acid derivative enzymes” means all enzymes that may be expressed or overexpressed in the production of fatty acid derivatives. These enzymes are collectively referred to herein as fatty acid derivative enzymes. These enzymes may be part of the fatty acid biosynthetic pathway. Non-limiting examples of fatty acid derivative enzymes include fatty acid synthases, thioesterases, acyl-CoA synthases, acyl-CoA reductases, alcohol dehydrogenases, alcohol acyl transferases, carboxylic acid reductases, fatty alcohol-forming acyl-CoA reductase, ester synthases, aldehyde biosynthetic polypeptides, and alkane biosynthetic polypeptides. Fatty acid derivative enzymes convert a substrate into a fatty acid derivative. In some examples, the substrate may be a fatty acid derivative which the fatty acid derivative enzyme converts into a different fatty acid derivative. A number of these enzymes, as well as other useful enzymes for making the products described herein, have been disclosed in, for example, International Patent Application Nos. PCT/US2010/030655, PCT/US2007/011923 and PCT/US2008/058788, which are incorporated herein by reference for their teaching of such enzymes.

As used herein, the term “fatty acid degradation enzyme” means an enzyme involved in the breakdown or conversion of a fatty acid or fatty acid derivative into another product. A nonlimiting example of a fatty acid degradation enzyme is an acyl-CoA synthase. A number of these enzymes, as well as other useful enzymes for making the products described herein, have been disclosed in, for example, PCT/US2010/030655, PCT/US2007/011923 and PCT/US2008/058788, which are incorporated herein by reference for their teaching of such enzymes. Additional examples of fatty acid degradation enzymes are described herein.

Accordingly, based on the teachings herein and in these incorporated references, embodiments of the present invention include compositions (e.g., microorganisms, culture systems, etc.) and methods that include the specific approaches to fatty acid production and FAME production described herein, and also additional fatty acid derivative products, methods of making these, and the above-recited fatty acid derivative enzymes. In many embodiments, the net flux though a selected fatty acid biosynthesis pathway is achieved; this may involve modifications of one or more fatty acid enzymes (such as those listed in step 12 of Table 1). As to fatty acid degradation enzymes, in various embodiments their activity may be decreased so as to increase net production efficiency of a desired product.

Also, for all nucleic acid and amino acid sequences provided herein, it is appreciated that conservatively modified variants of these sequences are included, and are within the scope of the invention in its various embodiments. Functionally equivalent nucleic acid and amino acid sequences (functional variants), which may include conservatively modified variants as well as more extensively varied sequences, which are well within the skill of the person of ordinary skill in the art, and microorganisms comprising these, also are within the scope of various embodiments of the invention, as are methods and systems comprising such sequences and/or microorganisms. Also, as used herein, the language “sufficiently homologous” refers to proteins or portions thereof that have amino acid sequences that include a minimum number of identical or equivalent amino acid residues when compared to an amino acid sequence of the amino acid sequences listed in Table 1 such that the protein or portion thereof is able to participate in the respective reaction shown in FIG. 1 and described in Table 1. To determine whether a particular protein or portion thereof is sufficiently homologous may be determined by an assay of enzymatic activity, such as those commonly known in the art. In various embodiments, nucleic acid sequences encoding sufficiently homologous proteins or portions thereof are within the scope of the invention. More generally, nucleic acids sequences that encode a particular amino acid sequence employed in the invention may vary due to the degeneracy of the genetic code, and nonetheless fall within the scope of the invention. Table 2 provides a summary of similarities among amino acids, upon which conservative and less conservative substitutions may be based, and also various codon redundancies that reflect this degeneracy.

In some embodiments, the aldehyde dehydrogenase is encoded by the aldA gene of E. coli. This gene has been shown to encode an enzyme capable of the dehydrogenation of malonate semialdehyde to produce malonate. The Ahcy gene from Rattus norvegicus has been shown to encode a large 5074 amino acid protein that possesses S-adenosylhomocysteine hydrolase activity. The protein can be readily expressed actively in E. coli.

Also, the S-adenosylmethionine-dependant methyltransferases that catalyze the methyl transfer to form a FAME, as depicted in FIG. 3, may be from or may be a mutated/selected variant of such enzymes reported to catalyze formation of branched fatty acids in the study of insect hormones. These enzymes can be classified as Juvenile hormone (JH) acid O-methyltransferases. Recently a Juvenile hormone (JH) acid O-methyltransferases from D. melanogaster has been purified (Niwa et al., “Juvenile hormone acid O-methyltransferase in Drosophila melanogaster,” Insect Biochemistry and Molecular Biology Volume 38, Issue 7, July 2008, pp. 714-720). It was shown that these O-methyltransferases are active on fatty acids including palmitate, although at less than 1% of the activity of the natural substrates.

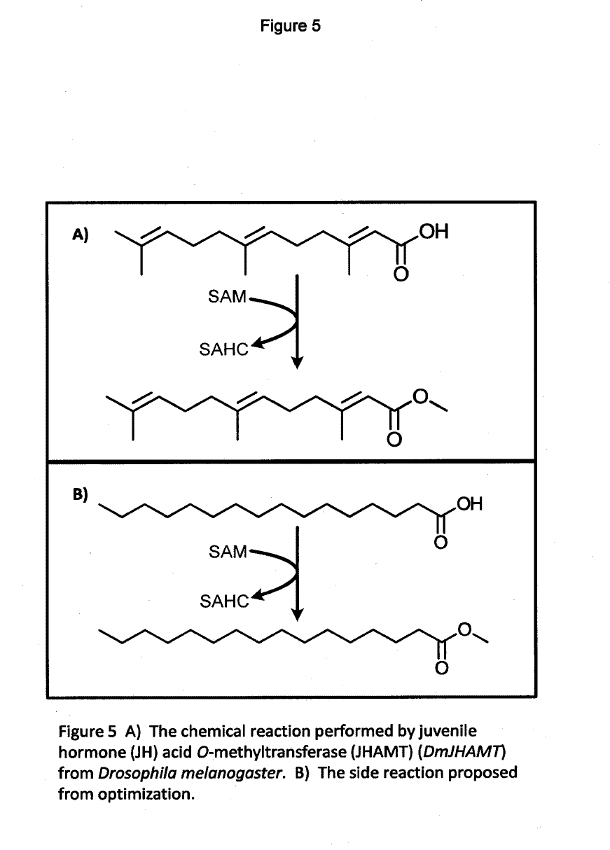

Accordingly, in some embodiments of the invention, S-adenosylmethionine-dependant methyltransferases (such as Juvenile hormone (JH) acid O-methyltransferases, JHAMT), and functionally equivalent or evolved variants thereof, are active for conversion of saturated fatty acids to saturated fatty acid methyl esters, i.e. for fatty acids not containing carbon-carbon double bonds. Example 1 provides an exemplary prophetic example that may lead to obtaining a suitable functional equivalent or evolved variant of a JH O-methyltransferase, and FIG. 5 depicts the chemical reactions A) performed by JHAMT from D. melanogaster and B) the side reaction the increased activity and specificity of which are goals of Example 1 and similar approaches to enzyme evolution. In various embodiments, a functional variant demonstrates activity for such side reaction, forming a methyl ester of a fatty acid that is completely or largely saturated, that is at least 10, 20, 30, 40, or at least 50 percent greater than the activity for such side reaction as D. melanogaster JHAMT.

Other O-methyltransferases may provide desired functionality in their native states, and/or after suitable modification such as described herein. Candidate methyltransferase proteins are provided in the following table, Table 3, which is not meant to be limiting:

TABLE 3
Source Identification

| | |
|---|---|
| JHAMT Dm (Drosophila melanogaster) | NP_609793.2 GI:24584607 |
| JHAMT tcMTS (Tribolium castaneum) | EFA02917.1 GI:270006469 |
| Putative JHAMT MT1 (Tribolium castaneum) | (GenBank AB360761) |
| Putative JHAMT tcMT2 (Tribolium castaneum) | (GenBank AB360762) |
| Mycobacterium smegmatis, str. MC2 155, | gb\|ABK74306.1\| GI:118173410 |
| methyltransferase | YP_884514.1 GI:118472123 |
| Cancer pagurus (edible crab) putative farnesoic acid O- | AAR00732.1 GI:37702161 |
| methyltransferase | |
| JHAMT Shrimp (Metapenaeus ensis). AF333042 | (Y. I. N. Silva Gunawardene et. al., Function and |
| | cellular localization of farnesoic acid O- |
| | methyltransferase (FAMeT) in the shrimp, Metapenaeus |
| | ensis, Eur. J. Biochem. 269, 3587-3595, 2002) |
| Ralstonia solanacearum UW5551 PhcB | ZP_00943805.1 GI:83746757 |

The last candidate protein also is provided as SEQ ID NO:033, also provided below:

| | |
|---|---|
| 1 | myspnqidpa vsfrnsqgqq vrgtiitlqr ralvmevynp ysivqvsevl sdlaikmgtr |
| 61 | qaylgkavvv slvntgltav vsvtlteewr gladvqdspk lvgeearafv qdweerfrir |
| 121 | hdygivvnem raflaevsrw veqvdlsdsl pkegenrlrl dvfqelaepi tlkvkyfqdw |
| 181 | leskaadvep elapahrsfa qsalhplllr apfvyrtftk plgyagdyem vnqiisdpre |
| 241 | gpstyfqivn atflnaavar ahrnrieilv qylsdlatqa laagrqfkvl nvgcgpavei |
| 301 | qrfihqhpep qqlafqlvdf seetldytrr qmdnvrhatn knvdiefvhe svhqllkrrv |
| 361 | gpdspemgef davycaglfd ylsdkvcnrl lthfaartrk ggtllvtnvh gsnpeklsme |
| 421 | hllewylvyr dearmesllp agsanvrlft ddtgvnvfaq arvgdhv. |

More generally, the invention encompasses various genetic modifications and evaluations to certain microorganisms. The scope of the invention is not meant to be limited to such microorganism species, but to be generally applicable to a wide range of suitable microorganisms. As the genomes of various species become known, features of the present invention easily may be applied to an ever-increasing range of suitable microorganisms. Further, given the relatively low cost of genetic sequencing, the genetic sequence of a species of interest may readily be determined to make application of aspects of the present invention more readily obtainable (based on the ease of application of genetic modifications to an organism having a known genomic sequence). More generally, a microorganism used for the present invention may be selected from bacteria, cyanobacteria, filamentous fungi, and yeasts.

More particularly, based on the various criteria described herein, suitable microbial hosts for the bio-production of FAMEs provided herein generally may include, but are not limited to, any gram negative organisms such as E. coli, Oligotropha carboxidovorans, or Pseudomononas sp.; any gram positive microorganism, for example Bacillus subtilis, Lactobaccilus sp. or Lactococcus sp.; any yeast, for example Saccharomyces cerevisiae, Pichia pastoris or Pichia stipitis; and other groups of microbial species. Species and other phylogenic identifications herein are according to the classification known to a person skilled in the art of microbiology.

More particularly, suitable microbial hosts for the bio-production of FAMEs generally include, but are not limited to, members of the genera Clostridium, Zymomonas, Escherichia, Salmonella, Rhodococcus, Pseudomonas, Bacillus, Lactobacillus, Enterococcus, Alcaligenes, Klebsiella, Paenibacillus, Arthrobacter, Corynebacterium, Brevibacterium, Pichia, Candida, Hansenula and Saccharomyces.

Hosts that may be particularly of interest include: Oligotropha carboxidovorans (such as strain OM5), Escherichia coli, Alcaligenes eutrophus (Cupriavidus necator), Bacillus licheniformis, Paenibacillus macerans, Rhodococcus erythropolis, Pseudomonas putida, Lactobacillus plantarum, Enterococcus faecium, Enterococcus gallinarium, Enterococcus faecalis, Bacillus subtilis and Saccharomyces cerevisiae.

In some embodiments, the recombinant microorganism is a gram-negative bacterium. In some embodiments, the recombinant microorganism is selected from the genera Zymomonas, Escherichia, Pseudomonas, Alcaligenes, and Klebsiella. In some embodiments, the recombinant microorganism is selected from the species Escherichia coli, Cupriavidus necator, Oligotropha carboxidovorans, and Pseudomonas putida. In some embodiments, the recombinant microorganism is an E. coli strain.

In some embodiments, the recombinant microorganism is a gram-positive bacterium. In some embodiments, the recombinant microorganism is selected from the genera Clostridium, Salmonella, Rhodococcus, Bacillus, Lactobacillus, Enterococcus, Paenibacillus, Arthrobacter, Corynebacterium, and Brevibacterium. In some embodiments, the recombinant microorganism is selected from the species Bacillus licheniformis, Paenibacillus macerans, Rhodococcus erythropolis, Lactobacillus plantarum, Enterococcus faecium, Enterococcus gallinarium, Enterococcus faecalis, and Bacillus subtilis. In particular embodiments, the recombinant microorganism is a B. subtilis strain.

In some embodiments, the recombinant microorganism is a yeast. In some embodiments, the recombinant microorganism is selected from the genera Pichia, Candida, Hansenula and Saccharomyces. In particular embodiments, the recombinant microorganism is Saccharomyces cerevisiae.

Also, in some embodiments the microorganism comprises an endogenous fatty acid and/or fatty acid methyl ester production pathways (which may, in some such embodiments, be enhanced), whereas in other embodiments the microorganism does not comprise one or either of these production pathways, but is provided with one or more nucleic acid sequences encoding polypeptides having enzymatic activity or activities to complete a pathway, described herein, resulting in production of FAMEs. In some embodiments, the particular sequences disclosed herein, or conservatively modified variants thereof, are provided to a selected microorganism, such as selected from one or more of the species and groups of species or other taxonomic groups listed above.

There are numerous references that teach modulation and modification of fatty acid metabolism. Particular embodiments of the present invention may integrate various such teachings without departing from the present invention. For example, U.S. Patent Publications US2010/0251601, published Oct. 7, 2010, and US2010/0249470, published Sep. 30, 2010, are incorporated by reference herein for their teachings, particularly their teachings of genes, modified genes, and resultant proteins that may be used to modify fatty acid metabolism, and also their teachings of fatty acid derivatives (particularly those that may be made in concert with the present invention). FIG. 40 of the '470 publication is specifically incorporated by reference herein.

In particular, the referenced FIG. 40 catalogues many modifications, such as to increase or decrease enzymatic activity, of many gene and proteins that are involved with synthesis of fatty acids and derivatives of fatty acids. These may be employed in combination with other teachings of the present application. Also, based in part on these teachings, numerous alternatives may be employed for the various genes and proteins represented in steps 12 and 13.

Notwithstanding the discussion on the use of such chemolithotrophs and syngas components for carbon and energy sources, pathways and polynucleotides encoding polypeptides exhibiting enzymatic activity of such pathways described herein also may be used (introduced) in species, methods and systems that use sugars or other suitable substrates as the carbon and energy source.

Suitable substrates include glucose, fructose, xylose, arabinose, and sucrose, as well as mixtures of any of these sugars. Sucrose may be obtained from feedstocks such as sugar cane, sugar beets, cassava, and sweet sorghum. Glucose and dextrose may be obtained through saccharification of starch-based feedstocks including grains such as corn, wheat, rye, barley, and oats. Xylose and arabinose may be obtained from processing of cellulosic materials.

Suitable substrates may generally include, but are not limited to, monosaccharides such as glucose and fructose, oligosaccharides such as lactose or sucrose, polysaccharides such as starch or cellulose or mixtures thereof and unpurified mixtures from renewable feedstocks such as cheese whey permeate, cornsteep liquor, sugar beet molasses, and barley malt. In addition, methylotrophic organisms are known to utilize a number of other carbon containing compounds such as methylamine, glucosamine and a variety of amino acids for metabolic activity. For example, methylotrophic yeast are known to utilize the carbon from methylamine to form trehalose or glycerol (Bellion et al., Microb. Growth C1 Compd. [Int. Symp.], 7th (1993), 415-32. Editor(s): Murrell, J. Collin; Kelly, Don P. Publisher: Intercept, Andover, UK). Similarly, various species of Candida will metabolize alanine or oleic acid (Sulter et al., Arch. Microbiol. 153:485-489 (1990)). Hence it is contemplated that the source of carbon utilized in embodiments of the present invention may encompass a wide variety of carbon-containing substrates, particularly in combination with syngas components.

In addition, fermentable sugars may be obtained from cellulosic and lignocellulosic biomass through processes of pretreatment and saccharification, as described, for example, in U.S. Patent App. Pub. No. US20070031918A1, which is incorporated by reference herein for its teachings. Biomass refers to any cellulosic or lignocellulosic material and includes materials comprising cellulose, and optionally further comprising hemicellulose, lignin, starch, oligosaccharides and/or monosaccharides. Biomass may also comprise additional components, such as proteins and/or lipids. Biomass may be derived from a single source, or biomass can comprise a mixture derived from more than one source; for example, biomass could comprise a mixture of corn cobs and corn stover, or a mixture of grass and leaves. Biomass includes, but is not limited to, bioenergy crops, agricultural residues, municipal solid waste, industrial solid waste, sludge from paper manufacture, yard waste, wood and forestry waste. Examples of biomass include, but are not limited to, corn grain, corn cobs, crop residues such as corn husks, corn stover, grasses, wheat, wheat straw, barley, barley straw, hay, rice straw, switchgrass, waste paper, sugar cane bagasse, sorghum, soy, components obtained from milling of grains, trees, branches, roots, leaves, wood chips, sawdust, shrubs and bushes, vegetables, fruits, flowers and animal manure. Any such biomass may be used in a bio-production method or system to provide a carbon source.

The ability to genetically modify a host cell is essential for the production of any genetically modified (recombinant) microorganism. The mode of gene transfer technology may be by electroporation, conjugation, transduction, or natural transformation. A broad range of host conjugative plasmids and drug resistance markers are available. The cloning vectors are tailored to the host organisms based on the nature of antibiotic resistance markers that can function in that host.

For various embodiments of the invention the genetic manipulations may be described to include various genetic manipulations, including those directed to change regulation of, and therefore ultimate activity of, an enzyme or enzymatic activity of an enzyme identified in any of the respective pathways. Such genetic modifications may be directed to transcriptional, translational, and post-translational modifications that result in a change of enzyme activity and/or selectivity under selected and/or identified culture conditions and/or to provision of additional nucleic acid sequences such as to increase copy number and/or mutants of an enzyme related to FAME production. Specific methodologies and approaches to achieve such genetic modification are well known to one skilled in the art, and include, but are not limited to: increasing expression of an endogenous genetic element; decreasing functionality of a repressor gene; introducing a heterologous genetic element; increasing copy number of a nucleic acid sequence encoding a polypeptide catalyzing an enzymatic conversion step to produce a FAME; mutating a genetic element to provide a mutated protein to increase specific enzymatic activity; over-expressing; under-expressing; over-expressing a chaperone; knocking out a protease; altering or modifying feedback inhibition; providing an enzyme variant comprising one or more of an impaired binding site for a repressor and/or competitive inhibitor; knocking out a repressor gene; evolution, selection and/or other approaches to improve mRNA stability as well as use of plasmids having an effective copy number and promoters to achieve an effective level of improvement. Random mutagenesis may be practiced to provide genetic modifications that may fall into any of these or other stated approaches. The genetic modifications further broadly fall into additions (including insertions), deletions (such as by a mutation) and substitutions of one or more nucleic acids in a nucleic acid of interest. In various embodiments a genetic modification results in improved enzymatic specific activity and/or turnover number of an enzyme. Without being limited, changes may be measured by one or more of the following: K_{M}; Kₑₐₜ; and K_{avidity}.

In various embodiments, to function more efficiently, a microorganism may comprise one or more gene deletions. For example, in E. coli, the genes encoding the pyruvate kinase (pfkA and pfkB), lactate dehydrogenase (ldhA), phosphate acetyltransferase (pta), pyruvate oxidase (poxB), and pyruvate-formate lyase (pflB) may be disrupted, including deleted. Such gene disruptions, including deletions, are not meant to be limiting, and may be implemented in various combinations in various embodiments.

Gene deletions may be accomplished by mutational gene deletion approaches, and/or starting with a mutant strain having reduced or no expression of one or more of these enzymes, and/or other methods known to those skilled in the art. Gene deletions may be effectuated by any of a number of known specific methodologies, including but not limited to the RED/ET methods using kits and other reagents sold by Gene Bridges (Gene Bridges GmbH, Dresden, Germany, www.genebridges.com). The homologous recombination method using Red/ET recombination, is known to those of ordinary skill in the art and described in U.S. Pat. Nos. 6,355,412 and 6,509,156, issued to Stewart et al. and incorporated by reference herein for its teachings of this method. Material and kits for such method are available from Gene Bridges (Gene Bridges GmbH, Heidelberg (formerly Dresden), Germany, <<www.genebridges.com>>), and the method proceeded by following the manufacturer's instructions. The method replaces the target gene by a selectable marker via homologous recombination performed by the recombinase from λ-phage. The host organism expressing λ-red recombinase is transformed with a linear DNA product coding for a selectable marker flanked by the terminal regions (generally ˜50 bp, and alternatively up to about ˜300 bp) homologous with the target gene or promoter sequence.

Further, for FAME production, such genetic modifications may be chosen and/or selected for to achieve a higher flux rate through certain basic pathways within the respective FAME production pathway and so may affect general cellular metabolism in fundamental and/or major ways. Another method enabling genetic modification of chromosomal DNA including gene deletion in C. necator involves integration of counterselectable markers, such as Bacillus sacB markers which confer sensitivity to sucrose, via suicide plasmids. These methods are well known in the art.

As used herein, the term “gene disruption,” or grammatical equivalents thereof (and including “to disrupt enzymatic function,” “disruption of enzymatic function,” and the like), is intended to mean a genetic modification to a microorganism that renders the encoded gene product as having a reduced polypeptide activity compared with polypeptide activity in or from a microorganism cell not so modified. The genetic modification can be, for example, deletion of the entire gene, deletion or other modification of a regulatory sequence required for transcription or translation, deletion of a portion of the gene which results in a truncated gene product (e.g., enzyme) or by any of various mutation strategies that reduces activity (including to no detectable activity level) the encoded gene product. A disruption may broadly include a deletion of all or part of the nucleic acid sequence encoding the enzyme, and also includes, but is not limited to other types of genetic modifications, e.g., introduction of stop codons, frame shift mutations, introduction or removal of portions of the gene, and introduction of a degradation signal, those genetic modifications affecting mRNA transcription levels and/or stability, and altering the promoter or repressor upstream of the gene encoding the enzyme.

In some embodiments, a gene disruption is taken to mean any genetic modification to the DNA, mRNA encoded from the DNA, and the amino acid sequence resulting there from that results in reduced polypeptide activity. Many different methods can be used to make a cell having reduced polypeptide activity. For example, a cell can be engineered to have a disrupted regulatory sequence or polypeptide-encoding sequence using common mutagenesis or knock-out technology. See, e.g., Methods in Yeast Genetics (1997 edition), Adams et al., Cold Spring Harbor Press (1998). One particularly useful method of gene disruption is complete gene deletion because it reduces or eliminates the occurrence of genetic reversions in the genetically modified microorganisms of the invention. Accordingly, a disruption of a gene whose product is an enzyme thereby disrupts enzymatic function. Alternatively, antisense technology can be used to reduce the activity of a particular polypeptide. For example, a cell can be engineered to contain a cDNA that encodes an antisense molecule that prevents a polypeptide from being translated. The term “antisense molecule” as used herein encompasses any nucleic acid molecule or nucleic acid analog (e.g., peptide nucleic acids) that contains a sequence that corresponds to the coding strand of an endogenous polypeptide. An antisense molecule also can have flanking sequences (e.g., regulatory sequences). Thus, antisense molecules can be ribozymes or antisense oligonucleotides. A ribozyme can have any general structure including, without limitation, hairpin, hammerhead, or axhead structures, provided the molecule cleaves RNA. Further, gene silencing can be used to reduce the activity of a particular polypeptide.

The term “reduction” or “to reduce” when used in such phrase and its grammatical equivalents are intended to encompass a complete elimination of such conversion(s). The term “heterologous DNA,” “heterologous nucleic acid sequence,” and the like as used herein refers to a nucleic acid sequence wherein at least one of the following is true: (a) the sequence of nucleic acids is foreign to (i.e., not naturally found in) a given host microorganism; (b) the sequence may be naturally found in a given host microorganism, but in an unnatural (e.g., greater than expected) amount; or (c) the sequence of nucleic acids comprises two or more subsequences that are not found in the same relationship to each other in nature. For example, regarding instance (c), a heterologous nucleic acid sequence that is recombinantly produced will have two or more sequences from unrelated genes arranged to make a new functional nucleic acid. Embodiments of the present invention may result from introduction of an expression vector into a host microorganism, wherein the expression vector contains a nucleic acid sequence coding for an enzyme that is, or is not, normally found in a host microorganism. With reference to the host microorganism's genome prior to the introduction of the heterologous nucleic acid sequence, then, the nucleic acid sequence that codes for the enzyme is heterologous (whether or not the heterologous nucleic acid sequence is introduced into that genome). Also, when the genetic modification of a gene product, i.e., an enzyme, is referred to herein, including the claims, it is understood that the genetic modification is of a nucleic acid sequence, such as or including the gene, that normally encodes the stated gene product, i.e., the enzyme. The term “heterologous” is intended to include the term “exogenous” as the latter term is generally used in the art.

Bio-production media, which is used in embodiments of the present invention with genetically modified microorganisms, must contain suitable carbon substrates for the intended metabolic pathways. As described hereinbefore, suitable carbon substrates include carbon monoxide, carbon dioxide, and various monomeric and oligomeric sugars.

In some variations, one or more carbon sources should be minimized or excluded from the bio-production media. In the case of auxotrophic fermentations of C. necator, minimal medias may be employed, as supplementation of certain carbon sources, particularly amino acids, can cause metabolism of these compounds rather than hydrogen and carbon dioxide. Also, it is known in the art that syngas streams may contain toxic components such as heavy metals and aromatic tars. In some embodiments, metals and tars are minimized in the bio-production media.

In some embodiments, genetic elements that provide increased tolerance to, or detoxify, tars and similar components are identified and thereafter incorporated into a microorganism of interest for biodiesel production. One technique that may precisely and rapidly identify such genomic elements is the SCALES technique, described in U.S. Patent Publication US2006/0084098, published Apr. 20, 2006, and incorporated by reference herein for the teachings of the technique of that application. Inter alia, this technique may be applied to identify genetic elements that provide increased tolerance to toxic components associated with a particular syngas from a particular source, or may be applied more broadly.

Typically cells are grown at a temperature in the range of about 25° C. to about 40° C. in an appropriate medium, as well as up to 70° C. for thermophilic microorganisms. Suitable growth media for embodiments of the present invention are common commercially prepared media such as Luria Bertani (LB) broth, M9 minimal media, Sabouraud Dextrose (SD) broth, Yeast medium (YM) broth (Ymin) yeast synthetic minimal media and minimal media as described herein, such as M9 minimal media. Other defined or synthetic growth media may also be used, and the appropriate medium for growth of the particular microorganism will be known by one skilled in the art of microbiology or bio-production science. In various embodiments a minimal media may be developed and used that does not comprise, or that has a low level of addition (e.g., less than 0.2, or less than one, or less than 0.05 percent) of one or more of yeast extract and/or a complex derivative of a yeast extract, e.g., peptone, tryptone, etc.

Suitable pH ranges for the bio-production are between pH 3.0 to pH 10.0, where pH 6.0 to pH 8.0 is a typical pH range for the initial condition. However, the actual culture conditions for a particular embodiment are not meant to be limited by these pH ranges.

Bio-productions may be performed under aerobic, microaerobic, or anaerobic conditions, with or without agitation. The operation of cultures and populations of microorganisms to achieve aerobic, microaerobic and anaerobic conditions are known in the art, and dissolved oxygen levels of a liquid culture comprising a nutrient media and such microorganism populations may be monitored to maintain or confirm a desired aerobic, microaerobic or anaerobic condition. When syngas is used as a feedstock, aerobic conditions may be utilized (although not required to practice this invention). When sugars are used, anaerobic, aerobic or microaerobic conditions can be implemented in various embodiments.

The amount of FAMEs produced in a bio-production media generally can be determined using a number of methods known in the art, for example, high performance liquid chromatography (HPLC), gas chromatography (GC), or GC/Mass Spectroscopy (MS).

Any of the recombinant microorganisms as described and/or referred to above may be introduced into an industrial bio-production system where the microorganisms convert a carbon source into biodiesel in a commercially viable operation. The bio-production system includes the introduction of such a recombinant microorganism into a bioreactor vessel, with a carbon source substrate and bio-production media suitable for growing the recombinant microorganism, and maintaining the bio-production system within a suitable temperature range (and dissolved oxygen concentration range if the reaction is aerobic or microaerobic) for a suitable time to obtain a desired conversion of a portion of the substrate molecules to FAMEs. Industrial bio-production systems and their operation are well-known to those skilled in the arts of chemical engineering and bioprocess engineering. The following paragraphs provide an overview of the methods and aspects of industrial systems that may be used for the bio-production of FAMEs as biodiesel constituents.

In various embodiments, syngas components or sugars are provided to a microorganism, such as in an industrial system comprising a reactor vessel in which a defined media (such as a minimal salts media including but not limited to M9 minimal media, potassium sulfate minimal media, yeast synthetic minimal media and many others or variations of these), an inoculum of a microorganism providing an embodiment of the biosynthetic pathway(s) taught herein, and the carbon source may be combined. The carbon source enters the cell and is catabolized by well-known and common metabolic pathways to yield common metabolic intermediates, including phosphoenolpyruvate (PEP). (See Molecular Biology of the Cell, 3^{rd} Ed., B. Alberts et al. Garland Publishing, New York, 1994, pp. 42-45, 66-74, incorporated by reference for the teachings of basic metabolic catabolic pathways for sugars; Principles of Biochemistry, 3^{rd} Ed., D. L. Nelson & M. M. Cox, Worth Publishers, New York, 2000, pp. 527-658, incorporated by reference for the teachings of major metabolic pathways; and Biochemistry, 4^{th} Ed., L. Stryer, W. H. Freeman and Co., New York, 1995, pp. 463-650, also incorporated by reference for the teachings of major metabolic pathways.).

Further to types of industrial bio-production, various embodiments of the present invention may employ a batch type of industrial bioreactor. A classical batch bioreactor system is considered “closed” meaning that the composition of the medium is established at the beginning of a respective bio-production event and not subject to artificial alterations and additions during the time period ending substantially with the end of the bio-production event. Thus, at the beginning of the bio-production event the medium is inoculated with the desired organism or organisms, and bio-production is permitted to occur without adding anything to the system. Typically, however, a “batch” type of bio-production event is batch with respect to the addition of carbon source and attempts are often made at controlling factors such as pH and oxygen concentration. In batch systems the metabolite and biomass compositions of the system change constantly up to the time the bio-production event is stopped. Within batch cultures cells moderate through a static lag phase to a high growth log phase and finally to a stationary phase where growth rate is diminished or halted. If untreated, cells in the stationary phase will eventually die. Cells in log phase generally are responsible for the bulk of production of a desired end product or intermediate.

A variation on the standard batch system is the fed-batch system. Fed-batch bio-production processes are also suitable when practicing embodiments of the present invention and comprise a typical batch system with the exception that the nutrients, including the substrate, are added in increments as the bio-production progresses. Fed-batch systems are useful when catabolite repression is apt to inhibit the metabolism of the cells and where it is desirable to have limited amounts of substrate in the media. Measurement of the actual nutrient concentration in fed-batch systems may be measured directly, such as by sample analysis at different times, or estimated on the basis of the changes of measurable factors such as pH, dissolved oxygen and the partial pressure of waste gases such as CO₂. Batch and fed-batch approaches are common and well known in the art and examples may be found in Thomas D. Brock in Biotechnology: A Textbook of Industrial Microbiology, Second Edition (1989) Sinauer Associates, Inc., Sunderland, Mass., Deshpande, Mukund V., Appl. Biochem. Biotechnol., 36:227, (1992), and Biochemical Engineering Fundamentals, 2^{nd} Ed. J. E. Bailey and D. F. Ollis, McGraw Hill, New York, 1986, herein incorporated by reference for general instruction on bio-production, which as used herein may be aerobic, microaerobic, or anaerobic.

Although embodiments of the present invention may be performed in batch mode, or in fed-batch mode, it is contemplated that the invention would be adaptable to continuous bio-production methods. Continuous bio-production is considered an “open” system where a defined bio-production medium is added continuously to a bioreactor and an equal amount of conditioned media is removed simultaneously for processing. Continuous bio-production generally maintains the cultures within a controlled density range where cells are primarily in log phase growth. Two types of continuous bioreactor operation include a chemostat, wherein fresh media is fed to the vessel while simultaneously removing an equal rate of the vessel contents. The limitation of this approach is that cells are lost and high cell density generally is not achievable. In fact, typically one can obtain much higher cell density with a fed-batch process. Another continuous bioreactor utilizes perfusion culture, which is similar to the chemostat approach except that the stream that is removed from the vessel is subjected to a separation technique which recycles viable cells back to the vessel. This type of continuous bioreactor operation has been shown to yield significantly higher cell densities than fed-batch and can be operated continuously. Continuous bio-production is particularly advantageous for industrial operations because it has less down time associated with draining, cleaning and preparing the equipment for the next bio-production event. Furthermore, it is typically more economical to continuously operate downstream unit operations, such as distillation, than to run them in batch mode.

Continuous bio-production allows for the modulation of one factor or any number of factors that affect cell growth or end product concentration. For example, one method will maintain a limiting nutrient such as the carbon source or nitrogen level at a fixed rate and allow all other parameters to moderate. In other systems a number of factors affecting growth can be altered continuously while the cell concentration, measured by media turbidity, is kept constant. Methods of modulating nutrients and growth factors for continuous bio-production processes as well as techniques for maximizing the rate of product formation are well known in the art of industrial microbiology and a variety of methods are detailed by Brock, supra.

It is contemplated that cells may be immobilized on an inert scaffold as whole cell catalysts and subjected to suitable bio-production conditions for FAME production. Thus, embodiments used in such processes, and in bio-production systems using these processes, include a population of genetically modified microorganisms of the present invention, and a culture system comprising such population in a media comprising nutrients for the population.

The FAME molecules from any such bio-production may be further processed (i.e., recovered, purified, and optionally blended), including to conform to commercial grade quality standards for diesel fuel oils and heating oils, such as those of the ASTM or ANP. Meeting governmental environmental standards, such as from the U.S. Environmental Protection Agency, may also be met given the lack of contaminants often encountered from many petroleum-sourced diesel fuel oil molecules.

The following published resources are incorporated by reference herein for their respective teachings to indicate the level of skill in these relevant arts, and as needed to support a disclosure that teaches how to make and use methods of industrial bio-production of biodiesel, and also industrial systems that may be used to achieve such conversion with any of the recombinant microorganisms of the present invention (Biochemical Engineering Fundamentals, 2^{nd} Ed. J. E. Bailey and D. F. Ollis, McGraw Hill, New York, 1986, entire book for purposes indicated and Chapter 9, pp. 533-657 in particular for biological reactor design; Unit Operations of Chemical Engineering, 5^{th} Ed., W L. McCabe et al., McGraw Hill, New York 1993, entire book for purposes indicated, and particularly for process and separation technologies analyses; Equilibrium Staged Separations, P. C. Wankat, Prentice Hall, Englewood Cliffs, N.J. USA, 1988, entire book for separation technologies teachings).

Also, the scope of the present invention is not meant to be limited to the exact sequences provided herein. It is appreciated that a range of modifications to nucleic acid and to amino acid sequences may be made and still provide a desired functionality, such as a desired enzymatic activity and specificity. The following discussion is provided describe ranges of variation that may be practiced and still remain within the scope of the present invention.

It has long been recognized in the art that some amino acids in amino acid sequences can be varied without significant effect on the structure or function of proteins. Variants included can constitute deletions, insertions, inversions, repeats, and type substitutions so long as the indicated enzyme activity is not significantly adversely affected.

Examples of properties that provide the bases for conservative and other amino acid substitutions are exemplified in Table 2. Accordingly, one skilled in the art may make numerous substitutions to obtain an amino acid sequence variant that exhibits a desired functionality. BLASTP, CLUSTALP, and other alignment and comparison tools may be used to assess highly conserved regions, to which fewer substitutions may be made (unless directed to alter activity to a selected level, which may require multiple substitutions). More substitutions may be made in regions recognized or believed to not be involved with an active site or other binding or structural motif. In accordance with Table 2, for example, substitutions may be made of one polar uncharged (PU) amino acid for a polar uncharged amino acid of a listed sequence, optionally considering size/molecular weight (i.e., substituting a serine for a threonine). Guidance concerning which amino acid changes are likely to be phenotypically silent can be found, inter alia, in Bowie, J. U., et Al., “Deciphering the Message in Protein Sequences: Tolerance to Amino Acid Substitutions,” Science 247:1306-1310 (1990). This reference is incorporated by reference for such teachings, which are, however, also generally known to those skilled in the art. Recognized conservative amino acid substitutions comprise (substitutable amino acids following each colon of a set): ala:ser; arg:lys; asn:gln or his; asp:glu; cys:ser; gln:asn; glu:asp; gly:pro; his:asn or gln; ile:leu or val; leu:/ile or val; lys: arg or gln or glu; met:leu or ile; phe:met or leu or tyr; ser:thr; thr:ser; trp:tyr; tyr:trp or phe; val:ile or leu.

It is noted that codon preferences and codon usage tables for a particular species can be used to engineer isolated nucleic acid molecules that take advantage of the codon usage preferences of that particular species. For example, the isolated nucleic acid provided herein can be designed to have codons that are preferentially used by a particular organism of interest. Numerous software and sequencing services are available for such codon-optimizing of sequences.

The invention provides polypeptides that contain the entire amino acid sequence of an amino acid sequence listed or otherwise disclosed herein. In addition, the invention provides polypeptides that contain a portion of an amino acid sequence listed or otherwise disclosed herein. For example, the invention provides polypeptides that contain a 15 amino acid sequence identical to any 15 amino acid sequence of an amino acid sequence listed or otherwise disclosed herein including, without limitation, the sequence starting at amino acid residue number 1 and ending at amino acid residue number 15, the sequence starting at amino acid residue number 2 and ending at amino acid residue number 16, the sequence starting at amino acid residue number 3 and ending at amino acid residue number 17, and so forth. It will be appreciated that the invention also provides polypeptides that contain an amino acid sequence that is greater than 15 amino acid residues (e.g., 16, 17, 18, 19, 20, 21, 22, 23, 24, 25, 26, 27, 28, 29, 30 or more amino acid residues) in length and identical to any portion of an amino acid sequence listed or otherwise disclosed herein For example, the invention provides polypeptides that contain a 25 amino acid sequence identical to any 25 amino acid sequence of an amino acid sequence listed or otherwise disclosed herein including, without limitation, the sequence starting at amino acid residue number 1 and ending at amino acid residue number 25, the sequence starting at amino acid residue number 2 and ending at amino acid residue number 26, the sequence starting at amino acid residue number 3 and ending at amino acid residue number 27, and so forth. Additional examples include, without limitation, polypeptides that contain an amino acid sequence that is 50 or more amino acid residues (e.g., 100, 150, 200, 250, 300 or more amino acid residues) in length and identical to any portion of an amino acid sequence listed or otherwise disclosed herein. Further, it is appreciated that, per above, a 15 nucleotide sequence will provide a 5 amino acid sequence, so that the latter, and higher-length amino acid sequences, may be defined by the above-described nucleotide sequence lengths having identity with a sequence provided herein.

In various embodiments polypeptides obtained by the expression of the polynucleotide molecules of the present invention may have at least approximately 50%, 60%, 70%, 80%, 90%, 95%, 96%, 97%, 98%, 99% or 100% identity to one or more amino acid sequences encoded by the genes and/or nucleic acid sequences described herein for the biosynthesis pathways. A truncated respective polypeptide has at least about 90% of the full length of a polypeptide encoded by a nucleic acid sequence encoding the respective native enzyme, and more particularly at least 95% of the full length of a polypeptide encoded by a nucleic acid sequence encoding the respective native enzyme. By a polypeptide having an amino acid sequence at least, for example, 95% “identical” to a reference amino acid sequence of a polypeptide is intended that the amino acid sequence of the claimed polypeptide is identical to the reference sequence except that the claimed polypeptide sequence can include up to five amino acid alterations per each 100 amino acids of the reference amino acid of the polypeptide. In other words, to obtain a polypeptide having an amino acid sequence at least 95% identical to a reference amino acid sequence, up to 5% of the amino acid residues in the reference sequence can be deleted or substituted with another amino acid, or a number of amino acids up to 5% of the total amino acid residues in the reference sequence can be inserted into the reference sequence. These alterations of the reference sequence can occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 50%, 60%, 70%, 80%, 85%, 90%, 92%, 95%, 96%, 97%, 98% or 99% identical to any reference amino acid sequence of any polypeptide described herein (which may correspond with a particular nucleic acid sequence described herein), such particular polypeptide sequence can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence according to the present invention, the parameters are set such that the percentage of identity is calculated over the full length of the reference amino acid sequence and that gaps in identity of up to 5% of the total number of amino acid residues in the reference sequence are allowed.

For example, in a specific embodiment the identity between a reference sequence (query sequence, i.e., a sequence of the present invention) and a subject sequence, also referred to as a global sequence alignment, may be determined using the FASTDB computer program based on the algorithm of Brutlag et al. (Comp. App. Biosci. 6:237-245 (1990)). Particular parameters for a particular embodiment in which identity is narrowly construed, used in a FASTDB amino acid alignment, are: Scoring Scheme=PAM (Percent Accepted Mutations) 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. According to this embodiment, if the subject sequence is shorter than the query sequence due to N- or C-terminal deletions, not because of internal deletions, a manual correction is made to the results to take into consideration the fact that the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are lateral to the N- and C-terminal of the subject sequence, which are not matched (i.e., aligned) with a corresponding subject residue, as a percent of the total bases of the query sequence. A determination of whether a residue is matched (i.e., aligned) is determined by results of the FASTDB sequence alignment. This percentage is then subtracted from the percent identity, calculated by the above FASTDB program using the specified parameters, to arrive at a final percent identity score. This final percent identity score is what is used for the purposes of this embodiment. Only residues to the N- and C-termini of the subject sequence, which are not matched (i.e., aligned) with the query sequence, are considered for the purposes of manually adjusting the percent identity score. That is, only query residue positions outside the farthest N- and C-terminal residues of the subject sequence are considered for this manual correction. For example, a 90 amino acid residue subject sequence is aligned with a 100 residue query sequence to determine percent identity. The deletion occurs at the N-terminus of the subject sequence and therefore, the FASTDB alignment does not show a matching (i.e., alignment) of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched (i.e., aligned) with the query. In this case the percent identity calculated by FASTDB is not manually corrected. Once again, only residue positions outside the N- and C-terminal ends of the subject sequence, as displayed in the FASTDB alignment, which are not matched (i.e., aligned) with the query sequence are manually corrected for.

Also as used herein, the term “homology” refers to the optimal alignment of sequences (either nucleotides or amino acids), which may be conducted by computerized implementations of algorithms. “Homology”, with regard to polynucleotides, for example, may be determined by analysis with BLASTN version 2.0 using the default parameters. “Homology” with respect to polypeptides (i.e., amino acids), may be determined using a program, such as BLASTP version 2.2.2 with the default parameters, which aligns the polypeptides or fragments being compared and determines the extent of amino acid identity or similarity between them. It will be appreciated that amino acid homologues includes conservative substitutions, i.e. those that substitute a given amino acid in a polypeptide by another amino acid of similar characteristics. Typically seen as conservative substitutions are the following replacements: replacements of an aliphatic amino acid such as Ala, Val, Leu and Ile with another aliphatic amino acid; replacement of a Ser with a Thr or vice versa; replacement of an acidic residue such as Asp or Glu with another acidic residue; replacement of a residue bearing an amide group, such as Asn or Gln, with another residue bearing an amide group; exchange of a basic residue such as Lys or Arg with another basic residue; and replacement of an aromatic residue such as Phe or Tyr with another aromatic residue. A polypeptide sequence (i.e., amino acid sequence) or a polynucleotide sequence comprising at least 50% homology to another amino acid sequence or another nucleotide sequence respectively has a homology of 50% or greater than 50%, e.g., 60%, 70%, 80%, 90% or 100%.

The above descriptions and methods for sequence identity and homology are intended to be exemplary and it is recognized that these concepts are well-understood in the art.

Further, it is appreciated that nucleic acid sequences may be varied and still encode an enzyme or other polypeptide exhibiting a desired functionality, and such variations are within the scope of the present invention, as are those and other sequences when directed to production of intermediate products (en route to FAME) and other products of commercial value other than FAME (such as derivatives referenced herein), all of which may be collectively referred to as “products.”. Nucleic acid sequences that encode polypeptides that provide the indicated functions for increased FAME production are considered within the scope of the present invention. These may be further defined by the stringency of hybridization, described below, but this is not meant to be limiting when a function of an encoded polypeptide matches a specified biosynthesis pathway enzyme activity.

Further to nucleic acid sequences, “hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid” or “duplex.” “Hybridization conditions” will typically include salt concentrations of less than about 1M, more usually less than about 500 mM and less than about 200 mM. Hybridization temperatures can be as low as 5° C., but are typically greater than 22° C., more typically greater than about 30° C., and often are in excess of about 37° C. Hybridizations are usually performed under stringent conditions, i.e. conditions under which a probe will hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the Tm for the specific sequence at a defined ionic strength and pH. Exemplary stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see for example, Sambrook and Russell and Anderson “Nucleic Acid Hybridization” 1st Ed., BIOS Scientific Publishers Limited (1999), which is hereby incorporated by reference for hybridization protocols. “Hybridizing specifically to” or “specifically hybridizing to” or like expressions refer to the binding, duplexing, or hybridizing of a molecule substantially to or only to a particular nucleotide sequence or sequences under stringent conditions when that sequence is present in a complex mixture (e.g., total cellular) DNA or RNA.

Accordingly, in yet other embodiments, an isolated nucleic acid molecule of the invention, or a microorganism of the invention, comprises a nucleic acid molecule which is a complement of one of the nucleotide sequences shown herein, such as in Table 1, or a portion thereof. As used herein, the term “complementary” refers to a nucleotide sequence that can hybridize to one of the nucleotide sequences listed in Table 1, the sequences provided in the sequence listing herein, thereby forming a stable duplex.

In one aspect of the invention the identity values in the preceding paragraphs are determined using the parameter set described above for the FASTDB software program. It is recognized that identity may be determined alternatively with other recognized parameter sets, and that different software programs (e.g., Bestfit vs. BLASTp). Thus, identity can be determined in various ways. Further, for all specifically recited sequences herein it is understood that conservatively modified variants thereof are intended to be included within the invention.

Thus, polynucleotide (nucleic acid) sequences and polypeptide (e.g., enzyme) sequences of the present invention may be grouped, or characterized, with reference to percent identity, percent homology, and/or degree of hybridization with, a specified sequence. Further, those skilled in the art will understand that the genetic modifications described herein, with reference to E. coli genes and their respective enzymatic activities, and for certain genes of other species, are not meant to be limiting. Given the complete genome sequencing of a large and increasing number of microorganism species, and the level of skill in the art, one skilled in the art will be able to apply the present teachings and disclosures to numerous other microorganisms of interest for increased production of FAME and other products.

Further to the determination of homologous genes in a selected microorganism species, this may be determined as follows. Using as a starting point a gene disclosed herein, one may conduct a homology search and analysis to obtain a listing of potentially homologous sequences for the selected microorganism species. For this homology approach a local blast (www.ncbi.nlm.nih.gov/Tools/) (blastp) comparison using the E. coli protein encoded by the selected gene is performed using different thresholds and comparing to one or more selected species (www.ncbi.nlm.nih.gov/genomes/lproks.cgi). A suitable E-value is chosen at least in part based on the number of results and the desired ‘tightness’ of the homology, considering the number of later evaluations to identify useful genes. Genes so identified may be evaluated in accordance with the teachings of the present invention. Such gene may encode an enzyme wherein that enzyme's amino acid sequence is within a 50, 60, 70, 80, 90, or 95 percent homology of the selected gene. It is noted that such identified and evaluated nucleic acid and amino acid sequences may also be selected, at least in part, by correspondence with the size of the selected gene.

Thus, using such approaches based on identifying sequences that have a specified homology to sequences disclosed herein (“reference sequences”), nucleic acid and amino acid sequences are identified, and may be evaluated and used in embodiments of the invention, wherein the latter nucleic acid and amino acid sequences fall within a specified percentage of sequence identity.

Also, variants or sequences having substantial identity or homology with the polynucleotides encoding enzymes described herein, and their functional equivalents in other species, may be assessed, and assuming a suitable specific functionality is determined (such as by evaluation of enzymatic activity), utilized in the practice and various embodiments of the present invention. Such sequences can be referred to as variants or modified sequences. That is, a polynucleotide sequence may be modified yet still retain the ability to encode a polypeptide exhibiting a desired enzymatic activity. Such variants or modified sequences are thus equivalents. Generally, the variant or modified sequence may comprise at least about 40 to 60 percent, or about 60 to 80 percent, or about 80 to 90 percent, or about 90 to 95 percent, or over 95 percent, sequence identity with the reference sequence (that sequence used to start the analysis).

Similarly, it is appreciated that the encoded amino acid sequence of the polypeptide exhibiting the enzymatic activity may vary and still retain the desired functionality. This may also be quantified by sequence identity, a term known to and applied by those skilled in the art.

In some embodiments, the invention contemplates a genetically modified (e.g., recombinant) microorganism comprising a heterologous nucleic acid sequence that encodes a polypeptide that is an identified enzymatic functional variant of any of the enzymes of the FAME production pathway disclosed herein, wherein the polypeptide has enzymatic activity and specificity effective to perform the enzymatic reaction of the respective FAME production enzyme, so that the recombinant microorganism exhibits greater FAME production than an appropriate control microorganism lacking such nucleic acid sequence. This also applies to other products described herein. Relevant methods of the invention also are intended to be directed to identifying variants that exhibit a desired enzymatic functionality, and the nucleic acid sequences that encode them.

In accordance with the teachings herein, including the examples, microorganisms are modified to provide increased production of desired organic chemical molecules from the carbon sources carbon dioxide and/or carbon monoxide (which in some embodiments may also comprise more complex carbon sources, such as sugars). In making such modified microorganisms, iterative modifications may be made and evaluated, leading to cells having improved characteristics for such production. The modifications may include additions as well as deletions of genetic material.

For any of the examples herein, the following may be used as starting strains:

DSM 541: Name: Cupriavidus necator Makkar and Casida 1987 DSM No.: 541 Synonyms: Ralstonia eutropha (Davis 1969) Yabuuchi et al. 1996, Wautersia eutropha (Davis 1969) Vaneechoutte et al. 2004, Alcaligenes eutrophus Davis 1969 Information: H. G. Schlegel, H 16 PHB⁻4. (Wautersia eutropha). Mutant from DSM 428, does not form poly-β-hydroxy-butyrate Produces ribonuclease, ribulose-1,5-bisphosphate carboxylase. Chemolithotrophic growth with hydrogen. Single cell protein production.

DSM 542: Name: Cupriavidus necator Makkar and Casida 1987 DSM No.: 542 Synonyms: Ralstonia eutropha (Davis 1969) Yabuuchi et al. 1996, Wautersia eutropha (Davis 1969) Vaneechoutte et al. 2004, Alcaligenes eutrophus Davis 1969 Information: —C. König, H 16 G⁺7. (Wautersia eutropha). Mutant from DSM 428. Chemolithotrophic growth with hydrogen. Constitutive G-6-PDH. (Medium 1 or 81, 30° C.). Medium: 1, 30° C. or medium 81, 30°

DSM 428: Name: Cupriavidus necator Makkar and Casida 1987 DSM No.: 428 Other collection no. ATCC 17699, KCTC 22496, NCIB 10442 Synonyms: Ralstonia eutropha (Davis 1969) Yabuuchi et al. 1996, Wautersia eutropha (Davis 1969) Vaneechoutte et al. 2004, Alcaligenes eutrophus Davis 1969 Information: IMG (Alcaligenes eutrophus) E. Wilde, H 16. (Ralstonia eutropha, Wautersia eutropha). Sludge; Germany (216). Harbours a well-studied megaplasmid, pHG1. Produces PHB. Chemolithotrophic growth with hydrogen. Extensively used in studies on chemolithotrophic growth and hydrogenase activity

Also, in various embodiments an oxygen-tolerant CO dehydrogenase complex may be provided for conversion of carbon monoxide to hydrogen in accordance with the water shift reaction (CO+H₂O->CO₂+H₂). Specific oxygen-tolerant genes that may be employed are known, e.g., see “The structural genes encoding CO dehydrogenase subunits (cox L, M and S) in Pseudomonas carboxydovorans OM5 reside on plasmid pHCG3 and are, with the exception of Streptomyces thermoautotrophicus, conserved in carboxydotrophic bacteria,” Iris Hugendieck and Ortwin Meyer (Archives of Microbiology, Volume 157, Number 3, 301-304, DOI: 10.1007/BF00245166). The C. carboxidovorans protein sequences for CoxL, CoxM, and CoxS are provided as SEQ ID NOs.034, 035 and 036 (CAA57829.1 GI:809566, CAA57827.1 GI:809564, and CAA57828.1 GI:809565, respectively). These additions may be combined with various other embodiments in any combination.

Also, and more generally, in accordance with disclosures, discussions, examples and embodiments herein, there may be employed conventional molecular biology, cellular biology, microbiology, and recombinant DNA techniques within the skill of the art. Such techniques are explained fully in the literature. (See, e.g., Sambrook and Russell, “Molecular Cloning: A Laboratory Manual,” Third Edition 2001 (volumes 1-3), Cold Spring Harbor Laboratory Press, Cold Spring Harbor, N.Y.; Animal Cell Culture, R. I. Freshney, ed., 1986). These published resources are incorporated by reference herein for their respective teachings of standard laboratory methods found therein. Such incorporation, at a minimum, is for the specific teaching and/or other purpose that may be noted when citing the reference herein. If a specific teaching and/or other purpose is not so noted, then the published resource is specifically incorporated for the teaching(s) indicated by one or more of the title, abstract, and/or summary of the reference. If no such specifically identified teaching and/or other purpose may be so relevant, then the published resource is incorporated in order to more fully describe the state of the art to which the present invention pertains, and/or to provide such teachings as are generally known to those skilled in the art, as may be applicable. However, it is specifically stated that a citation of a published resource herein shall not be construed as an admission that such is prior art to the present invention. Also, in the event that one or more of the incorporated published resources differs from or contradicts this application, including but not limited to defined terms, term usage, described techniques, or the like, this application controls.

While various embodiments of the present invention have been shown and described herein, it is emphasized that such embodiments are provided by way of example only. Numerous variations, changes and substitutions may be made without departing from the invention herein in its various embodiments. Specifically, and for whatever reason, for any grouping of compounds, nucleic acid sequences, polypeptides including specific proteins including functional enzymes, metabolic pathway enzymes or intermediates, elements, or other compositions, or concentrations stated or otherwise presented herein in a list, table, or other grouping (such as metabolic pathway enzymes shown in a figure), unless clearly stated otherwise, it is intended that each such grouping provides the basis for and serves to identify various subset embodiments, the subset embodiments in their broadest scope comprising every subset of such grouping by exclusion of one or more members (or subsets) of the respective stated grouping. Moreover, when any range is described herein, unless clearly stated otherwise, that range includes all values therein and all sub-ranges therein. Accordingly, it is intended that the invention be limited only by the spirit and scope of appended claims, and of later claims, and of either such claims as they may be amended during prosecution of this or a later application claiming priority hereto.

### EXAMPLES

Unless otherwise indicated, the following are examples planned to be conducted or actually conducted in Boulder, Colo., USA. Unless indicated otherwise, temperature is in degrees Celsius and pressure is at or near atmospheric pressure at approximately 5340 feet (1628 meters) above sea level. It is noted that work done at external analytical and synthetic facilities is not conducted at or near atmospheric pressure at approximately 5340 feet (1628 meters) above sea level. All reagents, unless otherwise indicated, are obtained commercially. Species and other phylogenic identifications are according to the classification known to a person skilled in the art of microbiology.

The meaning of abbreviations is as follows: “C” means Celsius or degrees Celsius, as is clear from its usage, “s” means second(s), “min” means minute(s), “h,” “hr,” or “hrs” means hour(s), “psi” means pounds per square inch, “nm” means nanometers, “d” means day(s), “μL” or “uL” or “ul” means microliter(s), “mL” means milliliter(s), “L” means liter(s), “mm” means millimeter(s), “nm” means nanometers, “mM” means millimolar, “μM” or “uM” means micromolar, “M” means molar, “mmol” means millimole(s), “μmol” or “uMol” means micromole(s)”, “g” means gram(s), “μg” or “ug” means microgram(s) and “ng” means nanogram(s), “PCR” means polymerase chain reaction, “OD” means optical density, “OD₆₀₀” means the optical density measured at a photon wavelength of 600 nm, “kDa” means kilodaltons, “g” means the gravitation constant, “bp” means base pair(s), “kbp” means kilobase pair(s), “% w/v” means weight/volume percent, “% v/v” means volume/volume percent, “IPTG” means isopropyl-μ-D-thiogalactopyranoiside, “RBS” means ribosome binding site, “rpm” means revolutions per minute, “HPLC” means high performance liquid chromatography, and “GC” means gas chromatography.

### Example 1

### Enzyme Evolution to Evolve an Enzyme Having Fatty Acid O-Methyltransferase Activity (Prophetic)

The following steps and methods are related to evolving an enzyme from mutants of an enzyme having a similar catalytic activity to Juvenile hormone (JH) acid O-methyltransferase (JHAMT).

Part I: Construct mutant libraries of the Juvenile hormone (JH) acid O-methyltransferase (JHAMT) gene, DmJHAMT. This is done using methods known to those skilled in the art, such as described in U.S. Patent Publication US2006/0084098, published Apr. 20, 2006, incorporated by reference for this teaching; and also from Sambrook and Russell, 2001.

More particularly, a mutant library of the DmJHAMT gene that will constructed for use for screening. A polynucleotide exhibiting enzymatic activity of the DmJHAMT gene from Drosophila melanogaster will be cloned into an appropriate expression system for E. coli. Cloning of a codon and expression of the optimized DmJHAMT gene will be accomplished via gene synthesis supplied from a commercial supplier using standard techniques. This will bypass the need for manipulating Drosophila melanogaster. The gene will be synthesized with an eight amino acid N- or C-terminal tag to enable affinity based protein purification. Once obtained using standard methodology, the gene will be cloned into an expression system using standard techniques.

The plasmid containing the above-described DmJHAMT gene will be mutated by standard methods resulting in a large library of DmJHAMT mutants (>10⁶). The mutant DmJHAMT sequences will be excised from these plasmids and again cloned into an expression vector, generating a final library of greater than 10⁶ clones for subsequent screening. These numbers ensure a greater than 99% probability that the library will contain a mutation in every amino acid encoded by the DmJHAMT sequence. It is acknowledged that each method of creating a mutational library has its own biases, including transformation into mutator strains of E. coli, error prone PCR, and in addition more site directed mutagenesis.

One possible method is the use of the XL1-Red mutator strain, which is deficient in several repair mechanisms necessary for accurate DNA replication and generates mutations in plasmids at a rate 5,000 times that of the wild-type mutation rate, which may be employed using appropriate materials following a manufacturer's instructions (See Stratagene XL-1 Red competent cells, Stratagene, La Jolla, Calif. USA). This technique or other techniques known to those skilled in the art may be employed and then a population of such mutants, e.g., in a library, is evaluated, such as by a screening or selection method, to identify clones having a suitable or favorable mutation.

Part II: Screen a mutant library of DmJHAMT or other sources for increased fatty acid O-methyltransferase activity. With the successful construction of a mutant DmJHAMT library, it will be possible to screen this library for increased fatty acid O-methyltransferase activity. The screening process will be designed to screen the entire library of greater than 10⁶ mutants.

A routine screening approach will be employed using standard methods known in the art to isolate affinity tagged enzymes as well for the detection of the fatty acid O-methyltransferase products. Clones will be pooled and enzymes purified. Subsequently, purified enzyme will be screened in well glass plates with solubilized palmitic acid, farnesoic acid or lauric acid and S-adenosylmethionine.

Screening every member of a library of greater than 10⁶ mutants is time-consuming. An alternative pooling method may be used. This approach groups several tens, hundreds, or thousand mutants in a collection or pool. Standard methods will be used to replicate each of these mutant pools and screen them in multi-well plate format. This grouping will be performed in such a way as to enable the future separation of members of this pool. If a member of a particular pool contains the desired increased fatty acid O-methyltransferase activity the pool will be subdivided into smaller groups until the individual clone(s) containing the desired enzyme is isolated. It is expected that any screening assay will need to be evaluated and optimized, possibly in an iterative fashion.

The fatty acid O-methyl transferase activity of DmJHAMT and DmJHAMT mutants may be measured continuously by detection of reaction products. Specifically, the S-adenosylhomocysteine product may be converted by S-adenosylhomocysteine hydrolase into adenosine and homocysteine. Common spectrophotometry methods may be used to detect and quantify these products. Fatty acid O-methyl transferase activity may be measured in vivo or in vitro using these methods.

In vitro example: Fatty acid O-methyl transferase activity of cell lysates may be measured in multi well plates by detecting the decrease in absorbance at 265 nm upon conversion of S-adenosylmethionine into homocysteine and inosine by a 3 step, in situ process that requires fatty acid O-methyl transferase, S-adenosylhomocysteine hydrolase and adenosine deaminase activities.

In vivo example: Fatty acid O-methyl transferase activity of whole cells and cell libraries may be measured in multi well plates by detecting the increase in fluorescence upon conversion of S-adenosylmethionine into homocysteine by a in vivo 2 step process that requires fatty acid O-methyl transferase and S-adenosylhomocysteine hydrolase activities. The homocysteine may leave the cell and be quantified with a thiol detecting fluorescent dye (example: CPM). This process may also be used in a high-throughput device to measure and sort cells encased within water/oil/water emulsions having fatty acid O-methyl transferase activity, for example by a Fluorescence Activated Cell Sorter (FACS). Use of a FACS to sort cells encased within water/oil/water emulsions has been described by Aharoni et al., Chem. Biol. 12:1281 (2005).

As a result of such efforts a functional variant so obtained demonstrates activity for forming a methyl ester of a fatty acid that is completely or largely saturated that is at least 10, 20, 30, 40, 50, 60 70, 80, 90, 100, or greater than 150 or 200 percent greater than the activity for such reaction by unmodified DmJHAMT.

Other genes and proteins they encode may be used for development of a suitably functional (having desired activity and specificity) O-methyltransferase for conversion of a fatty acid to a FAME or other product. Among these are those listed in Table 3, incorporated into this Example. The same approach as described above in this example is applied to one or more of these to obtain a suitable O-methyltransferase, including having an activity and selectivity suitable for commercial production activities, etc.

### Example 2

### General Example of Genetic Modification to a Host Cell (Prophetic and Non-Specific)

This example is meant to describe a non-limiting approach to genetic modification of a selected microorganism to introduce a nucleic acid sequence of interest. Alternatives and variations are provided within this general example. The methods of this example are conducted to achieve a combination of desired genetic modifications in a selected microorganism species, such as a combination of genetic modifications selected from those shown in FIGS. 1 and/or 2, and their equivalents.

A gene or other nucleic acid sequence segment of interest is identified in a particular species (such as E. coli as described above) and a nucleic acid sequence comprising that gene or segment is obtained. For clarity below the use of the term “segment of interest” below is meant to include both a gene and any other nucleic acid sequence segment of interest. One example of a method used to obtain a segment of interest is to acquire a culture of a microorganism, where that microorganism's genome includes the gene or nucleic acid sequence segment of interest.

Based on the nucleic acid sequences at the ends of or adjacent the ends of the segment of interest, 5′ and 3′ nucleic acid primers are prepared. Each primer is designed to have a sufficient overlap section that hybridizes with such ends or adjacent regions. Such primers may include enzyme recognition sites for restriction digest of transposase insertion that could be used for subsequent vector incorporation or genomic insertion. These sites are typically designed to be outward of the hybridizing overlap sections. Numerous contract services are known that prepare primer sequences to order (e.g., Integrated DNA Technologies, Coralville, Iowa USA).

Once primers are designed and prepared, polymerase chain reaction (PCR) is conducted to specifically amplify the desired segment of interest. This method results in multiple copies of the region of interest separated from the microorganism's genome. The microorganism's DNA, the primers, and a thermophilic polymerase are combined in a buffer solution with potassium and divalent cations (e.g., Mg or Mn) and with sufficient quantities of deoxynucleoside triphosphate molecules. This mixture is exposed to a standard regimen of temperature increases and decreases. However, temperatures, components, concentrations, and cycle times may vary according to the reaction according to length of the sequence to be copied, annealing temperature approximations and other factors known or readily learned through routine experimentation by one skilled in the art.

In an alternative embodiment the segment of interest may be synthesized, such as by a commercial vendor, and prepared via PCR, rather than obtaining from a microorganism or other natural source of DNA.

The nucleic acid sequences then are purified and separated, such as on an agarose gel via electrophoresis. Optionally, once the region is purified it can be validated by standard DNA sequencing methodology and may be introduced into a vector. Any of a number of vectors may be used, which generally comprise markers known to those skilled in the art, and standard methodologies are routinely employed for such introduction. Commonly used vector systems are pSMART (Lucigen, Middleton, Wis.), pET E. COLi EXPRESSION SYSTEM (Stratagene, La Jolla, Calif.), pSC-B StrataClone Vector (Stratagene, La Jolla, Calif.), pRANGER-BTB vectors (Lucigen, Middleton, Wis.), and TOPO vector (Invitrogen Corp, Carlsbad, Calif., USA). Similarly, the vector then is introduced into any of a number of host cells. Commonly used host cells are E. cloni 10G (Lucigen, Middleton, Wis.), E. cloni 10GF′ (Lucigen, Middleton, Wis.), StrataClone Competent cells (Stratagene, La Jolla, Calif.), E. coli BL21, E. coli BW25113, and E. coli K12 MG1655. Some of these vectors possess promoters, such as inducible promoters, adjacent the region into which the sequence of interest is inserted (such as into a multiple cloning site), while other vectors, such as pSMART vectors (Lucigen, Middleton, Wis.), are provided without promoters and with dephosphorylated blunt ends. The culturing of such plasmid-laden cells permits plasmid replication and thus replication of the segment of interest, which often corresponds to expression of the segment of interest.

Various vector systems comprise a selectable marker, such as an expressible gene encoding a protein needed for growth or survival under defined conditions. Common selectable markers contained on backbone vector sequences include genes that encode for one or more proteins required for antibiotic resistance as well as genes required to complement auxotrophic deficiencies or supply critical nutrients not present or available in a particular culture media. Vectors also comprise a replication system suitable for a host cell of interest.

The plasmids containing the segment of interest can then be isolated by routine methods and are available for introduction into other microorganism host cells of interest. Various methods of introduction are known in the art and can include vector introduction or genomic integration. In various alternative embodiments the DNA segment of interest may be separated from other plasmid DNA if the former will be introduced into a host cell of interest by means other than such plasmid.

While steps of the above general prophetic example involve use of plasmids, other vectors known in the art may be used instead. These include cosmids, viruses (e.g., bacteriophage, animal viruses, plant viruses), and artificial chromosomes (e.g., yeast artificial chromosomes (YAC) and bacteria artificial chromosomes (BAC)).

Host cells into which the segment of interest is introduced may be evaluated for performance as to a particular enzymatic step, and/or tolerance or bio-production of a chemical compound of interest. Selections of better performing genetically modified host cells may be made, selecting for overall performance, tolerance, or production or accumulation of the chemical of interest.

It is noted that this procedure may incorporate a nucleic acid sequence for a single gene (or other nucleic acid sequence segment of interest), or multiple genes (under control of separate promoters or a single promoter), and the procedure may be repeated to create the desired heterologous nucleic acid sequences in expression vectors, which are then supplied to a selected microorganism so as to have, for example, a desired complement of enzymatic conversion step functionality for any of the herein-disclosed metabolic pathways. However, it is noted that although many approaches rely on expression via transcription of all or part of the sequence of interest, and then translation of the transcribed mRNA to yield a polypeptide such as an enzyme, certain sequences of interest may exert an effect by means other than such expression.

The specific laboratory methods used for the above approaches are well-known in the art and may be found in various references known to those skilled in the art, such as Sambrook and Russell, Molecular Cloning: A Laboratory Manual, Third Edition 2001 (volumes 1-3), Cold Spring Harbor Laboratory Press, Cold Spring Harbor, N.Y. (hereinafter, Sambrook and Russell, 2001). These methods include codon-optimization of sequences for introduction of a non-native nucleic acid sequence into a selected microorganism.

As an alternative to the above, other genetic modifications may also be practiced, such as a deletion of a nucleic acid sequence of the host cell's genome. One non-limiting method to achieve this is by use of Red/ET recombination, known to those of ordinary skill in the art and described in U.S. Pat. Nos. 6,355,412 and 6,509,156, issued to Stewart et al. and incorporated by reference herein for its teachings of this method. Material and kits for such method are available from Gene Bridges (Gene Bridges GmbH, Dresden, Germany, www.genebridges.com), and the method may proceed by following the manufacturer's instructions. Targeted deletion of genomic DNA may be practiced to alter a host cell's metabolism so as to reduce or eliminate production of undesired metabolic products. This may be used in combination with other genetic modifications such as described above in this general example. In this detailed description, reference has been made to multiple embodiments and to the accompanying drawings in which is shown by way of illustration specific exemplary embodiments in which the invention may be practiced. These embodiments are described in sufficient detail to enable those skilled in the art to practice the invention, and it is to be understood that modifications to the various disclosed embodiments may be made by a skilled artisan.

### Example 3

### Modifying Microorganisms to Improve Use of Carbon Monoxide and/or Carbon Dioxide as Carbon Source(s) for Bio-Fermentations (Prophetic)

This example describes developing microorganisms that have improved utilization, including improved rates, titers and yields (conversion efficiencies) for production of a chemical product, where that microorganism has or is provided with the capacity to utilize carbon monoxide and/or carbon dioxide as carbon source(s) and also the capacity to utilize hydrogen as a source to generate reducing equivalents (e.g., NADH, NADPH). These metabolic capacities may be native to the microorganism, native and improved, or introduced such as by teachings presented herein or elsewhere known in the art.

To implement such improved utilization, the microorganism is modified so as to provide or increase proteins that catalyze one or more of the enzymatic conversions numbered 1 through 9, inclusive, in FIG. 1, further described and exemplified in Table 1 (step numbers 1-9). That is, the microorganism is modified to provide or increase the enzymatic reactions that include a portion of a myo-inositol pathway.

Using methods known in the art and/or described herein, such as in Example 2, genes encoding proteins having the desired enzymatic activities for such conversions are introduced into the microorganism. These may be provided in plasmids comprising expression vectors of such genes, or may be introduced into the microorganism genome. When starting with the sequences provided in Table 1 (i.e., SEQ ID NOs:001, 003, 005, 007, 009, 011, 013, 015, 017), these sequences may be codon-optimized for the microorganism being modified, such as by any of the algorithms known to and use by those skilled in the art. One non-limiting example is the codon-optimization software provided by a commercial DNA sequence provider, DNA2.0 (Menlo Park, Calif.).

Alternatively, native gene(s) in a selected microorganism may be overexpressed, underexpressed, or otherwise modified such as to improve specificity and/or rate.

In one particular example, the selected microorganism is Cupriavidus necator, a species known to utilize hydrogen and carbon dioxide to produce more complex organic compounds for its growth and maintenance. Into a Cupriavidus necator strain, such as DSM542, each of SEQ ID NOs:001, 003, 005, 007, 009, 011, 013, 015, 017 are first codon-optimized such as by using the available codon-optimizing software, such as from DNA2.0 (Menlo Park, Calif.), and produced by a gene synthesis service provider, such as DNA2.0. The codon-optimized nucleic acid sequences so provided are introduced to the microorganism cells by methods known to those skilled in the art. In one particular embodiment, these sequences are combined together into plasmid DNA of reasonable size to reduce the number of different introduced plasmids. In a second particular embodiment, these sequences are introduced into the strain genome, such as by the methods described in Example 2 above, under the control of suitable promoters.

Expression of these genes is evaluated and improved so as to obtain a modified microorganism having the capability to convert fructose-6-phosphate to dihydroxyacetone phosphate and malonate semialdehyde. In various embodiments the percentage of carbon source converted to malonate semialdehyde is greater than 25%.

Another approach is to utilize native nucleic acid sequences by overexpression and/or modification to obtain one or more, including all, of the enzymatic activities of steps 1 through 9, inclusive, in a selected microorganism. For example, considering a Cupriavidus necator strain, such as DSM542, certain sequences in this strain are known to have relative high homologies to the proteins of steps 1 to 9, and particularly to SEQ ID NOs:002, 004, 006, 008, 010, 012, 014, 016, and 018.

In other embodiments, the above-described modifications are made to microorganism cells of Oligotropha carboxidovorans to provide the capacity to convert carbon dioxide to malonate semialdehyde.

Also, modifications may similarly be made with regard to steps 10 and 11 of FIG. 1 and Table 1, to provide or increase conversion capability in a microorganism cell from malonate semialdehyde to malonate to malonate-CoA.

FIG. 2 summarizes homologues of most of the proteins of Table 1, steps 1 to 11, in the species C. necator and O. carboxidovorans. These homolog sequences are candidates for use and/or further modification so as to obtain a desired enzymatic conversion indicated in FIG. 1 and Table 1 for the indicated steps. Modifications to achieve a suitable activity and a suitable specificity may be made such as by approaches described herein.

Any of the microorganisms of this example may be further modified, such as described in the examples below, so as to have additional metabolic capability and improved conversion efficiency.

### Example 4

### Additional Modifications of Microorganisms to Improve Bio-fermentations (Prophetic)

A microorganism such as described in Example 3 is further modified to provide or increase enzymatic functions so as to convert malonate semialdehyde to one or more organic chemicals including a fatty acid.

The starting microorganism cell is provided with nucleic acid molecules that provide and/or increase the enzymatic conversions indicated in steps 10 through 13 to achieve a desired rate of such conversion. It is noted that ‘step 12’ actually comprises multiple steps in fatty acid synthesis. In exemplary embodiments, this results in expression of many, if not all, of the following proteins and their corresponding enzymatic activities (exemplary specific proteins and step number provided):

Aldehyde dehydrogenase (Ald, 10)

Malonyl-CoA synthetase (MatB, 11)

Malonyl-CoA-[acyl carrier protein (“ACP”)] transacylase (FabD, 12)

β-ketoacyl-ACP synthase (FabB, FabF, FabH, 12)

β-ketoacyl-ACP reductase (FabG, 12)

β-hydroxyacyl-ACP dehydratase (FabA, FabZ, 12)

Enoyl-ACP reductase (FabI, FabK, 12)

Acyl-ACP thioesterase (TesA, 13)

When adding the genes required for any of these activities, the nucleic acid sequences may be obtained by finding homologous sequences in a particular microorganism, including in the genome of the starting microorganism. Codon-optimizing, including from sequences provided herein, may be conducted when making a sequence that is derived from a different microorganism. Evaluations are conducted, and genetic modifications are made as needed, to achieve a desired specificity and activity for the particular enzymatic reaction.

Considering the microorganism Cupriavidus necator, such as strain DSM542, a BlastP comparison with some of the above genes provides lists of candidate native nucleic acid sequences that could be used, or modified, to achieve a desired increase in one of the enzymatic conversion steps 9 through 13.

For example, based on the—E. coli FabD malonyl-CoA-[ACP] transacylase (SEQ ID NO:023) as the query sequence, the following sequence is identified as a candidate sequence in and for Cupriavidus necator: gi|113868530|ref|YP_{—}727019.1|.

Similarly, for the FabH β-ketoacyl-ACP synthase the following sequence is identified: gi|113868531|ref|YP_{—}727020.1|. Two candidates for FabF β-ketoacyl-ACP synthase were identified from a BlastP with SEQ ID NO:029 as the query sequence: gi|113868527|ref|YP_{—}727016.1| and gi|116695606|ref|YP_{—}841182.1|. These same two candidates were identified when the query sequence was the E. coli FabB β-ketoacyl-ACP synthase (SEQ ID NO:030).

The following table provides identifiers for sequences in Cupriavidus necator that are found homologous to the E. coli FabG β-ketoacyl-ACP reductase (3-oxoacyl-ACP-reductase) (SEQ ID NO:025):

TABLE 4

| O. carboxidovorans | O. carboxidovorans | O. carboxidovorans |
|---|---|---|
| Additional Candidate NCBI | Additional Candidate NCBI | Additional Candidate NCBI |
| Reference Sequence | Reference Sequence | Reference Sequence |
| (version) | (version) | (version) |
| gi\|113868529\|ref\|YP_727018.1\| | gi\|113868428\|ref\|YP_726917.1\| | gi\|116695451\|ref\|YP_841027.1\| |
| gi\|113867453\|ref\|YP_725942.1\| | gi\|113867344\|ref\|YP_725833.1\| | gi\|116695846\|ref\|YP_841422.1\| |
| gi\|113867981\|ref\|YP_726470.1\| | gi\|116695241\|ref\|YP_840817.1\| | gi\|116694614\|ref\|YP_728825.1\| |
| gi\|113868147\|ref\|YP_726636.1\| | gi\|116695722\|ref\|YP_841298.1\| | gi\|116696432\|ref\|YP_842008.1\| |
| gi\|116695840\|ref\|YP_841416.1\| | gi\|116694061\|ref\|YP_728272.1\| | gi\|113867868\|ref\|YP_726357.1\| |
| gi\|113869118\|ref\|YP_727607.1\| | gi\|113866956\|ref\|YP_725445.1\| | gi\|113866900\|ref\|YP_725389.1\| |
| gi\|116696446\|ref\|YP_842022.1\| | gi\|113868440\|ref\|YP_726929.1\| | gi\|113869529\|ref\|YP_728018.1\| |
| gi\|116694315\|ref\|YP_728526.1\| | gi\|116694682\|ref\|YP_728893.1\| | gi\|116695085\|ref\|YP_840661.1\| |
| gi\|116694338\|ref\|YP_728549.1\| | gi\|113866770\|ref\|YP_725259.1\| | gi\|116695734\|ref\|YP_841310.1\| |
| gi\|113867286\|ref\|YP_725775.1\| | gi\|116694156\|ref\|YP_728367.1\| | gi\|113866629\|ref\|YP_725118.1\| |
| gi\|116695278\|ref\|YP_840854.1\| | gi\|113867502\|ref\|YP_725991.1\| | gi\|116695726\|ref\|YP_841302.1\| |
| gi\|113867797\|ref\|YP_726286.1\| | gi\|113866875\|ref\|YP_725364.1\| | gi\|116694599\|ref\|YP_728810.1\| |
| gi\|116695770\|ref\|YP_841346.1\| | gi\|116694685\|ref\|YP_728896.1\| | gi\|116694585\|ref\|YP_728796.1\| |
| gi\|116694022\|ref\|YP_728233.1\| | gi\|116695830\|ref\|YP_841406.1\| | gi\|116694581\|ref\|YP_728792.1\| |
| gi\|116695384\|ref\|YP_840960.1\| | gi\|116694347\|ref\|YP_728558.1\| | gi\|116695741\|ref\|YP_841317.1\| |
| gi\|113867306\|ref\|YP_725795.1\| | gi\|116695926\|ref\|YP_841502.1\| | gi\|116694341\|ref\|YP_728552.1\| |
| gi\|116695635\|ref\|YP_841211.1\| | gi\|116694593\|ref\|YP_728804.1\| | gi\|113866193\|ref\|YP_724682.1\| |
| gi\|113867542\|ref\|YP_726031.1\| | gi\|116694550\|ref\|YP_728761.1\| | gi\|116695568\|ref\|YP_841144.1\| |
| gi\|113867353\|ref\|YP_725842.1\| | gi\|113869434\|ref\|YP_727923.1\| | gi\|116695287\|ref\|YP_840863.1\| |
| gi\|116695184\|ref\|YP_840760.1\| | gi\|116694600\|ref\|YP_728811.1\| | gi\|113869676\|ref\|YP_728165.1\| |
| gi\|116694602\|ref\|YP_728813.1\| | gi\|113866064\|ref\|YP_724553.1\| | gi\|113867547\|ref\|YP_726036.1\| |
| gi\|113868128\|ref\|YP_726617.1\| | gi\|116694638\|ref\|YP_728849.1\| | gi\|113866289\|ref\|YP_724778.1\| |
| gi\|116695020\|ref\|YP_840596.1\| | gi\|116694259\|ref\|YP_728470.1\| | gi\|113867750\|ref\|YP_726239.1\| |
| gi\|116695668\|ref\|YP_841244.1\| | gi\|116694664\|ref\|YP_728875.1\| | gi\|116696287\|ref\|YP_841863.1\| |
| gi\|116694617\|ref\|YP_728828.1\| | gi\|113867940\|ref\|YP_726429.1\| | gi\|116694597\|ref\|YP_728808.1\| |
| gi\|116694552\|ref\|YP_728763.1\| | gi\|116695703\|ref\|YP_841279.1\| | gi\|116694624\|ref\|YP_728835.1\| |
| gi\|116696275\|ref\|YP_841851.1\| | gi\|116695910\|ref\|YP_841486.1\| | gi\|113867188\|ref\|YP_725677.1\| |

BlastP homology results for the Carboxydothermus hydrogenoformans FabZ (SEQ ID NO:026) against C. necator provided two candidates in the latter: gi|113868023|ref|YP_{—}726512.1| and gi|116695601|ref|YP_{—}841177.1|.

The following table provides BlastP homology results for the E. coli FabI (SEQ ID NO:027) enoyl-ACP reductase against C. necator:

TABLE 5

| C. necator Additional | C. necator Additional |
|---|---|
| Candidate NCBI Reference | Candidate NCBI Reference |
| Sequence (version) | Sequence (version) |
| gi\|113868381\|ref\|YP_726870.1\| | gi\|113867286\|ref\|YP_725775.1\| |
| gi\|38637922\|ref\|NP_942896.1\| | gi\|113867502\|ref\|YP_725991.1\| |
| gi\|116695568\|ref\|YP_841144.1\| | gi\|116694550\|ref\|YP_728761.1\| |
| gi\|113866900\|ref\|YP_725389.1\| | gi\|116694682\|ref\|YP_728893.1\| |
| gi\|113869529\|ref\|YP_728018.1\| | gi\|113866875\|ref\|YP_725364.1\| |
| gi\|116694061\|ref\|YP_728272.1\| | gi\|116695830\|ref\|YP_841406.1\| |
| gi\|113866064\|ref\|YP_724553.1\| | gi\|113868440\|ref\|YP_726929.1\| |
| gi\|116694602\|ref\|YP_728813.1\| | gi\|113869118\|ref\|YP_727607.1\| |
| gi\|116695241\|ref\|YP_840817.1\| | gi\|116694156\|ref\|YP_728367.1\| |
| gi\|116695184\|ref\|YP_840760.1\| | gi\|116694599\|ref\|YP_728810.1\| |
| gi\|113869434\|ref\|YP_727923.1\| | gi\|116694624\|ref\|YP_728835.1\| |
| gi\|116696275\|ref\|YP_841851.1\| | gi\|113868529\|ref\|YP_727018.1\| |
| gi\|116695085\|ref\|YP_840661.1\| | gi\|113867542\|ref\|YP_726031.1\| |
| gi\|116694581\|ref\|YP_728792.1\| | gi\|116694022\|ref\|YP_728233.1\| |
| gi\|116695770\|ref\|YP_841346.1\| | |

Similarly, the following table provides BlastP homology results for the E. coli FabK (SEQ ID NO:028) enoyl-ACP reductase against C. necator:

TABLE 6
C. necator Additional Candidate
NCBI Reference Sequence (version)

gi|116694708|ref|YP_728919.1|
gi|116694318|ref|YP_728529.1|
gi|116694354|ref|YP_728565.1|
gi|116694609|ref|YP_728820.1|
gi|116695699|ref|YP_841275.1|
gi|38638056|ref|NP_943030.1|
gi|116696267|ref|YP_841843.1|
gi|113866323|ref|YP_724812.1|
gi|38637919|ref|NP_942893.1|
gi|116694178|ref|YP_728389.1|

The BlastP of E. coli acyl-ACP-thioesterase (SEQ ID NO:032) against C. necator yielded the following sequence in the latter: gi|113867511|ref|YP_{—}726000.1|.

Where more than one gene provides the same function, a particular microorganism may comprise modifications of one or more of these.

Accordingly, combinations of genetic modifications are made to a selected microorganism, such as C. necator strain DSM 542, to provide a desired enzymatic pathway connecting from the Calvin Benson Cycle through a fatty acid synthase pathway to a fatty acid. Relevant modifications are made to decrease or eliminate enzymatic activity of certain proteins to reduce diversion of carbon and energy to other pathways, intermediates and end products (e.g., see Example 6). Combinations of modifications result in increased efficiency of conversion of carbon dioxide and/or carbon monoxide to a desired fatty acid-based product.

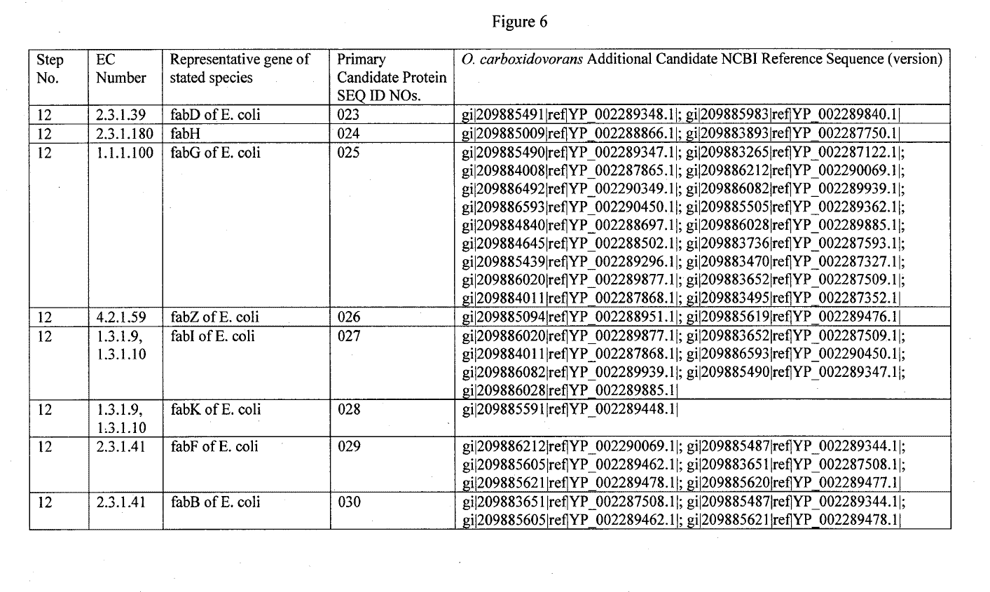

In other embodiments, the above-described types of modifications are made to microorganism cells of Oligotropha carboxidovorans, such as by using and/or modifying candidate sequences showing a level of homology. FIG. 6 summarizes proteins in that species that demonstrate a homology to proteins of enzymatic conversion steps described in this example.

In addition to specific products described elsewhere herein (e.g., see Examples 1 and 5), a fatty acid produced in this example may be converted to other fatty acid derivatives, such as described in U.S. Patent Application No. 2010/0154293, published Jun. 24, 2010, and incorporated by reference for its teachings of how to make fatty acid derivative products, and those products. Among such fatty acid derivatives are esters of fatty acids, such as methyl, ethyl, butyl and longer chain alkyl additions.

### Example 5

### Construction of C. necator Strains for Evaluation (Prophetic)

Part 1: Gene Deletions

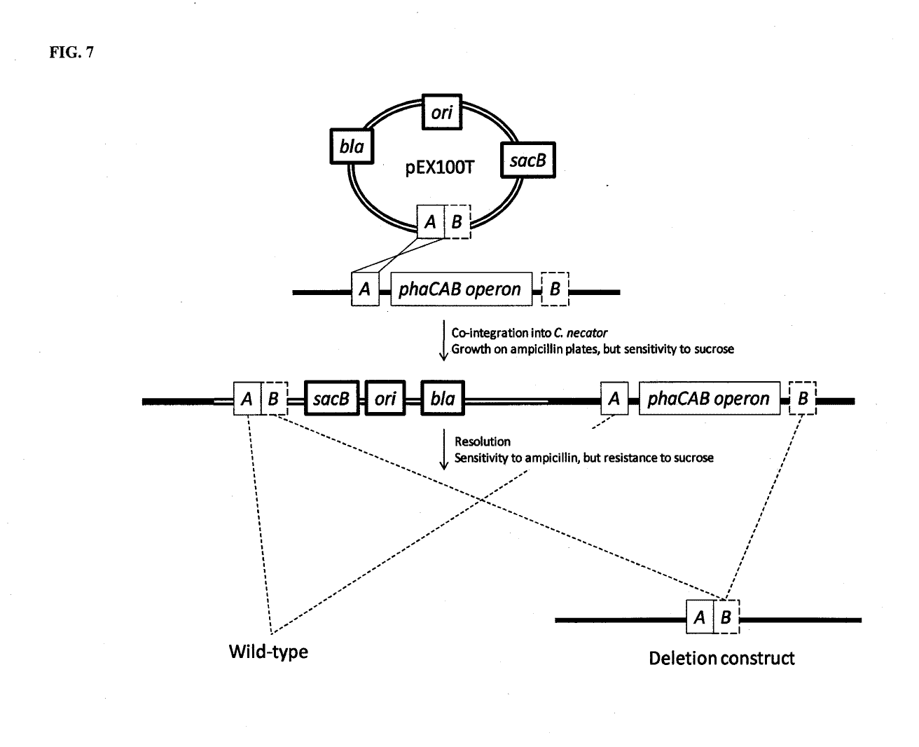

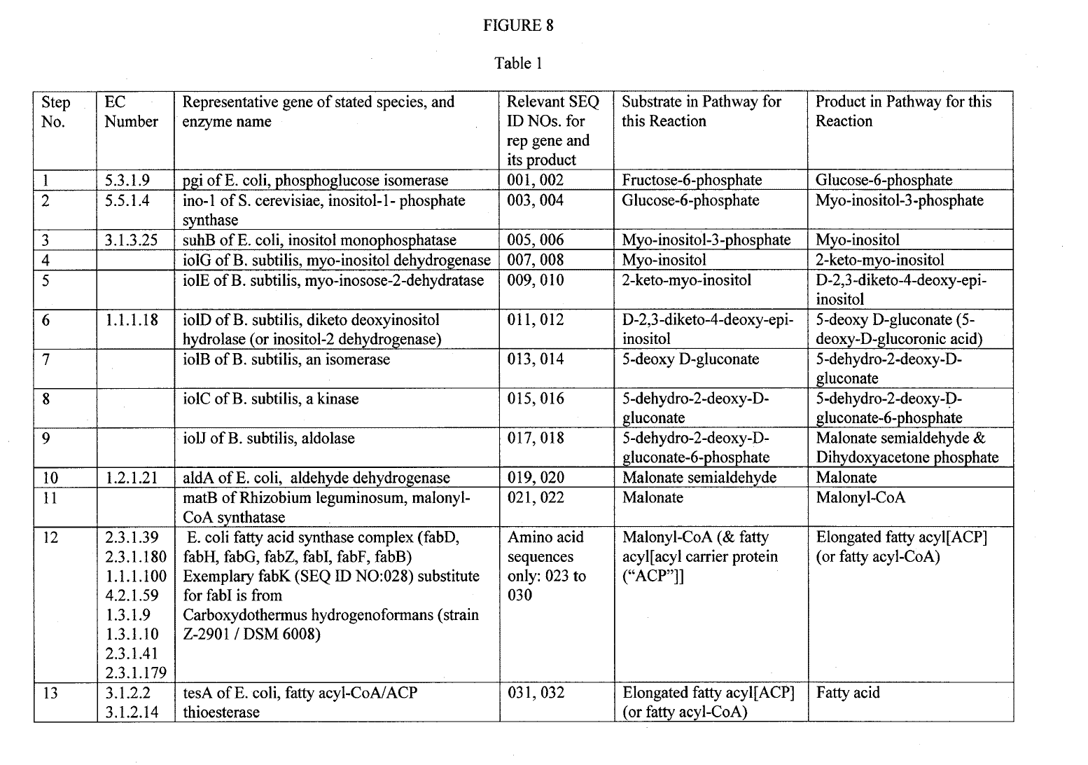

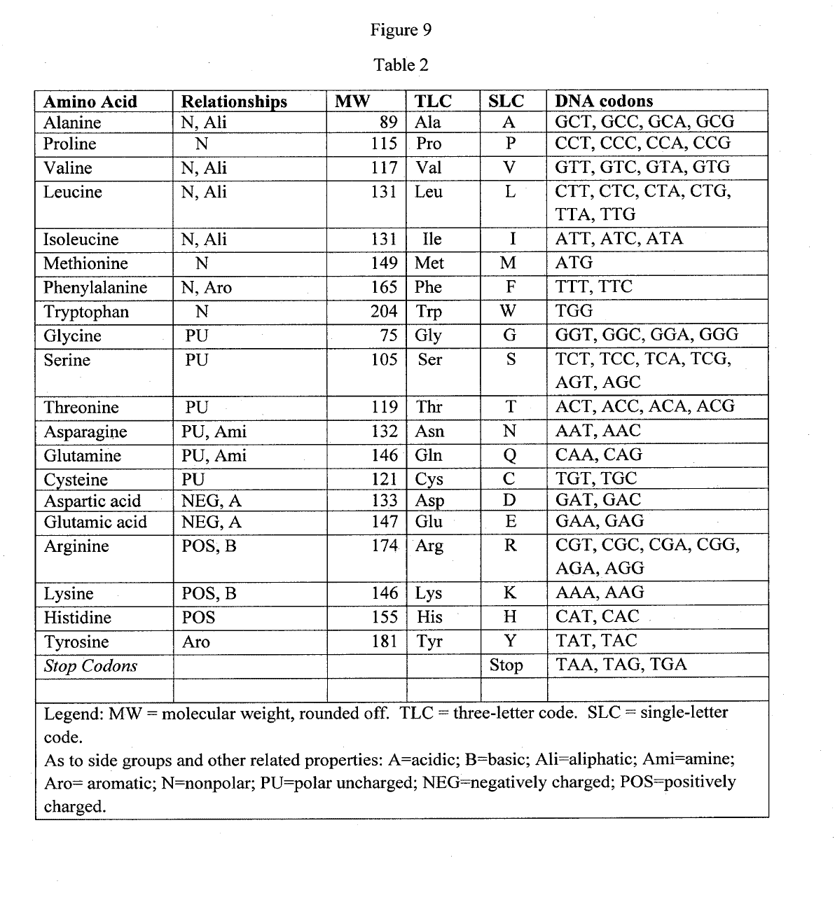

The homologous recombination method using integration of counterselectable suicide vectors, is employed for gene deletion in C. necator strains. This method is known to those of ordinary skill in the art. The method integrates a target sequence including both a selectable marker and counterselectable marker via homologous recombination performed by host recombination machinery. Integrants are selected via the selectable marker, following the approach depicted in FIG. 7. The markers are then removed by counterselection and 2 genotypes are distinguished by screening via PCR, one would be wild type, the second the desired gene deletion, integration or replacement.

Specific gene deletions in C. necator are constructed by creating counterselectable suicide vectors that will delete the genes or operons. These vectors are constructed by gene synthesis or via cloning using overlapping PCR.

Table 7 below list the desired genes and or operons that are deleted singly and in combination in C. necator strains that produce free fatty acids and fatty acid derived products including FAMEs.

TABLE 7

| Gene/Operon Name | Function |
|---|---|
| H16_A0459 through H16_A0464 | Fatty acid Beta oxidation |
| H16_A1526 through H6_A1531 | Fatty acid Beta oxidation |
| phaCAB | Polyhydroxybutyrate |
| | formation |
| sdaA | Serine deaminase |
| tdcB | Threonine/serine deaminase |
| h16_B0620 | Serine deaminase |

Part 2: Construction of Plasmids for Gene Overexpression

In addition to the construction of gene deletions and integrations in C. necator, replicating plasmids may be used to introduce genetic modifications into C. necator strains including those that enable the overexpression of desired genes and the increase in desired enzyme functions. Cloning and expression of genes can be performed in numerous plasmids. For example small broad host range vectors may be used for expression such as pBT-3 (see U.S. Patent Publication No. 2007/0059768, published Mar. 15, 2007, and incorporated by reference for its teachings of the construction and use of these vectors.) In addition to overexpressing the genes and enzymes listed in Table 1 on plasmids enabling the production of free fatty acid in C. necator, the production of FAMEs requires the expression of a Fatty acid O-methyl transferase. As discussed above in Example 1, several different sequences may be expressed having fatty-O-methyltransferase activity. Expression of these genes or improved mutants or homologous alternative thereof may be expressed in C. necator on plasmids. In addition any gene listed in Table 1 or fatty acid O-methyltransferases is integrated into the chromosome(s) of C. necator using standard methods.

Part 2: Construction of Strains

Any combination of gene deletions and gene overexpressions described above may be incorportated into a single C. necator strain for the production of free fatty acids and or FAMEs.

### Example 6

### Production of FAME and/or Free Fatty Acid (Prophetic)

An inoculum of a genetically modified microorganism that possesses a free fatty acid or FAME production pathway and other genetic modifications as described above is provided to a culture vessel to which also is provided a liquid media comprising nutrients at concentrations sufficient for a desired bio-process culture period.

The final broth (comprising microorganism cells, largely ‘spent’ media product, the latter at concentrations, in various embodiments, at least 1, 2, 5, 10, 30, 50, 75 or 100 grams/liter) is collected and subjected to separation and purification steps so that the FAME or free fatty acid is obtained in a relatively purified state. Separation and purification steps may proceed by any of a number of approaches combining various methodologies, which may include centrifugation, concentration, filtration, reduced pressure evaporation, liquid/liquid phase separation Principles and details of standard separation and purification steps are known in the art, for example in “Bioseparations Science and Engineering,” Roger G. Harrison et al., Oxford University Press (2003), and Membrane Separations in the Recovery of Biofuels and Biochemicals—An Update Review, Stephen A. Leeper, pp. 99-194, in Separation and Purification Technology, Norman N. Li and Joseph M. Calo, Eds., Marcel Dekker (1992), incorporated herein for such teachings. The particular combination of methodologies is selected from those described herein, and in part is based on the concentration of free fatty acid and/or FAME and other components in the final broth.

Where methods and steps described above indicate certain events occurring in certain order, those of ordinary skill in the art will recognize that the ordering of certain steps may be modified and that such modifications are in accordance with the variations of the invention. Additionally, certain steps may be performed concurrently in a parallel process when possible, as well as performed sequentially.

The embodiments, variations, sequences, and figures described herein should provide an indication of the utility and versatility of the present invention. Other embodiments that do not provide all of the features and advantages set forth herein may also be utilized, without departing from the spirit and scope of the present invention. Such modifications and variations are considered to be within the scope of the invention.

What is claimed is:

1. A method for producing fatty acid methyl esters comprising:
a. combining hydrogen, a carbon source selected from carbon monoxide and carbon dioxide, and a culture of microorganism cells, wherein said microorganism cells comprise a heterologous nucleic acid molecule encoding an O-methyltransferase protein; and
b. maintaining the combined hydrogen, carbon source, and microorganism cells for a suitable time and under conditions sufficient to convert the carbon source to fatty acid methyl esters.

2. The method of claim 1, wherein said carbon source has a ratio of carbon-14 to carbon-12 of about 1.0×10⁻¹⁴ or greater.

3. The method of claim 1, wherein said carbon source has a percentage of petroleum origin selected from less than about 50%, less than about 40%, less than about 30%, less than about 20%, less than about 10%, less than about 5%, less than about 1%, or essentially free of petroleum origin.

4. The method of claim 1, wherein said carbon source has an amount of glucose, sucrose, fructose, dextrose, lactose, xylose, arabinose, glycerol, and/or combinations thereof that is selected from the group consisting of less than about 50%, less than about 40%, less than about 30%, less than about 20%, less than about 10%, less than about 5%, and less than about 1% by weight.

5. The method of claim 1, wherein said method does not require the presence of a chemical catalyst for the conversion of the carbon source to fatty acid methyl esters.

6. The method of claim 1, wherein said fatty acid methyl esters include a mixture of fatty acid moieties.

7. The method of claim 1, wherein said microorganism cells further comprise a heterologous nucleic acid molecule encoding one or more proteins selected from the group consisting of phosphoglucose isomerase, inositol-1-phosphate synthase, inositol monophosphatase, myo-inositol dehydrogenase, myo-inosose-2-dehydratase, inositol 2-dehydrogenase, deoxy-D-gluconate isomerase, 5-dehydro-2-deoxygluconokinase, and deoxyphosphogluconate aldolase.

8. The method of claim 1, wherein said microorganism cells further comprise a heterologous nucleic acid molecule encoding one or more proteins selected from the group consisting of aldehyde dehydrogenase, malonyl-CoA synthetase, fatty acid synthetase complex, and fatty acyl-CoA/ACP thioesterase proteins.

9. The method of claim 1, further comprising processing said fatty acid methyl esters to conform to one or more ASTM diesel fuel oil blend standards.

10. The method of claim 1, wherein said method provides a higher yield of fatty acid methyl esters compared to an otherwise identical method with a microorganism lacking a heterologous nucleic acid molecule encoding an O-methyltransferase protein.

11. The method of claim 1, wherein the percentage of carbon source converted to fatty acid methyl esters is selected from greater than 25%, greater than 35%, greater than 45%, greater than 55%, greater than 65%, greater than 75%, greater than 85%, and greater than 95%.

12. The method of claim 1, wherein the volumetric productivity for fatty acid methyl esters is selected from at least 1/g/L/hr and at least 2/g/L/hr.

13. A method for producing malonate semialdehyde comprising:
a. combining hydrogen, a carbon source selected from carbon monoxide and carbon dioxide, and a culture of microorganism cells, wherein said microorganism cells comprise at least one genetic modification to introduce or increase one or more enzymatic activities selected from the group consisting of phosphoglucose isomerase, inositol-1-phosphate synthase, inositol monophosphatase, myo-inositol dehydrogenase, myo-inosose-2-dehydratase, inositol 2-dehydrogenase, deoxy-D-gluconate isomerase, 5-dehydro-2-deoxygluconokinase, and deoxyphophogluconate aldolase;
b. maintaining the combined hydrogen, carbon source, and microorganism cells for a suitable time and under conditions sufficient to convert the carbon source to malonate semialdehyde.

14. The method of claim 13, wherein said microorganism is capable of converting the carbon source to fructose-6-phosphate.

15. A method for producing an organic compound comprising
a. producing malonate semialdehyde according to claim 13;
b. further processing said malonate semialdehyde to yield the organic compound.

16. The method of claim 15, wherein said organic compound is fatty acid methyl ester.

17. The method of claim 13, wherein said microorganism cells further comprise a heterologous nucleic acid molecule encoding an O-methyltransferase protein.

18. The method of claim 13, wherein said microorganism cells further comprise a heterologous nucleic acid molecule encoding one or more proteins selected from the group consisting of aldehyde dehydrogenase, malonyl-CoA synthetase, fatty acid synthetase complex, and fatty acyl-CoA/ACP thioesterase proteins.

19. A method for producing malonate semialdehyde comprising:
a. combining hydrogen, a carbon source selected from carbon monoxide and carbon dioxide, and a culture of microorganism cells, wherein said microorganism cells comprise at least one genetic modification to introduce or increase one or more enzymatic activities selected from the group consisting of aldehyde dehydrogenase, malonyl-CoA synthetase, fatty acid synthase complex, and fatty acyl-CoA/ACP thioesterase proteins;
b. maintaining the combined hydrogen, carbon source, and microorganism cells for a suitable time and under conditions sufficient to convert the carbon source to malonate semialdehyde.

20. The method of claim 19, wherein said microorganism is capable of converting the carbon source to fructose-6-phosphate.

21. A method for producing an organic compound comprising
a. producing malonate semialdehyde according to claim 19;
b. further processing said malonate semialdehyde to yield the organic compound.

22. The method of claim 21, wherein said organic compound is fatty acid methyl ester.

23. The method of claim 19, wherein said microorganism cells further comprise a heterologous nucleic acid molecule encoding an O-methyltransferase protein.

24. A method for producing myo-inositol comprising:
a. combining hydrogen, a carbon source selected from carbon monoxide and carbon dioxide, and a culture of microorganism cells, wherein said microorganism cells comprise at least one genetic modification to introduce or increase one or more enzymatic activities selected from the group consisting of phosphoglucose isomerase, inositol-1-phosphate synthase, and inositol monophosphatase;
b. maintaining the combined hydrogen, carbon source, and microorganism cells for a suitable time and under conditions sufficient to convert the carbon source to myo-inositol.

25. The method of claim 24, wherein said microorganism is capable of converting the carbon source to fructose-6-phosphate.

26. A method for producing an organic compound comprising
a. producing myo-inositol according to claim 24;
b. further processing said myo-inositol to yield the organic compound.

27. The method of claim 26, wherein said organic compound is fatty acid methyl ester.

28. The method of claim 24, wherein said microorganism cells further comprise a heterologous nucleic acid molecule encoding an O-methyltransferase protein.

29. A genetically modified microorganism for the production of fatty acid methyl esters, wherein said microorganism comprises at least one heterologous nucleic acid molecule selected from the groups of nucleic acid molecules encoding
a. O-methyltransferase;
b. phosphoglucose isomerase, inositol-1-phosphate synthase, inositol monophosphatase, myo-inositol dehydrogenase, myo-inosose-2-dehydratase, inositol 2-dehydrogenase, deoxy-D-gluconate isomerase, 5-dehydro-2-deoxygluconokinase, deoxyphophogluconate aldolase, aldehyde dehydrogenase, malonyl-CoA synthetase, fatty acid synthase enzymes, and fatty acyl-CoA/ACP thioesterase; or
c. S-adenosyl-homocysteine hydrolase, ribonuclease hydrolase-3, homocycsteine transmethylase, and methionine adenosyltransferase.

30. The genetically modified microorganism of claim 29, wherein the number of genetic modifications is selected from at least two, at least three, at least four, at least five, at least six, at least seven, at least eight, at least nine, at least ten, at least eleven, and at least twelve enzymatic activities.

31. The genetically modified microorganism of claim 29, wherein said microorganism is selected from the group consisting of chemolithotrophic bacteria.

32. The genetically modified microorganism of claim 29, wherein said microorganism is selected from the group consisting Oligotropha carboxidovorans, Cupriavidus necator, and strain H16 of Cupriavidus necator.

33. The genetically modified microorganism of claim 29, wherein the heterologous nucleic acid molecule is selected from the group:
i) phosphoglucose isomerase encoded by the pgi gene of E. coli;
ii) inositol-1-phosphate synthase encoded by the ino-1 gene of S. cerevisiae;
iii) inositol monophosphatase encoded by the subB gene of E. coli;
iv) myo-inositol dehydrogenase encoded by the iolG gene of B. subtilis;
v) myo-inosose-2-dehydratase encoded by the iolE gene of B. subtilis;
vi) inositol 2-dehydrogenase encoded by the iolD gene of B. subtilis;
vii) deoxy-D-gluconate isomerase encoded by the iolB gene of B. subtilis;
viii) 5-dehydro-2-deoxygluconokinase encoded by the iolC gene of B. subtilis;
ix) deoxyphophogluconate aldolase encoded by the iolJ gene of B. subtilis;
x) aldehyde dehydrogenase encoded by the aldA gene of E. coli;
xi) malonyl-CoA synthetase encoded by the matB gene of R. leguminosum;
xii) a methyl-CoA-ACP transacetylase encoded by the fabD gene of E. coli;
xiii) an enzyme of the fatty acid synthase (cyclic elongation, saturated) complex encoded by fabF, fabH or fabB; fabG, fabA or fabZ, and fabI or fabK.
xiv) fatty acyl-CoA/ACP thioesterase encoded by the tesA gene of E. coli;
xv) S-adenosyl-homocysteine hydrolase encoded by the Ahcy gene of R. norvegicus;
xvi) ribonuclease hydrolase-3 is encoded by the rihC gene of E. coli;
xvii) homocycsteine transmethylase encoded by the metE gene of E. coli;
xviii) methionine adenosyltransferase encoded by the metK gene of E. coli; and
xix) O-methyltransferase encoded by the JHAMT gene of D. melanogaster.

34. The genetically modified microorganism of claim 29 comprising at least one genetic modification to introduce or increase one or more enzymatic activities provided by amino acid sequences having at least 50%, 60%, 70%, 80%, 85%, 90%, 92%, 95%, 96%, 97%, 98% or 99% sequence identity to one or more amino acid sequences selected from the group consisting of SEQ ID NO:002, SEQ ID NO:004, SEQ ID NO:006, SEQ ID NO:008, SEQ ID NO:010, SEQ ID NO:012, SEQ ID NO:014, SEQ ID NO:016, SEQ ID NO:018, SEQ ID NO:020, SEQ ID NO:022, SEQ ID NO:024, SEQ ID NO:026, and conservatively modified variants thereof.

35. The genetically modified microorganism of claim 29 comprising at least one genetic modification provided by a polynucleotide comprising a nucleic acid sequence having at least 50%, 60%, 70%, 80%, 85%, 90%, 92%, 95%, 96%, 97%, 98% or 99% sequence identity to one or more nucleic acid sequences from the group consisting of SEQ ID NO:001, SEQ ID NO:003, SEQ ID NO:005, SEQ ID NO:007, SEQ ID NO:009, SEQ ID NO:011, SEQ ID NO:013, SEQ ID NO:015, SEQ ID NO:017, SEQ ID NO:019, SEQ ID NO:021, SEQ ID NO:023, SEQ ID NO:025, and conservatively modified variants thereof.

36. The genetically modified microorganism of claim 29 wherein the heterologous nucleic acid molecule encoding the O-methyltransferase is selected from the group consisting of JHAMT Dm (Drosophila melanogaster), JHAMT tcMT3 (Tribolium castaneum), Putative JHAMT MT1 (Tribolium castaneum), Putative JHAMT tcMT2 (Tribolium castaneum), Mycobacterium smegmatis, str. MC2 155, methyltransferase, Cancer pagurus putative farnesoic acid O-methyltransferase, JHAMT Shrimp (Metapenaeus ensis), Ralstonia solanacearum UW5551 PhcB, and modified variants thereof.

37. A culture system comprising (i) a population of genetically modified microorganisms of claim 29, and (ii) a media comprising nutrients for said population.

38. A method of making a genetically modified microorganism according to claim 29 comprising providing to a microorganism at least one genetic modification to introduce or increase one or more enzymatic activities provided by amino acid sequences having at least 50%, 60%, 70%, 80%, 85%, 90%, 92%, 95%, 96%, 97%, 98% or 99% sequence identity to one or more amino acid sequences selected from the group consisting of SEQ ID NO:002, SEQ ID NO:004, SEQ ID NO:006, SEQ ID NO:008, SEQ ID NO:010, SEQ ID NO:012, SEQ ID NO:014, SEQ ID NO:016, SEQ ID NO:018, SEQ ID NO:020, SEQ ID NO:022, SEQ ID NO:024, SEQ ID NO:026, and conservatively modified variants thereof.

39. A method of making a genetically modified microorganism according to claim 29, comprising providing to a selected microorganism at least one genetic modification comprising through a polynucleotide comprising a nucleic acid sequence having at least 50%, 60%, 70%, 80%, 85%, 90%, 92%, 95%, 96%, 97%, 98% or 99% sequence identity to one or more nucleic acid sequences from the group consisting of SEQ ID NO:001, SEQ ID NO:003, SEQ ID NO:005, SEQ ID NO:007, SEQ ID NO:009, SEQ ID NO:011, SEQ ID NO:013, SEQ ID NO:015, SEQ ID NO:017, SEQ ID NO:019, SEQ ID NO:021, SEQ ID NO:023, SEQ ID NO:025, and conservatively modified variants thereof.


SEQUENCE LISTING (36 sequences)

SEQ ID NO: 1 - DNA, 1650 bp, Escherichia coli
atgaaaaacatcaatccaacgcagaccgctgcctggcaggcactacagaaacacttcgatgaaatgaaagacgttacgatcgccgatctttttgctaaagacggcgatcgtttttctaagttctccgcaaccttcgacgatcagatgctggtggattactccaaaaaccgcatcactgaagagacgctggcgaaattacaggatctggcgaaagagtgcgatctggcgggcgcgattaagtcgatgttctctggcgagaagatcaaccgcactgaaaaccgcgccgtgctgcacgtagcgctgcgtaaccgtagcaataccccgattttggttgatggcaaagacgtaatgccggaagtcaacgcggtgctggagaagatgaaaaccttctcagaagcgattatttccggtgagtggaaaggttataccggcaaagcaatcactgacgtagtgaacatcgggatcggcggttctgacctcggcccatacatggtgaccgaagctctgcgtccgtacaaaaaccacctgaacatgcactttgtttctaacgtcgatgggactcacatcgcggaagtgctgaaaaaagtaaacccggaaaccacgctgttcttggtagcatctaaaaccttcaccactcaggaaactatgaccaacgcccatagcgcgcgtgactggttcctgaaagcggcaggtgatgaaaaacacgttgcaaaacactttgcggcgctttccaccaatgccaaagccgttggcgagtttggtattgatactgccaacatgttcgagttctgggactgggttggcggccgttactctttgtggtcagcgattggcctgtcgattgttctctccatcggctttgataacttcgttgaactgctttccggcgcacacgcgatggacaagcatttctccaccacgcctgccgagaaaaacctgcctgtactgctggcgctgattggcatctggtacaacaatttctttggtgcggaaactgaagcgattctgccgtatgaccagtatatgcaccgtttcgcggcgtacttccagcagggcaatatggagtccaacggtaagtatgttgaccgtaacggtaacgttgtggattaccagactggcccgattatctggggtgaaccaggcactaacggtcagcacgcgttctaccagctgatccaccagggaaccaaaatggtaccgtgcgatttcatcgctccggctatcacccataacccgctctctgatcatcaccagaaactgctgtctaacttcttcgcccagaccgaagcgctggcgtttggtaaatcccgcgaagtggttgagcaggaatatcgtgatcagggtaaagatccggcaacgcttgactacgtggtgccgttcaaagtattcgaaggtaaccgcccgaccaactccatcctgctgcgtgaaatcactccgttcagcctgggtgcgttgattgcgctgtatgagcacaaaatctttactcagggcgtgatcctgaacatcttcaccttcgaccagtggggcgtggaactgggtaaacagctggcgaaccgtattctgccagagctgaaagatgataaagaaatcagcagccacgatagctcgaccaatggtctgattaaccgctataaagcgtggcgcggttaa

SEQ ID NO: 2 - protein, 549 aa, Escherichia coli
MKNINPTQTAAWQALQKHFDEMKDVTIADLFAKDGDRFSKFSATFDDQMLVDYSKNRITEETLAKLQDLAKECDLAGAIKSMFSGEKINRTENRAVLHVALRNRSNTPILVDGKDVMPEVNAVLEKMKTFSEAIISGEWKGYTGKAITDVVNIGIGGSDLGPYMVTEALRPYKNHLNMHFVSNVDGTHIAEVLKKVNPETTLFLVASKTFTTQETMTNAHSARDWFLKAAGDEKHVAKHFAALSTNAKAVGEFGIDTANMFEFWDWVGGRYSLWSAIGLSIVLSIGFDNFVELLSGAHAMDKHFSTTPAEKNLPVLLALIGIWYNNFFGAETEAILPYDQYMHRFAAYFQQGNMESNGKYVDRNGNVVDYQTGPIIWGEPGTNGQHAFYQLIHQGTKMVPCDFIAPAITHNPLSDHHQKLLSNFFAQTEALAFGKSREVVEQEYRDQGKDPATLDYVVPFKVFEGNRPTNSILLREITPFSLGALIALYEHKIFTQGVILNIFTFDQWGVELGKQLANRILPELKDDKEISSHDSSTNGLINRYKAWRG

SEQ ID NO: 3 - DNA, 1602 bp, Saccharomyces cerevisiae
atgctagaagataatattgctccaatcacctccgttaaagtagttaccgacaagtgcacgtacaaggacaacgagctgctcaccaagtacagctacgaaaatgctgtagttacgaagacagctagtggccgcttcgatgtaacgcccactgttcaagactacgtgttcaaacttgacttgaaaaagccggaaaaactaggaattatgctcattgggttaggtggcaacaatggctccactttagtggcctcggtattggcgaataagcacaatgtggagtttcaaactaaggaaggcgttaagcaaccaaactacttcggctccatgactcaatgttctaccttgaaactgggtatcgatgcggaggggaatgacgtttatgctccttttaactctctgttgcccatggttagcccaaacgactttgtcgtctctggttgggacatcaataacgcagatctatacgaagctatgcagagaagtcaagttctcgaatatgatctgcaacaacgcttgaaggcgaagatgtccttggtgaagcctcttccttccatttactaccctgatttcattgcagctaatcaagatgagagagccaataactgcatcaatttggatgaaaaaggcaacgtaaccacgaggggtaagtggacccatctgcaacgcatcagacgcgatatccagaatttcaaagaagaaaacgcccttgataaagtaatcgttctttggactgcaaatactgagaggtacgtagaagtatctcctggtgttaatgacaccatggaaaacctcttgcagtctattaagaatgaccatgaagagattgctccttccacgatctttgcagcagcatctatcttggaaggtgtcccctatattaatggttcaccgcagaatacttttgttcccggcttggttcagctggctgagcatgagggtacattcattgcgggagacgatctcaagtcgggacaaaccaagttgaagtctgttctggcccagttcttagtggatgcaggtattaaaccggtctccattgcatcctataaccatttaggcaataatgacggttataacttatctgctccaaaacaatttaggtctaaggagatttccaaaagttctgtcatagatgacatcatcgcgtctaatgatatcttgtacaatgataaactgggtaaaaaagttgaccactgcattgtcatcaaatatatgaagcccgtcggggactcaaaagtggcaatggacgagtattacagtgagttgatgttaggtggccataaccggatttccattcacaatgtttgcgaagattctttactggctacgcccttgatcatcgatcttttagtcatgactgagttttgtacaagagtgtcctataagaaggtggacccagttaaagaagatgctggcaaattcgagaacttttatccagttttaaccttcttgagttactggttaaaagctccattaacaagaccaggatttcacccggtgaatggcttaaacaagcaaagaaccgccttagaaaattttttaagattgttgattggattgccttctcaaaacgaactaagattcgaagagagattgttgtaa

SEQ ID NO: 4 - protein, 533 aa, Saccharomyces cerevisiae
MLEDNIAPITSVKVVTDKCTYKDNELLTKYSYENAVVTKTASGRFDVTPTVQDYVFKLDLKKPEKLGIMLIGLGGNNGSTLVASVLANKHNVEFQTKEGVKQPNYFGSMTQCSTLKLGIDAEGNDVYAPFNSLLPMVSPNDFVVSGWDINNADLYEAMQRSQVLEYDLQQRLKAKMSLVKPLPSIYYPDFIAANQDERANNCINLDEKGNVTTRGKWTHLQRIRRDIQNFKEENALDKVIVLWTANTERYVEVSPGVNDTMENLLQSIKNDHEEIAPSTIFAAASILEGVPYINGSPQNTFVPGLVQLAEHEGTFIAGDDLKSGQTKLKSVLAQFLVDAGIKPVSIASYNHLGNNDGYNLSAPKQFRSKEISKSSVIDDIIASNDILYNDKLGKKVDHCIVIKYMKPVGDSKVAMDEYYSELMLGGHNRISIHNVCEDSLLATPLIIDLLVMTEFCTRVSYKKVDPVKEDAGKFENFYPVLTFLSYWLKAPLTRPGFHPVNGLNKQRTALENFLRLLIGLPSQNELRFEERLL

SEQ ID NO: 5 - DNA, 804 bp, Escherichia coli
atgcatccgatgctgaacatcgccgtgcgcgcagcgcgcaaggcgggtaatttaattgccaaaaactatgaaaccccggacgctgtagaagcgagccagaaaggcagtaacgatttcgtgaccaacgtagataaagctgccgaagcggtgattatcgacacgattcgtaaatcttacccacagcacaccatcatcaccgaagaaagcggtgaacttgaaggtactgatcaggatgttcaatgggttatcgatccactggatggcactaccaactttatcaaacgtctgccgcacttcgcggtatctatcgctgttcgtatcaaaggccgcaccgaagttgctgtggtatacgatcctatgcgtaacgaactgttcaccgccactcgcggtcagggcgcacagctgaacggctaccgactgcgcggcagcaccgctcgcgatctcgacggtactattctggcgaccggcttcccgttcaaagcaaaacagtacgccactacctacatcaacatcgtcggcaaactgttcaacgaatgtgcagacttccgtcgtaccggttctgcggcgctggatctggcttacgtcgctgcgggtcgtgttgacggtttctttgaaatcggtctgcgcccgtgggacttcgccgcaggcgagctgctggttcgtgaagcgggcggcatcgtcagcgacttcaccggtggtcataactacatgctgaccggtaacatcgttgctggtaacccgcgcgttgttaaagccatgctggcgaacatgcgtgacgagttaagcgacgctctgaagcgttaa

SEQ ID NO: 6 - protein, 267 aa, Escherichia coli
MHPMLNIAVRAARKAGNLIAKNYETPDAVEASQKGSNDFVTNVDKAAEAVIIDTIRKSYPQHTIITEESGELEGTDQDVQWVIDPLDGTTNFIKRLPHFAVSIAVRIKGRTEVAVVYDPMRNELFTATRGQGAQLNGYRLRGSTARDLDGTILATGFPFKAKQYATTYINIVGKLFNECADFRRTGSAALDLAYVAAGRVDGFFEIGLRPWDFAAGELLVREAGGIVSDFTGGHNYMLTGNIVAGNPRVVKAMLANMRDELSDALKR

SEQ ID NO: 7 - DNA, 1035 bp, Bacillus subtilis
atgagtttacgtattggcgtaattggaactggagcaatcggaaaagaacatattaaccgtatcacgaacaagctgtcaggcgcggaaattgtagctgtaacggatgttaatcaagaagctgcacaaaaggtcgttgagcaataccaattaaacgcgacggtttatccgaatgatgacagcttgcttgcagacgaaaatgtagacgctgttttagtgacaagctgggggcctgcgcatgagtcaagcgtgctgaaagcgattaaagcccagaaatatgtgttctgtgaaaaaccgctcgcgacaacggctgaaggatgcatgcgcattgtcgaagaagaaatcaaagtgggcaaacgccttgttcaagtcggcttcatgcgccgttatgacagcggttacgtacagctgaaagaagcgctcgataatcatgtcaacggcgagcctcttatgattcactgcgcgcaccgcaacccgactgtaggagataactatacaacggatatggctgtagtcgacacgcttgttcatgaaattgacgtgctccactggctcgtcaatgatgactacgagtccgttcaagtcatctatccgaaaaaatcaaaaaacgcgcttccacatttaaaagatccgcaaatcgtcgtgattgaaacaaaaggcggtatcgtcatcaatgctgaaatctatgtgaactgtaaatacggctatgacattcaatgtgaaatcgtcggagaagacggcatcatcaagcttcccgagccatcaagcatcagcttgagaaaagaaggcagattcagcactgatattttgatggattggcagagacgctttgtcgctgcgtatgatgtggaaatccaagactttattgattcgattcaaaagaaaggcgaggtcagcggaccgacggcatgggacggctatattgctgctgtcacgactgacgcgtgtgtaaaagcccaggaatctggacaaaaagaaaaggttgaattgaaggaaaaaccggaattctatcaatcttttacaacagttcaaaactaa

SEQ ID NO: 8 - protein, 344 aa, Bacillus subtilis
MSLRIGVIGTGAIGKEHINRITNKLSGAEIVAVTDVNQEAAQKVVEQYQLNATVYPNDDSLLADENVDAVLVTSWGPAHESSVLKAIKAQKYVFCEKPLATTAEGCMRIVEEEIKVGKRLVQVGFMRRYDSGYVQLKEALDNHVNGEPLMIHCAHRNPTVGDNYTTDMAVVDTLVHEIDVLHWLVNDDYESVQVIYPKKSKNALPHLKDPQIVVIETKGGIVINAEIYVNCKYGYDIQCEIVGEDGIIKLPEPSSISLRKEGRFSTDILMDWQRRFVAAYDVEIQDFIDSIQKKGEVSGPTAWDGYIAAVTTDACVKAQESGQKEKVELKEKPEFYQSFTTVQN

SEQ ID NO: 9 - DNA, 894 bp, Bacillus subtilis
atgggcaaaaatgaaatcctgtggggaatcgctcccattgggtggcggaatgatgacatgcctgaaattggagcgggaaatacacttcagcatttgttaagtgatatcgttgtcgcacgttttcaaggcacggaggtcgggggctttttccccgaacctgccatcctgaacaaagagctgaagcttcggaacttacgcattgcaggaaaatggttcagcagttttattttgcgtgacggacttggtgaagcggcaaagacatttaccctgcattgtgagtatttgcagcaagtaaacgcggatgtcgcagttgtctctgaacaaacgtacagcgtgcaaagcttggagaaaaatgtgttcacagagaagccgcactttacggatgatgaatgggagcggctttgcgaagggctgaatcaccttggcgaaattgccgctcagcatggcttgaagcttgtctatcatcatcatctcggcactggtgtccaaacagcggaagaagtggaccgcctgatggcaggaacagaccctgcgcatgtacacctcctctatgatacaggccatgcgtatatttctgacggcgattacatggggatgcttgagaagcatatcggccgcattaagcatgtgcactttaaggatgcccgcctgaatgtcatggaacaatgcaggctcgaaggacaatcgttccggcaatcatttttaaaaggcatgtttacggttcccggtgacggctgcattgactttagagaagtatatcagctgctgttgaagcacagttattccggatggattgtcattgaagctgaacaagaccccgatgttgcaaacccgctggagtatgcattgattgcgagaaactatattgatcagcagttgttggatctggcttaa

SEQ ID NO: 10 - protein, 297 aa, Bacillus subtilis
MGKNEILWGIAPIGWRNDDMPEIGAGNTLQHLLSDIVVARFQGTEVGGFFPEPAILNKELKLRNLRIAGKWFSSFILRDGLGEAAKTFTLHCEYLQQVNADVAVVSEQTYSVQSLEKNVFTEKPHFTDDEWERLCEGLNHLGEIAAQHGLKLVYHHHLGTGVQTAEEVDRLMAGTDPAHVHLLYDTGHAYISDGDYMGMLEKHIGRIKHVHFKDARLNVMEQCRLEGQSFRQSFLKGMFTVPGDGCIDFREVYQLLLKHSYSGWIVIEAEQDPDVANPLEYALIARNYIDQQLLDLA

SEQ ID NO: 11 - DNA, 1710 bp, Bacillus subtilis
atggcgcatgcggccatggcgtacagcaagcaaatgctgagaagaaaaatatatgcggtgtctacatccgtcggacctggagcggctaacttggtggcggcagccggcactgcattggcaaataatatcccagttctcttgattccggcagacacatttgcgacaagacagccagaccctgtactgcagcaaatggagcaagaatacagtgcggcgattacgacaaacgatgcattgaagcctgtatcgagatactgggaccgcattacgcgccctgagcagctgatgagcagtttgctgcgcgcgtttgaagtcatgactgatccggcaaaagcaggtccggcaacgatttgtatttctcaggatgttgaaggggaagcatacgattttgatgaaagtttcttcgtcaaacgggttcactatattgatcgcatgcagccgagcgagcgcgagcttcaaggtgcggcggagctgattaaatccagcaaaaaacctgtgattctcgtcggcggaggcgcaaaatattccggtgcgcgcgatgaattggttgccatttctgaagcctataacattccgttagttgaaacgcaggcaggaaagtctaccgttgaggcagattttgcgaacaaccttggcggaatgggcatcacaggtacacttgcggcaaacaaagcggcccgccaagctgatttgattatcggcatcggcacaaggtatacagattttgcgacatcctctaagaccgcttttgattttgataaagcgaagtttttgaacattaacgtcagccgaatgcaggcgtataaacttgatgcattccaagtggtggcggatgcgaaagtgacgcttggcaaactgcacggcttgcttgaaggctatgagagcgagttcggcacaacgatccgggagctgaaggacgaatggctagctgaacgcgagcgtctcagcaaagtgacgtttaagcgggaagcatttgatccggaaatcaaaaatcacttttctcaagaggtcctgaatgaatatgctgacgcgttgaatacggagcttccgcaaacaacggcattgctgacaatcaacgagacgattcctgaagacagcgtcatcatctgttcagcaggctcactcccaggagatttgcagcgtctgtggcattctaatgttccgaatacgtatcacctggagtatggatattcttgcatgggctatgaagtgtccgggacactcggtcttaaactggcccaccctgacagagaagtgtattcaatcgtcggagacggcagcttcctgatgcttcattctgaactgatcacggcgattcagtacaacaagaaaatcaatgtgctgctctttgacaactcaggattcggatgcatcaacaacctgcaaatggatcacggcagcggcagctactattgcgagttccgcacagatgacaaccaaatcctgaatgtcgattacgcgaaagtcgctgagggatacggcgcgaaaacctaccgtgcaaacacagtagaagaattaaaagctgcgttagaggatgcgaagaaacaggatgtatcaacattaattgaaatgaaggtgctgcctaaaacaatgacggacggctatgacagctggtggcatgtcggggtggcagaggtatctgaacaagaaagcgttcagaaagcatacgaagcaaaagagaaaaagctggaatctgcgaagcagtattag

SEQ ID NO: 12 - protein, 569 aa, Bacillus subtilis
MAHAAMAYSKQMLRRKIYAVSTSVGPGAANLVAAAGTALANNIPVLLIPADTFATRQPDPVLQQMEQEYSAAITTNDALKPVSRYWDRITRPEQLMSSLLRAFEVMTDPAKAGPATICISQDVEGEAYDFDESFFVKRVHYIDRMQPSERELQGAAELIKSSKKPVILVGGGAKYSGARDELVAISEAYNIPLVETQAGKSTVEADFANNLGGMGITGTLAANKAARQADLIIGIGTRYTDFATSSKTAFDFDKAKFLNINVSRMQAYKLDAFQVVADAKVTLGKLHGLLEGYESEFGTTIRELKDEWLAERERLSKVTFKREAFDPEIKNHFSQEVLNEYADALNTELPQTTALLTINETIPEDSVIICSAGSLPGDLQRLWHSNVPNTYHLEYGYSCMGYEVSGTLGLKLAHPDREVYSIVGDGSFLMLHSELITAIQYNKKINVLLFDNSGFGCINNLQMDHGSGSYYCEFRTDDNQILNVDYAKVAEGYGAKTYRANTVEELKAALEDAKKQDVSTLIEMKVLPKTMTDGYDSWWHVGVAEVSEQESVQKAYEAKEKKLESAKQY

SEQ ID NO: 13 - DNA, 816 bp, Bacillus subtilis
atgagttatttgttgcgtaagccgcagtcgcatgaagtgtctaatggggtcaaactcgtgcacgaagtaacgacatccaactctgatctcacttatgtagagtttaaagtgttagatcttgcatcaggttcaagctatacagaagaattgaaaaaacaagaaatctgtattgtggcggtaacggggaaaattacagtgacagatcatgagtcgacttttgagaatatcggcacgcgcgaaagctattttgaacgaaaaccgacagacagcgtctatatttcaaatgaccgtgcatttgagatcacagcggtcagcgacgcaagagtggcgctttgctattctccatcggaaaagcagcttccgacaaagctgatcaaagcggaagacaacggaattgagcatcgcgggcaattttcaaacaaacgtactgttcataacattcttccggattcagacccttcagctaacagtctattagtagttgaagtctatacagacagcggcaactggtccagctacccgcctcacaaacatgaccaagacaacttgccggaagaatctttcttagaagaaacgtactaccatgagttagacccgggacagggctttgtgtttcagcgcgtatacacagatgaccgttctattgacgagacaatgactgtgggaaatgaaaacgttgtcatcgttcctgcgggataccacccggtaggcgttccggacggatacacatcctactatttaaatgtcatggcagggccgacgcgaaaatggaagttttataacgacccggcgcatgaatggattttagaacgctaa

SEQ ID NO: 14 - protein, 271 aa, Bacillus subtilis
MSYLLRKPQSHEVSNGVKLVHEVTTSNSDLTYVEFKVLDLASGSSYTEELKKQEICIVAVTGKITVTDHESTFENIGTRESYFERKPTDSVYISNDRAFEITAVSDARVALCYSPSEKQLPTKLIKAEDNGIEHRGQFSNKRTVHNILPDSDPSANSLLVVEVYTDSGNWSSYPPHKHDQDNLPEESFLEETYYHELDPGQGFVFQRVYTDDRSIDETMTVGNENVVIVPAGYHPVGVPDGYTSYYLNVMAGPTRKWKFYNDPAHEWILER

SEQ ID NO: 15 - DNA, 1020 bp, Bacillus subtilis
atggattttagaacgctaacaagtgaggagtggctgtttacgatgaagtatacattcaatgaagagaaggcttttgatattgttgccatcggccgggcatgtattgatctgaacgcagtcgaatacaaccgcccaatggaagaaacgatgacattttcgaaatatgtcggcggttcacctgcgaatatcgcgatcggcagcgcgaagcttggcttaaaagcgggcttcatcggcaaaattccggatgaccagcatggaagattcatagagtcctatatgagaaagaccggcgtggatactacacagatgattgttgatcaagatggacacaaagcaggccttgcatttacagaaatcctcagccctgaagaatgcagcatcttaatgtatcgcgatgatgtggcggatctttatcttgagccttcagaggtaagtgaggactatatcgcaaatgcgaaaatgctgcttgtctccgggacagcgctcgccaaaagcccgtcacgggaagcggtgttaaaagctgttcaatacgcgaaaaagcatcaggttaaggtggtattcgaactggattaccggccatatacgtggcagtcatcagatgaaacagccgtttattattctttggttgccgagcagtctgatatcgtcatcggcacacgcgatgaatttgatgtgatggaaaaccgcacaggcggaagcaatgaagaatccgtcaatcatttatttggccattcagccgacctcgttgtcatcaaacacggcgtcgaaggctcttacgcatacagcaaatccggcgaggtattccgcgctcaagcgtacaagacaaaagtgctgaaaacctttggggccggtgactcctatgcgtcagcctttatctatggccttgtcagcggaaaagacattgaaacggcattgaaatacggcagtgcttcagcctccattgtggtgagcaagcacagttcgtcagaagcgatgccgactgcggaagaaatcgaacagcttattgaagcacagtcataa

SEQ ID NO: 16 - protein, 339 aa, Bacillus subtilis
MDFRTLTSEEWLFTMKYTFNEEKAFDIVAIGRACIDLNAVEYNRPMEETMTFSKYVGGSPANIAIGSAKLGLKAGFIGKIPDDQHGRFIESYMRKTGVDTTQMIVDQDGHKAGLAFTEILSPEECSILMYRDDVADLYLEPSEVSEDYIANAKMLLVSGTALAKSPSREAVLKAVQYAKKHQVKVVFELDYRPYTWQSSDETAVYYSLVAEQSDIVIGTRDEFDVMENRTGGSNEESVNHLFGHSADLVVIKHGVEGSYAYSKSGEVFRAQAYKTKVLKTFGAGDSYASAFIYGLVSGKDIETALKYGSASASIVVSKHSSSEAMPTAEEIEQLIEAQS

SEQ ID NO: 17 - DNA, 894 bp, Bacillus subtilis
atggataaaggaggggtgaatatggcttttgtatcgatgaaagagcttcttgaagatgcaaagcgggagcaatatgcaattggccagtttaatatcaacggcctgcaatggacgaaggcgattttgcaggcggcgcaaaaggagcaatcaccggtcatcgccgcggcttccgatcgcctggtcgactatttaggcggatttaaaacgattgccgccatggtcggcgcgttaatagaggacatggcgattaccgttccggtcgtgcttcatctcgatcacggcagcagtgcggaacgctgcagacaggccattgatgccggattcagctcagtgatgattgacggctcccatcagccgattgacgagaatatcgcgatgacaaaagaagtcaccgattatgccgcaaaacacggcgtgtcagtagaagccgaagtcggcacggtcggcggaatggaagacggactggtcggcggggtccgctatgcggatatcacggaatgtgagcggatcgttaaagaaaccaatatcgacgcgctggccgccgccctcggctctgtacacggcaaatatcagggtgagccgaatctcggatttaaggaaatggaggctatctcccgcatgactgatattcccctcgttcttcacggggcatccgggattccgcaggatcagatcaaaaaagccatcacgctcggccacgcgaagatcaatatcaatacggaatgtatggtagcgtggacagacgaaacacgccgcatgtttcaggaaaacagcgatctgtacgaaccgcgcggctatttgacacccggcattgaagccgtggaagagacagtgcgaagcaaaatgagagagttcggatcagccggtaaagcagctaagcagcaggtcggctaa

SEQ ID NO: 18 - protein, 297 aa, Bacillus subtilis
MDKGGVNMAFVSMKELLEDAKREQYAIGQFNINGLQWTKAILQAAQKEQSPVIAAASDRLVDYLGGFKTIAAMVGALIEDMAITVPVVLHLDHGSSAERCRQAIDAGFSSVMIDGSHQPIDENIAMTKEVTDYAAKHGVSVEAEVGTVGGMEDGLVGGVRYADITECERIVKETNIDALAAALGSVHGKYQGEPNLGFKEMEAISRMTDIPLVLHGASGIPQDQIKKAITLGHAKININTECMVAWTDETRRMFQENSDLYEPRGYLTPGIEAVEETVRSKMREFGSAGKAAKQQVG

SEQ ID NO: 19 - DNA, 1440 bp, Escherichia coli
atgtcagtacccgttcaacatcctatgtatatcgatggacagtttgttacctggcgtggagacgcatggattgatgtggtaaaccctgctacagaggctgtcatttcccgcatacccgatggtcaggccgaggatgcccgtaaggcaatcgatgcagcagaacgtgcacaaccagaatgggaagcgttgcctgctattgaacgcgccagttggttgcgcaaaatctccgccgggatccgcgaacgcgccagtgaaatcagtgcgctgattgttgaagaagggggcaagatccagcagctggctgaagtcgaagtggcttttactgccgactatatcgattacatggcggagtgggcacggcgttacgagggcgagattattcaaagcgatcgtccaggagaaaatattcttttgtttaaacgtgcgcttggtgtgactaccggcattctgccgtggaacttcccgttcttcctcattgcccgcaaaatggctcccgctcttttgaccggtaataccatcgtcattaaacctagtgaatttacgccaaacaatgcgattgcattcgccaaaatcgtcgatgaaataggccttccgcgcggcgtgtttaaccttgtactggggcgtggtgaaaccgttgggcaagaactggcgggtaacccaaaggtcgcaatggtcagtatgacaggcagcgtctctgcaggtgagaagatcatggcgactgcggcgaaaaacatcaccaaagtgtgtctggaattggggggtaaagcaccagctatcgtaatggacgatgccgatcttgaactggcagtcaaagccatcgttgattcacgcgtcattaatagtgggcaagtgtgtaactgtgcagaacgtgtttatgtacagaaaggcatttatgatcagttcgtcaatcggctgggtgaagcgatgcaggcggttcaatttggtaaccccgctgaacgcaacgacattgcgatggggccgttgattaacgccgcggcgctggaaagggtcgagcaaaaagtggcgcgcgcagtagaagaaggggcgagagtggcgttcggtggcaaagcggtagaggggaaaggatattattatccgccgacattgctgctggatgttcgccaggaaatgtcgattatgcatgaggaaacctttggcccggtgctgccagttgtcgcatttgacacgctggaagatgctatctcaatggctaatgacagtgattacggcctgacctcatcaatctatacccaaaatctgaacgtcgcgatgaaagccattaaagggctgaagtttggtgaaacttacatcaaccgtgaaaacttcgaagctatgcaaggcttccacgccggatggcgtaaatccggtattggcggcgcagatggtaaacatggcttgcatgaatatctgcagacccaggtggtttatttacagtcttaa

SEQ ID NO: 20 - protein, 479 aa, Escherichia coli
MSVPVQHPMYIDGQFVTWRGDAWIDVVNPATEAVISRIPDGQAEDARKAIDAAERAQPEWEALPAIERASWLRKISAGIRERASEISALIVEEGGKIQQLAEVEVAFTADYIDYMAEWARRYEGEIIQSDRPGENILLFKRALGVTTGILPWNFPFFLIARKMAPALLTGNTIVIKPSEFTPNNAIAFAKIVDEIGLPRGVFNLVLGRGETVGQELAGNPKVAMVSMTGSVSAGEKIMATAAKNITKVCLELGGKAPAIVMDDADLELAVKAIVDSRVINSGQVCNCAERVYVQKGIYDQFVNRLGEAMQAVQFGNPAERNDIAMGPLINAAALERVEQKVARAVEEGARVAFGGKAVEGKGYYYPPTLLLDVRQEMSIMHEETFGPVLPVVAFDTLEDAISMANDSDYGLTSSIYTQNLNVAMKAIKGLKFGETYINRENFEAMQGFHAGWRKSGIGGADGKHGLHEYLQTQVVYLQS

SEQ ID NO: 21 - DNA, 1515 bp, Rhizobium leguminosarum
gtgagcaaccatcttttcgacgccatgcgggccgccgcgcccggtaacgcaccattcatccggatcgataacacgcgcacatggacctatgacgacgccttcgctctttccggccgcattgccagcgcgatggacgcgctcggcattcgccccggcgaccgcgttgcggtgcaggtcgagaaaagtgccgaggcattgatcctctatctcgcctgtcttcgaagcggcgccgtctacctgccgctcaacaccgcctatacgctggctgagctcgattattttatcggcgatgcggagccgcgtttggtggttgtcgcatcgtcggctcgagcgggcgtggagacaatcgccaagccccgcggtgcgatcgtcgaaactctcgacgctgctggcagcggctcgttgctggatctcgcccgcgacgagccggccgactttgtcgatgcctcgcgctccgccgatgatctggcggcgatcctctacacgtccggaacgacgggacgctccaagggggcgatgctcacgcatgggaacctgctctcgaacgccctgaccttgcgagatttttggcgcgtcaccgccggcgatcgactgatccatgccttgccgatcttccacacgcatggactgttcgtcgccacgaacgtcacactgctcgccggcgcctcgatgttcctgctgtcgaagttcgacccggaggagatcctgtcgctgatgccgcaggcaacgatgctgatgggcgtgccgaccttctacgtgcgcctcctgcagagcccgcgcctcgacaagcaagcggtcgccaacatccgcctcttcatttccggttcggctccactgcttgcagaaacacataccgagttccaggcacgtaccggtcacgccattctcgagcgctacggcatgacggaaaccaatatgaacacgtccaacccttatgaggggaaacggattgccggaacggtcggcttcccgctgcctgatgtgacggtgcgcgtcaccgatcccgccaccgggctcgcgctgccgcccgaacaaaccggcatgatcgagatcaaggggccgaacgttttcaagggctattggcgcatgcccgaaaaaaccgcggccgaattcaccgccgacggtttcttcatcagcggcgatctcggcaagatcgaccgcgacggttatgtccacatcgtcggccgcggcaaggatctggtgatttcgggtggatacaacatctatccgaaagaggttgagggcgagatcgaccagatcgagggtgtggttgagagcgctgtgatcggcgtgccgcatcccgatttcggagaaggcgtaacggccgtcgtcgtgcgcaagcccggcgctgccctcgatgaaaaggccatcgtcagcgccctccaggaccggctcgcgcgctacaaacaacccaagcgcatcatctttgcagaggacttgccgcgcaacacgatgggtaaggttcagaaaaacatcctgcggcagcaatacgccgatctttataccaggacgtaa

SEQ ID NO: 22 - protein, 504 aa, Rhizobium leguminosarum
MSNHLFDAMRAAAPGNAPFIRIDNTRTWTYDDAFALSGRIASAMDALGIRPGDRVAVQVEKSAEALILYLACLRSGAVYLPLNTAYTLAELDYFIGDAEPRLVVVASSARAGVETIAKPRGAIVETLDAAGSGSLLDLARDEPADFVDASRSADDLAAILYTSGTTGRSKGAMLTHGNLLSNALTLRDFWRVTAGDRLIHALPIFHTHGLFVATNVTLLAGASMFLLSKFDPEEILSLMPQATMLMGVPTFYVRLLQSPRLDKQAVANIRLFISGSAPLLAETHTEFQARTGHAILERYGMTETNMNTSNPYEGKRIAGTVGFPLPDVTVRVTDPATGLALPPEQTGMIEIKGPNVFKGYWRMPEKTAAEFTADGFFISGDLGKIDRDGYVHIVGRGKDLVISGGYNIYPKEVEGEIDQIEGVVESAVIGVPHPDFGEGVTAVVVRKPGAALDEKAIVSALQDRLARYKQPKRIIFAEDLPRNTMGKVQKNILRQQYADLYTRT

SEQ ID NO: 23 - protein, 309 aa, Escherichia coli [sequence omitted]

SEQ ID NO: 24 - protein, 317 aa, Escherichia coli
MYTKIIGTGSYLPEQVRTNADLEKMVDTSDEWIVTRTGIRERHIAAPNETVSTMGFEAATRAIEMAGIEKDQIGLIVVATTSATHAFPSAACQIQSMLGIKGCPAFDVAAACAGFTYALSVADQYVKSGAVKYALVVGSDVLARTCDPTDRGTIIIFGDGAGAAVLAASEEPGIISTHLHADGSYGELLTLPNADRVNPENSIHLTMAGNEVFKVAVTELAHIVDETLAANNLDRSQLDWLVPHQANLRIISATAKKLGMSMDNVVVTLDRHGNTSAASVPCALDEAVRDGRIKPGQLVLLEAFGGGFTWGSALVRF

SEQ ID NO: 25 - protein, 244 aa, Escherichia coli
MNFEGKIALVTGASRGIGRAIAETLAARGAKVIGTATSENGAQAISDYLGANGKGLMLNVTDPASIESVLEKIRAEFGEVDILVNNAGITRDNLLMRMKDEEWNDIIETNLSSVFRLSKAVMRAMMKKRHGRIITIGSVVGTMGNGGQANYAAAKAGLIGFSKSLAREVASRGITVNVVAPGFIETDMTRALSDDQRAGILAQVPAGRLGGAQEIANAVAFLASDEAAYITGETLHVNGGMYMV

SEQ ID NO: 26 - protein, 151 aa, Escherichia coli
MTTNTHTLQIEEILELLPHRFPFLLVDRVLDFEEGRFLRAVKNVSVNEPFFQGHFPGKPIFPGVLILEAMAQATGILAFKSVGKLEPGELYYFAGIDEARFKRPVVPGDQMIMEVTFEKTRRGLTRFKGVALVDGKVVCEATMMCARSREA

SEQ ID NO: 27 - protein, 262 aa, Escherichia coli
MGFLSGKRILVTGVASKLSIAYGIAQAMHREGAELAFTYQNDKLKGRVEEFAAQLGSDIVLQCDVAEDASIDTMFAELGKVWPKFDGFVHSIGFAPGDQLDGDYVNAVTREGFKIAHDISSYSFVAMAKACRSMLNPGSALLTLSYLGAERAIPNYNVMGLAKASLEANVRYMANAMGPEGVRVNAISAGPIRTLAASGIKDFRKMLAHCEAVTPIRRTVTIEDVGNSAAFLCSDLSAGISGEVVHVDGGFSIAAMNELELK

SEQ ID NO: 28 - protein, 314 aa, Carboxydothermus hydrogenoformans
MKTKITELLKIKYPIIQGGMAWVATARLAAAVSNAGGLGIIGAGNAPAEWVLAEVRKVKNLTDKPFGVNVMLLSPHVDEVMEVIIEEKVPVITTGAGNPGKYIKKLKENNVKIIPVVASVALAKRLEKTGVDAVIAEGHESGGHIGELTTMALVPQVVDNVSIPVVAAGGIADGRGLVAALALGAQAVQIGTRFLCAEETEIHPAVKEAVIKAGDRDTVITGASTGHPVRVIKNKLARRFLELEQKGAPPEELEKLGAGSLRRCMQEGDIEEGSLMAGQIAGLIKEIKPVKEIIEEIMHEAREIMKRIVREFDE

SEQ ID NO: 29 - protein, 413 aa, Escherichia coli
MSKRRVVVTGLGMLSPVGNTVESTWKALLAGQSGISLIDHFDTSAYATKFAGLVKDFNCEDIISRKEQRKMDAFIQYGIVAGVQAMQDSGLEITEENATRIGAAIGSGIGGLGLIEENHTSLMNGGPRKISPFFVPSTIVNMVAGHLTIMYGLRGPSISIATACTSGVHNIGHAARIIAYGDADVMVAGGAEKASTPLGVGGFGAARALSTRNDNPQAASRPWDKERDGFVLGDGAGMLVLEEYEHAKKRGAKIYAELVGFGMSSDAYHMTSPPENGAGAALAMANALRDAGIEASQIGYVNAHGTSTPAGDKAEAQAVKTIFGEAASRVLVSSTKSMTGHLLGAAGAVESIYSILALRDQAVPPTINLDNPDEGCDLDFVPHEARQVSGMEYTLCNSFGFGGTNGSLIFKKI

SEQ ID NO: 30 - protein, 406 aa, Escherichia coli
MKRAVITGLGIVSSIGNNQQEVLASLREGRSGITFSQELKDSGMRSHVWGNVKLDTTGLIDRKVVRFMSDASIYAFLSMEQAIADAGLSPEAYQNNPRVGLIAGSGGGSPRFQVFGADAMRGPRGLKAVGPYVVTKAMASGVSACLATPFKIHGVNYSISSACATSAHCIGNAVEQIQLGKQDIVFAGGGEELCWEMACEFDAMGALSTKYNDTPEKASRTYDAHRDGFVIAGGGGMVVVEELEHALARGAHIYAEIVGYGATSDGADMVAPSGEGAVRCMKMAMHGVDTPIDYLNSHGTSTPVGDVKELAAIREVFGDKSPAISATKAMTGHSLGAAGVQEAIYSLLMLEHGFIAPSINIEELDEQAAGLNIVTETTDRELTTVMSNSFGFGGTNATLVMRKLKD

SEQ ID NO: 31 - DNA, 627 bp, Escherichia coli
atgatgaacttcaacaatgttttccgctggcatttgcccttcctgttcctggtcctgttaaccttccgtgccgccgcagcggacacgttattgattctgggtgatagcctgagcgccgggtatcgaatgtctgccagcgcggcctggcctgccttgttgaatgataagtggcagagtaaaacgtcggtagttaatgccagcatcagcggcgacacctcgcaacaaggactggcgcgccttccggctctgctgaaacagcatcagccgcgttgggtgctggttgaactgggcggcaatgacggtttgcgtggttttcagccacagcaaaccgagcaaacgctgcgccagattttgcaggatgtcaaagccgccaacgctgaaccattgttaatgcaaatacgtctgcctgcaaactatggtcgccgttataatgaagcctttagcgccatttaccccaaactcgccaaagagtttgatgttccgctgctgcccttttttatggaagaggtctacctcaagccacaatggatgcaggatgacggtattcatcccaaccgcgacgcccagccgtttattgccgactggatggcgaagcagttgcagcctttagtaaatcatgactcataa

SEQ ID NO: 32 - protein, 208 aa, Escherichia coli
MMNFNNVFRWHLPFLFLVLLTFRAAAADTLLILGDSLSAGYRMSASAAWPALLNDKWQSKTSVVNASISGDTSQQGLARLPALLKQHQPRWVLVELGGNDGLRGFQPQQTEQTLRQILQDVKAANAEPLLMQIRLPANYGRRYNEAFSAIYPKLAKEFDVPLLPFFMEEVYLKPQWMQDDGIHPNRDAQPFIADWMAKQLQPLVNHDS

SEQ ID NO: 33 - protein, 467 aa, Ralstonia solanacearum
MYSPNQIDPAVSFRNSQGQQVRGTIITLQRRALVMEVYNPYSIVQVSEVLSDLAIKMGTRQAYLGKAVVVSLVNTGLTAVVSVTLTEEWRGLADVQDSPKLVGEEARAFVQDWEERFRIRHDYQIVVNEMRAFLAEVSRWVEQVDLSDSLPKEGENRLRLDVFQELAEPITLKVKYFQDWLESKAADVEPELAPAHRSFAQSALHPLLLRAPFVYRTFTKPLGYAGDYEMVNQIISDPREGPSTYFQIVNATFLNAAVARAHRNRIEILVQYLSDLATQALAAGRQFKVLNVGCGPAVEIQRFIHQHPEPQQLAFQLVDFSEETLDYTRRQMDNVRHATNKNVDIEFVHESVHQLLKRRVGPDSPEMGEFDAVYCAGLFDYLSDKVCNRLLTHFAARTRKGGTLLVTNVHGSNPEKLSMEHLLEWYLVYRDEARMESLLPAGSANVRLFTDDTGVNVFAQARVGDHV

SEQ ID NO: 34 - protein, 809 aa, Oligotropha carboxidovorans
MNIQTTVEPTSAERAEKLQGMGCKRKRVEDIRFTQGKGNYVDDVKLPGMLFGDFVRSSHAHARIKSIDTSKAKALPGVFAVLTAADLKPLNLHYMPTLAGDVQAVLADEKVLFQNQEVAFVVAKDRYVAADAIELVEVDYEPLPVLVDPFKAMEPDAPLLREDIKDKMTGAHGARKHHNHIFRWEIGDKEGTDATFAKAEVVSKDMFTYHRVHPSPLETCQCVASMDKIKGELTLWGTFQAPHVIRTVVSLISGLPEHKIHVIAPDIGGGFGNKVGAYSGYVCAVVASIVLGVPVKWVEDRMENLSTTSFARDYHMTTELAATKDGKILAMRCHVLADHGAFDACADPSKWPAGFMNICTGSYDMPVAHLAVDGVYTNKASGGVAYRCSFRVTEAVYAIERAIETLAQRLEMDSADLRIKNFIQPEQFPYMAPLGWEYDSGNYPLAMKKAMDTVGYHQLRAEQKAKQEAFKRGETREIMGIGISFFTEIVGAGPSKNCDILGVSMFDSAEIRIHPTGSVIARMGTKSQGQGHETTYAQIIATELGIPADDIMIEEGNTDTAPYGLGTYGSRSTPTAGAATAVAARKIKAKAQMIAAHMLEVHEGDLEWDVDRFRVKGLPEKFKTMKELAWASYNSPPPNLEPGLEAVNYYDPPNMTYPFGAYFCIMDIDVDTGVAKTRRFYALDDCGTRINPMIIEGQVHGGLTEAFAVAMGQEIRYDEQGNVLGASFMDFFLPTAVETPKWETDYTVTPSPHHPIGAKGVGESPHVGGVPCFSNAVNDAYAFLNAGHIQMPHDAWRLWKVGEQLGLHV

SEQ ID NO: 35 - protein, 288 aa, Oligotropha carboxidovorans
MIPGSFDYHRPKSIADAVALLTKLGEDARPLAGGHSLIPIMKTRLATPEHLVDLRDIGDLVGIREEGTDVVIGAMTTQHALIGSDFLAAKLPIIRETSLLIADPQIRYMGTIGGNAANGDPGNDMPALMQCLGAAYELTGPEGARIVAARDYYQGAYFTAIEPGELLTAIRIPVPPTGHGYAYEKLKRKIGDYATAAAAVVLTMSGGKCVTASIGLTNVANTPLWAEEAGKVLVGTALDKPALDKAVALAEAITAPASDGRGPAEYRTKMAGVMLRRAVERAKARAKN

SEQ ID NO: 36 - protein, 166 aa, Oligotropha carboxidovorans
MAKAHIELTINGHPVEALVEPRTLLIHFIREQQNLTGAHIGCDTSHCGACTVDLDGMSVKSCTMFAVQANGASITTIEGMAAPDGTLSALQEGFRMMHGLQCGYCTPGMIMRSHRLLQENPSPTEAEIRFGIGGNLCRCTGYQNIVKAIQYAAAKINGVPFEEAAE